(19) United States
(12) Patent Application Publication
(10) Pub. No.: US 2012/0083476 A1
(43) Pub. Date: Apr. 5, 2012

(54) HETEROARYL-SUBSTITUTED SPIROCYCLIC DIAMINE UREA MODULATORS OF FATTY ACID AMIDE HYDROLASE

(75) Inventors: J. Guy Breitenbucher, Escondido, CA (US); John M. Keith, San Diego, CA (US); William M. Jone, San Diego, CA (US)

(73) Assignee: Janssen Pharmaceutica NV, Beerse (BE)

(21) Appl. No.: 13/375,868

(22) PCT Filed: Jun. 4, 2010
(86) PCT No.: PCT/US10/37402

§ 371 Date: Dec. 2, 2011

Related U.S. Application Data
(60) Provisional application No. 61/184,620, filed on Jun. 5, 2009.

(51) Int. Cl. A61K 31/616; A61K 31/444; A61K 31/438; C07D 487/10; A61K 31/4439; A61K 31/437; C07D 487/04; A61K 31/5025; A61K 31/4427; A61K 31/506; A61K 31/501; A61K 31/5377; C07D 498/04; A61K 31/4709; C12N 9/99; A61P 25/22; A61P 25/24; A61P 25/04; A61P 25/20; A61P 25/00; A61P 3/04; A61P 29/00; A61P 25/14; A61P 43/00; A61P 25/28; A61P 25/08; A61P 25/16; A61P 27/02; A61P 37/06; A61P 25/30; A61P 1/08; A61P 15/00; A61P 27/06; A61P 1/00; A61P 37/00; A61P 1/04; A61P 1/12; A61P 9/12; A61P 35/00; A61P 1/16; A61P 11/00; A61P 17/04; A61P 3/10; A61P 3/00; A61P 19/10; C07D 471/10

(52) U.S. Cl. 514/161; 546/16; 514/278; 514/210.16; 546/15; 544/230; 544/70; 435/184

(57) ABSTRACT

Certain heteroaryl-substituted spirocyclic diamine urea compounds are described, which are useful as FAAH inhibitors. Such compounds may be used in pharmaceutical compositions and methods for the treatment of disease states, disorders, and conditions mediated by fatty acid amide hydrolase (FAAH) activity, such as anxiety, pain, inflammation, sleep disorders, eating disorders, energy metabolism disorders, and movement disorders (e.g., multiple sclerosis).

### CROSS REFERENCE TO RELATED APPLICATION

This application claims the benefit of U.S. provisional patent application Ser. No. 61/184,620, filed Jun. 5, 2009.

### FIELD OF THE INVENTION

The present invention relates to certain heteroaryl-substituted spirocyclic diamine urea compounds, pharmaceutical compositions containing them, and methods of using them for the treatment of disease states, disorders, and conditions mediated by fatty acid amide hydrolase (FAAH) activity.

### BACKGROUND OF THE INVENTION

Medicinal benefits have been attributed to the cannabis plant for centuries. The primary bioactive constituent of cannabis is Δ⁹-tetrahydro-cannabinol (THC). The discovery of THC eventually led to the identification of two endogenous cannabinoid receptors responsible for its pharmacological actions, namely CB₁ and CB₂ (Goya, Exp. Opin. Ther. Patents 2000, 10, 1529). These discoveries not only established the site of action of THC, but also inspired inquiries into the endogenous agonists of these receptors, or “endocannabinoids”. The first endocannabinoid identified was the fatty acid amide anandamide (AEA). AEA itself elicits many of the pharmacological effects of exogenous cannabinoids (Piomelli, Nat. Rev. Neurosci. 2003, 4(11), 873).

The catabolism of AEA is primarily attributable to the integral membrane bound protein fatty acid amide hydrolase (FAAH), which hydrolyzes AEA to arachidonic acid. FAAH was characterized in 1996 by Cravatt and co-workers (Cravatt, Nature 1996, 384, 83). It was subsequently determined that FAAH is additionally responsible for the catabolism of a large number of important lipid signaling fatty acid amides including: another major endocannabinoid, 2-arachidonoylglycerol (2-AG) (Science 1992, 258, 1946-1949); the sleep-inducing substance, oleamide (OEA) (Science 1995, 268, 1506); the appetite-suppressing agent, N-oleoylethanolamine (Rodriguez de Fonesca, Nature 2001, 414, 209); and the anti-inflammatory agent, palmitoylethanolamide (PEA) (Lambert, Curr. Med. Chem. 2002, 9(6), 663).

Small-molecule inhibitors of FAAH should elevate the concentrations of these endogenous signaling lipids and thereby produce their associated beneficial pharmacological effects. There have been some reports of the effects of various FAAH inhibitors in pre-clinical models. In particular, two carbamate-based inhibitors of FAAH were reported to have analgesic properties in animal models. In rats, BMS-1 (see WO 02/087569), which has the structure shown below, was reported to have an analgesic effect in the Chung spinal nerve ligation model of neuropathic pain, and the Hargraves test of acute thermal nociception. URB-597 was reported to have efficacy in the zero plus maze model of anxiety in rats, as well as analgesic efficacy in the rat hot plate and formalin tests (Kathuria, Nat. Med. 2003, 9(1), 76). The sulfonylfluoride AM374 was also shown to significantly reduce spasticity in chronic relapsing experimental autoimmune encephalomyelitis (CREAE) mice, an animal model of multiple sclerosis (Baker, FASEB J. 2001, 15(2), 300).

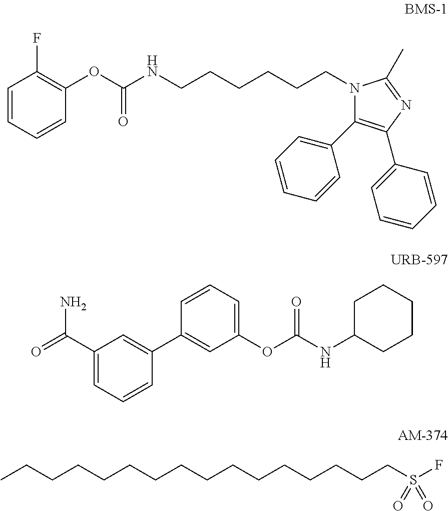

In addition, the oxazolopyridine ketone OL-135 is reported to be a potent inhibitor of FAAH, and has been reported to have analgesic activity in both the hot plate and tail emersion tests of thermal nociception in rats (WO 04/033652).

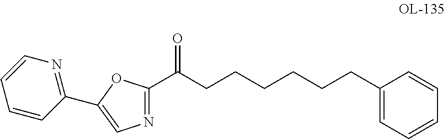

Results of research on the effects of certain exogenous cannabinoids has elucidated that a FAAH inhibitor may be useful for treating various conditions, diseases, disorders, or symptoms. These include pain, nausea/emesis, anorexia, spasticity, movement disorders, epilepsy and glaucoma. To date, approved therapeutic uses for cannabinoids include the relief of chemotherapy-induced nausea and emesis among patients with cancer and appetite enhancement in patients with HIV/AIDS who experience anorexia as a result of wasting syndrome. Two products are commercially available in some countries for these indications, namely, dronabinol (Marinol®) and nabilone.

Apart from the approved indications, a therapeutic field that has received much attention for cannabinoid use is analgesia, i.e., the treatment of pain. Five small randomized controlled trials showed that THC is superior to placebo, producing dose-related analgesia (Robson, Br. J. Psychiatry 2001, 178, 107-115). Atlantic Pharmaceuticals is reported to be developing a synthetic cannabinoid, CT-3, a 1,1-dimethyl heptyl derivative of the carboxylic metabolite of tetrahydrocannabinol, as an orally active analgesic and anti-inflammatory agent. A pilot phase II trial in chronic neuropathic pain with CT-3 was reportedly initiated in Germany in May 2002.

A number of individuals with locomotor activity-related diseases, such as multiple sclerosis have claimed a benefit from cannabis for both disease-related pain and spasticity, with support from small controlled trials (Croxford et el., J. Neuroimmunol, 2008, 193, 120-9; Svendsen, Br. Med. J. 2004, 329, 253). Likewise, various victims of spinal cord injuries, such as paraplegia, have reported that their painful spasms are alleviated after smoking marijuana. A report showing that cannabinoids appear to control spasticity and tremor in the CREAE model of multiple sclerosis demonstrated that these effects are mediated by CB₁ and CB₂ receptors (Baker, Nature 2000, 404, 84-87). Phase 3 clinical trials have been undertaken in multiple sclerosis and spinal cord injury patients with a narrow ratio mixture of tetrahydrocannabinol/cannabidiol (THC/CBD). It has been reported that FAAH knockout mice consistently recover to a better clinical score than wild type controls, and this improvement is not a result of anti-inflammatory activity, but rather may reflect some neuroprotection or remyelination promoting effect of lack of the enzyme (Webb et al, Neurosci Lett., 2008, vol. 439, 106-110).

Reports of small-scale controlled trials to investigate other potential commercial uses of cannabinoids have been made. Trials in volunteers have been reported to have confirmed that oral, injected, and smoked cannabinoids produced dose-related reductions in intraocular pressure (IOP) and therefore may relieve glaucoma symptoms. Ophthalmologists have prescribed cannabis for patients with glaucoma in whom other drugs have failed to adequately control intraocular pressure (Robson, 2001, supra).

Inhibition of FAAH using a small-molecule inhibitor may be advantageous compared to treatment with a direct-acting CB₁ agonist. Administration of exogenous CB₁ agonists may produce a range of responses, including reduced nociception, catalepsy, hypothermia, and increased feeding behavior. These four in particular are termed the “cannabinoid tetrad.” Experiments with FAAH −/− mice show reduced responses in tests of nociception, but did not show catalepsy, hypothermia, or increased feeding behavior (Cravatt, Proc. Natl. Acad. Sci. USA 2001, 98(16), 9371). Fasting caused levels of AEA to increase in rat limbic forebrain, but not in other brain areas, providing evidence that stimulation of AEA biosynthesis may be anatomically regionalized to targeted CNS pathways (Kirkham, Br. J. Pharmacol. 2002, 136, 550). The finding that AEA increases are localized within the brain, rather than systemic, suggests that FAAH inhibition with a small molecule could enhance the actions of AEA and other fatty acid amides in tissue regions where synthesis and release of these signaling molecules is occurring in a given pathophysiological condition (Piomelli, 2003, supra).

In addition to the effects of a FAAH inhibitor on AEA and other endocannabinoids, inhibitors of FAAH's catabolism of other lipid mediators may be used in treating certain other therapeutic indications. For example, PEA has demonstrated biological effects in animal models of inflammation (Holt, et al. Br. J. Pharmacol. 2005, 146, 467-476), immunosuppression, analgesia, and neuroprotection (Ueda, J. Biol. Chem. 2001, 276(38), 35552). Oleamide, another substrate of FAAH, induces sleep (Boger, Proc. Natl. Acad. Sci. USA 2000, 97(10), 5044; Mendelson, Neuropsychopharmacology 2001, 25, S36). Inhibition of FAAH has also been implicated in cognition (Varvel et al., J. Pharmacol. Exp. Ther. 2006, 317(1), 251-257) and depression (Gobbi et al., Proc. Natl. Acad. Sci. USA 2005, 102(51), 18620-18625).

Two additional indications for FAAH are supported by recent data indicating that FAAH substrate activated receptors are important in energy metabolism, and in bone homeostasis (Overton et al., Br. J. Pharmacol. 2008, in press; and Plutzky, Diab. Vasc. Dis. Res. 2007, 4 Suppl 3, S12-4). It has been shown that the previously mentioned lipid signaling fatty acid amides catabolized by FAAH, oleoylethanolamide (OEA), is one of the most active agonists of the recently de-orphanised GPCR 119 (GPR119) (also termed glucose dependent insulinotropic receptor). This receptor is expressed predominantly in the pancreas in humans and activation improves glucose homeostasis via glucose-dependent insulin release in pancreatic beta-cells. GPR119 agonists can suppress glucose excursions when administered during oral glucose tolerance tests, and OEA has also been shown independently to regulate food intake and body weight gain when administered to rodents, indicating a probable benefit in energy metabolism disorders, such as insulin resistance and diabetes. The FAAH substrate palmitoylethanolamide (PEA) is an agonist at the PPARα receptor. Evidence from surrogate markers in human studies with the PPARα agonist fenofibrate is supportive of the concept that PPARα agonism offers the potential for inducing a coordinated PPARα response that may improve dyslipidaemia, repress inflammation and limit atherosclerosis in patients with the metabolic syndrome or type 2 diabetes. The FAAH substrate anandamide (AEA) is an agonist at the PPARγ receptor. Anandamide treatment induces 3T3-L1 differentiation into adipocytes, as well as triglyceride droplet accumulation and expression of adiponectin (Bouaboula et al., E. J. Pharmacol. 2005, 517, 174-181). Low dose cannabinoid therapy has been shown to reduce atherosclerosis in mice, further suggesting a therapeutic benefit of FAAH inhibition in dyslipidemia, liver steatosis, steatohepatitis, obesity, and metabolic syndrome (Steffens et al., Nature, 2005, 434, 782-6).

Osteoporosis is one of the most common degenerative diseases. It is characterized by reduced bone mineral density (BMD) with an increased risk for bone fractures. CB₂-deficient mice have a markedly accelerated age-related trabecular bone loss and cortical expansion. A CB₂-selective agonism enhances endocortical osteoblast number and activity and restrains trabecular osteoclastogenesis and attenuates ovariectomy-induced bone loss (Ofek et al., Proc. Natl. Acad. Sci. U.S.A. 2006, 103, 696-701). There is a substantial genetic contribution to BMD, although the genetic factors involved in the pathogenesis of human osteoporosis are largely unknown. The applicability to human BMD is suggested by genetic studies in which a significant association of single polymorphisms and haplotypes was found encompassing the CNR2 gene on human chromosome 1p36, demonstrating a role for the peripherally expressed CB₂ receptor in the etiology of osteoporosis (Karsak et al., Hum. Mol. Genet, 2005, 14, 3389-96).

Thus, small-molecule FAAH inhibitors should be useful in treating pain of various etiologies, anxiety, multiple sclerosis and other movement disorders, nausea/emesis, eating disorders, epilepsy, glaucoma, inflammation, immunosuppression, neuroprotection, depression, cognition enhancement, and sleep disorders, and potentially with fewer side effects than treatment with an exogenous cannabinoid.

A number of heteroaryl-substituted ureas have been reported in various publications. Certain substituted thiophene ureas are described in U.S. Pat. No. 6,881,741. Certain ureido-pyrazoles are described in U.S. Pat. No. 6,387,900. Certain benzothiazole amide derivatives are described in US Patent Publication US 2003/149036. Certain ureas are reported as prenyltransferase inhibitors in WO 2003/047569. Piperidinyl ureas are described as histamine H₃ receptor antagonists in U.S. Pat. No. 6,100,279. Piperazinyl ureas are disclosed as calcitonin mimetics in U.S. Pat. Nos. 6,124,299 and 6,395,740. Various ureas are reported as small-molecule FAAH modulators in US Patent Publication Nos. US 2006/173184 and US 2007/0004741, in Intl. Patent Appl. Nos. WO 2008/023720, WO 2008/047229, and WO 2008/024139, and by Cravatt et al. (Biochemistry 2007, 46(45), 13019. Ureas are described as modulators of other targets in U.S. Pat. Appl. Publ. US 2007/270433, and in Intl. Pat. Appl. Publ. Nos. WO 2007/096251 and WO 2006/085108. Certain piperidinyl ureas and piperazinyl ureas have been previously described as FAAH modulators in U.S. Pat. Appl. No. 60/931,920, filed May 25, 2007. Certain azetidine derivatives are disclosed in U.S. Pat. Publ. Nos. US 2008/0070892, US 2008/0076750, an US 2008/0076751. Certain spirocyclic compounds are disclosed in U.S. Pat. Publ. No. 2007/0117824. Certain diazaspiroalkane compounds are disclosed in U.S. Pat. Publ. No. US 2007/0249648. Certain spiroazetidinone derivatives are described in Intl. Pat. Appl. Publ. No. WO 2008/033464. Certain diazaspiroalkanes are described in U.S. Pat. Publ. No. US 2007/0249648. However, there remains a desire for potent FAAH modulators with suitable pharmaceutical properties.

### SUMMARY OF THE INVENTION

Certain heteroaryl-substituted spirocyclic diamine urea derivatives are herein described, which have been found to have FAAH-modulating activity. The invention is directed to the general and preferred embodiments defined, respectively, and by the independent and dependent claims appended hereto, which are incorporated by reference herein.

In one general aspect, the invention is directed to compounds of Formula (I):

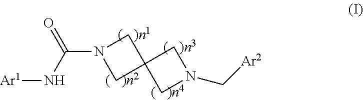

wherein
n¹, n², n³, and n⁴, in the form of sets [n¹,n²,n³,n⁴], are chosen from the following sets, [2,2,1,2], [2,2,1,1], [2,1,0,3], [1,2,1,2], [2,2,2,2], [1,3,2,1], [1,2,2,2], [1,3,2,2], [2,2,1,3], [1,3,3,1], [1,3,1,1], [1,1,2,2], [1,1,1,1], [2,2,0,3], or [1,1,1,3];
Ar¹ is benzo[1,2,5]oxadiazolyl, benzo[d]isoxazolyl, benzooxazol-yl, benzo[d]thiazolyl, imidazo[1,2-a]pyridinyl, imidazo[1,2-b]pyridazinyl, 1H-indazolyl, isoxazolyl, isoxazolo[4,5-b]pyridinyl, isoxazolo[5,4-b]pyridinyl, phenyl, pyrazolyl, 1H-pyrazolo[3,4-b]pyridinyl, pyridazinyl, pyridyl, pyrimidinyl, 1H-pyrrolo[2,3-b]pyridinyl, quinolinyl, or tetrazolyl, with the point of attachment being any substitutable carbon of the respective heterocycle;
   where each Ar¹ is optionally substituted with one or two groups, each said group individually selected from —C₁₋₃alkyl, halo, —CF₃, —CN, —OC₁₋₃alkyl, triazolyl, phenyl, morpholinyl, piperidinyl, or pyrazolyl;
Ar² is
   (i) phenyl optionally substituted with one or two R^{a} moieties;
      where each R^{a} moiety is independently —OH, —CN, halo, —CF₃, —O(CH₂)₀₋₁CF₃, —S(O)(O)C₁₋₄alkyl, —SCF₃, —S(O)(O)CF₃, or two adjacent R^{a} moieties taken together form —OCF₂O—;
   (ii) phenyl substituted at the 3-position with -L-Ar³,
      where L is a linker selected from the group consisting of —O— or —C≡C—; and Ar³ is:
         (a) phenyl optionally substituted with one or two R^{a} moieties; or
         (b) quinolinyl; or
   (iii) napthyl optionally substituted with —OH.

The invention also relates to pharmaceutically acceptable salts of compounds of Formula (I), pharmaceutically acceptable prodrugs of compounds of Formula (I), and pharmaceutically acceptable metabolites of compounds of Formula (I). In certain preferred embodiments, the compound of Formula (I) is a compound selected from those species described or exemplified in the detailed description below.

In a further general aspect, the invention relates to pharmaceutical compositions each comprising: (a) a therapeutically effective amount of at least one chemical entity selected from compounds of Formula (I), pharmaceutically acceptable salts of compounds of Formula (I), pharmaceutically acceptable prodrugs of compounds of Formula (I), and pharmaceutically acceptable metabolites of compounds of Formula (I); and (b) a pharmaceutically acceptable excipient.

In another aspect, embodiments of the invention are useful as FAAH modulators. Thus, the invention is directed to a method for modulating FAAH activity, comprising exposing FAAH to a therapeutically effective amount of at least one chemical entity selected from compounds of Formula (I), pharmaceutically acceptable salts of compounds of Formula (I), pharmaceutically acceptable prodrugs of compounds of Formula (I), and pharmaceutically active metabolites of compounds of Formula (I).

In another general aspect, the invention is directed to a method of treating a subject suffering from or diagnosed with a disease, disorder, or medical condition (collectively, “indications”) mediated by FAAH activity, comprising administering to the subject in need of such treatment a therapeutically effective amount of a compound of Formula (I), a pharmaceutically acceptable salt of a compound of Formula (I), a pharmaceutically acceptable prodrug of a compound of Formula (I), or a pharmaceutically active metabolite of a compound of Formula (I). In preferred embodiments of the inventive method, the disease, disorder, or medical condition is selected from: anxiety, depression, pain, sleep disorders, eating disorders, inflammation, multiple sclerosis and other movement disorders, HIV wasting syndrome, closed head injury, stroke, learning and memory disorders, Alzheimer's disease, epilepsy, Tourette's syndrome, Niemann-Pick disease, Parkinson's disease, Huntington's chorea, optic neuritis, autoimmune uveitis, symptoms of drug or alcohol withdrawal, nausea, emesis, sexual dysfunction, post-traumatic stress disorder, cerebral vasospasm, glaucoma, irritable bowel syndrome, inflammatory bowel disease, immunosuppression, itch, gastroesophageal reflux disease, paralytic ileus, secretory diarrhea, gastric ulcer, rheumatoid arthritis, unwanted pregnancy, hypertension, cancer, hepatitis, allergic airway disease, auto-immune diabetes, intractable pruritis, neuroinflammation, diabetes, metabolic syndrome, and osteoporosis.

Additional embodiments, features, and advantages of the invention will be apparent from the following detailed description and through practice of the invention.

### DETAILED DESCRIPTION OF INVENTION AND ITS PREFERRED EMBODIMENTS

The invention may be more fully appreciated by reference to the following detailed description, including the following glossary of terms and the concluding examples. For the sake of brevity, the disclosures of the publications, including patents and patent applications, cited anywhere in any part of this specification are incorporated herein by reference in their entirety.

As used herein, the terms “including”, “containing” and “comprising” are used in their open, non-limiting sense.

The term “alkyl” refers to a straight- or branched-chain alkyl group having from 1 to 12 carbon atoms in the chain. Such groups may contain saturated or unsaturated carbon atoms within the chain. Examples of alkyl groups include methyl (Me, which also may be structurally depicted by/symbol), ethyl(Et), n-propyl, isopropyl, butyl, isobutyl, sec-butyl, tert-butyl(tBu), pentyl, isopentyl, tert-pentyl, hexyl, isohexyl, prop-2-enyl, prop-2-ynyl, and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

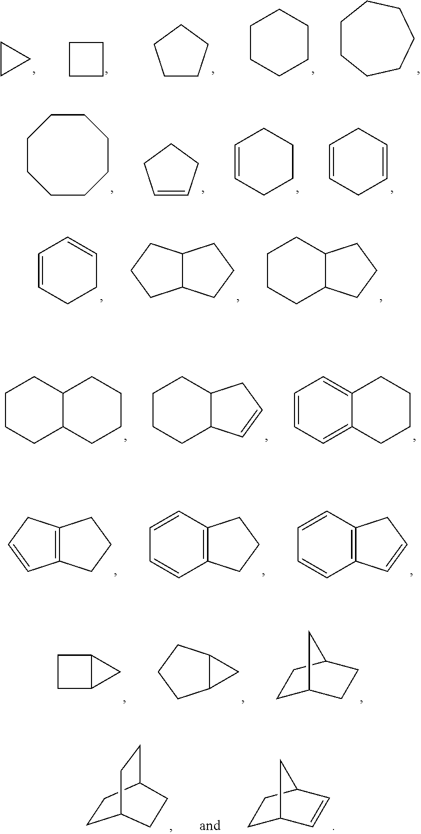

A “heterocycloalkyl” refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. The ring structure may optionally contain up to two oxo groups on carbon or sulfur ring members. Illustrative examples of heterocycloalkyl groups include the following entities, in the form of properly bonded moieties:

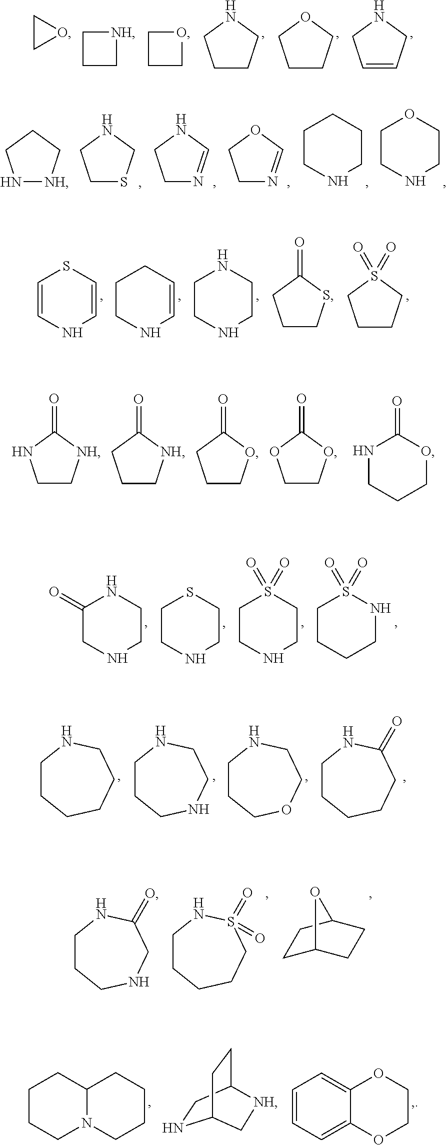

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

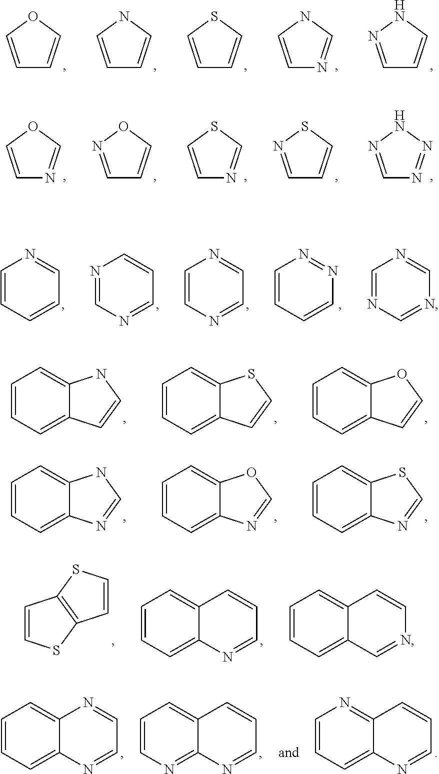

Those skilled in the art will recognize that the species of heteroaryl, cycloalkyl, and heterocycloalkyl groups listed or illustrated above are not exhaustive, and that additional species within the scope of these defined terms may also be selected.

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system, unless indicated otherwise. In cases where a specified moiety or group is not expressly noted as being optionally substituted or substituted with any specified substituent, it is understood that such a moiety or group is intended to be unsubstituted.

A structural formula given herein is intended to represent compounds having structures depicted by the formula as well as equivalent variations or forms. For example, compounds encompassed by Formula (I) may have asymmetric centers and therefore exist in different enantiomeric forms. All optical isomers and stereoisomers of the compounds of the general formula, and mixtures thereof, are considered within the scope of the formula. Thus, a general formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers (e.g. pyrazole, benzimidazole, tetrazole, or benzotriazole tautomers), or as atropisomers, which are intended to be represented by the structural formula. Additionally, a formula given herein is intended to embrace hydrates, solvates, and polymorphs of such compounds, and mixtures thereof, even if such forms are not listed explicitly.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Reference to a chemical entity herein stands for a reference to any one of: (a) the actually recited form of such chemical entity, and (b) any of the forms of such chemical entity in the medium in which the compound is being considered when named. For example, reference herein to a compound such as R—COOH, encompasses reference to any one of, for example, R—COOH(s), R—COOH(sol), and R—COO-(sol). In this example, R—COOH(s) refers to the solid compound, as it could be for example in a tablet or some other solid pharmaceutical composition or preparation; R—COOH(sol) refers to the undissociated form of the compound in a solvent; and R—COO-(sol) refers to the dissociated form of the compound in a solvent, such as the dissociated form of the compound in an aqueous environment, whether such dissociated form derives from R—COOH, from a salt thereof, or from any other entity that yields R—COO— upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In this regard, if such entity is for example in an aqueous environment, it is understood that the compound R—COOH is in such same medium, and therefore the entity is being exposed to species such as R—COOH(aq) and/or R—COO-(aq), where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

Any structural formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸O, ¹⁷O, ³²P, ³³P, ³⁵S, ¹⁸F, ³⁶Cl, and ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT), including drug or substrate tissue distribution assays, or in radioactive treatment of patients. In particular, an ¹⁸F- or ¹¹C-labeled compound may be preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a formula variable appears more than once, the choice of the species from a specified list is independent of the choice of the species for the same variable elsewhere in the formula, unless stated otherwise.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

In some embodiments of Formula (I), Ar¹ is 6-[1,2,3]triazol-2-yl-pyridin-3-yl, benzo[d]isoxazol-3-yl, pyridin-3-yl, 1H-pyrrolo[2,3-b]pyridin-5-yl, 4-chloropyridin-3-yl, 5-fluoro-pyridin-3-yl, quinolin-3-yl, 4-[1,2,3]triazol-2-yl-phenyl, 6-pyrazol-1-yl-pyridin-3-yl, 5-methylpyridin-3-yl, 4-methylpyridin-3-yl, or 2-phenylpyrimidin-5-yl. In further embodiments, Ar¹ is 6-[1,2,3]triazol-2-yl-pyridin-3-yl, benzo[d]isoxazol-3-yl, pyridin-3yl, 1H-pyrrolo[2,3-b]pyridin-5-yl, 4-chloropyridin-3-yl, 5-fluoro-pyridin-3yl, quinolin-3-yl, 4-[1,2,3]triazol-2-yl-phenyl, 6-pyrazol-1-yl-pyridin-3-yl, 5-methylpyridin-3-yl, 4-methylpyridin-3-yl, or 2-phenylpyrimidin-5-yl and n¹, n², n³, and n⁴ are chosen from the following sets [1,1,1,1], [1,1,2,2], [2,2,1,1], [2,2,1,2], or [2,2,2,2].

In some embodiments of Formula (I), Ar¹ is pyridyl optionally substituted with Cl or F. In some embodiments of Formula (I), Ar¹ is optionally substituted with one or two moieties selected from the group consisting of F, Cl, —CH₃, and triazolyl. 2. In some embodiments of Formula (I), Ar² is phenyl substituted at the 3-position with -L-Ar³. In certain embodiments, Ar² is phenyl substituted at the 3-position with -L-Ar³ and L is —O—. In certain embodiments, Ar² is phenyl substituted at the 3-position with -L-Ar³ and Ar³ is phenyl optionally substituted with one or two R^{a} moieties. In certain embodiments, Ar² is phenyl substituted at the 3-position with -L-Ar³ and Ar³ is phenyl optionally substituted with one or two R^{a} moieties, wherein said R^{a} moieties are selected from the group consisting of F, Cl, Br, —CF₃, —OCF₃, —CN, —SO₂CF₃, —SCF₃, and —OCH₂CF₃. In certain embodiments, Ar² is phenyl substituted at the 3-position with -L-Ar³, L is —O— and Ar³ is phenyl optionally substituted with one or two R^{a} moieties. In certain embodiments, Ar² is phenyl substituted at the 3-position with -L-Ar³, L is —O—, Ar³ is phenyl optionally substituted with one or two R^{a} moieties, and Ar¹ is 6-[1,2,3]triazol-2-yl-pyridin-3-yl, benzo[d]isoxazol-3-yl, pyridin-3yl, 1H-pyrrolo[2,3-b]pyridin-5-yl, 4-chloropyridin-3-yl, 5-fluoro-pyridin-3yl, quinolin-3-yl, 4-[1,2,3]triazol-2-yl-phenyl, 6-pyrazol-1-yl-pyridin-3-yl, 5-methylpyridin-3-yl, 4-methylpyridin-3-yl, or 2-phenylpyrimidin-5-yl. In certain embodiments, Ar² is phenyl substituted at the 3-position with -L-Ar³, L is —O—, Ar³ is phenyl optionally substituted with one or two R^{a} moieties, Ar¹ is 6-[1,2,3]triazol-2-yl-pyridin-3-yl, benzo[d]isoxazol-3-yl, pyridin-3yl, 1H-pyrrolo[2,3-b]pyridin-5-yl, 4-chloropyridin-3-yl, 5-fluoro-pyridin-3yl, quinolin-3-yl, 4-[1,2,3]triazol-2-yl-phenyl, 6-pyrazol-1-yl-pyridin-3-yl, 5-methylpyridin-3-yl, 4-methylpyridin-3-yl, or 2-phenylpyrimidin-5-yl and n¹, n², n³, and n⁴ are chosen from the following sets[1,1,1,1], [1,1,2,2], [2,2,1,1], [2,2,1,2], or [2,2,2,2]. In certain embodiments, Ar² is phenyl substituted at the 3-position with -L-Ar³, L is —O—, Ar³ is phenyl optionally substituted with one or two R^{a} moieties and n¹, n², n³, and n⁴ are chosen from the following sets [1,1,1,1], [1,1,2,2], [2,2,1,1], [2,2,1,2], or [2,2,2,2].

In certain embodiments of Formula (I), Ar² is phenyl substituted at the 3-position with -L-Ar³ and L is —C≡O—. In some embodiments, Ar² is phenyl substituted at the 3-position with -L-Ar³, L is —CEO— and Ar¹ is 6-[1,2,3]triazol-2-yl-pyridin-3-yl, benzo[d]isoxazol-3-yl, pyridin-3yl, 1H-pyrrolo[2,3-b]pyridin-5-yl, 4-chloropyridin-3-yl, 5-fluoro-pyridin-3yl, quinolin-3-yl, 4-[1,2,3]triazol-2-yl-phenyl, 6-pyrazol-1-yl-pyridin-3-yl, 5-methylpyridin-3-yl, 4-methylpyridin-3-yl, or 2-phenylpyrimidin-5-yl.

In some embodiments, Ar² is phenyl substituted at the 3-position with -L-Ar³ and Ar³ is quinolinyl. In some embodiments, Ar² is phenyl substituted at the 3-position with -L-Ar³, Ar³ is quinolinyl and n¹, n², n³, and n⁴ are chosen from the following sets [1,1,1,1], [1,1,2,2], [2,2,1,1], [2,2,1,2], or [2,2,2,2]. In some embodiments, Ar² is phenyl substituted at the 3-position with -L-Ar³, Ar³ is quinolinyl, n¹, n², n³, and n⁴ are chosen from the following sets [1,1,1,1], [1,1,2,2], [2,2,1,1], [2,2,1,2], or [2,2,2,2] and Ar¹ is 6-[1,2,3]triazol-2-yl-pyridin-3-yl, benzo[d]isoxazol-3-yl, pyridin-3yl, 1H-pyrrolo[2,3-b]pyridin-5-yl, 4-chloropyridin-3-yl, 5-fluoro-pyridin-3yl, quinolin-3-yl, 4-[1,2,3]triazol-2-yl-phenyl, 6-pyrazol-1-yl-pyridin-3-yl, 5-methylpyridin-3-yl, 4-methylpyridin-3-yl, or 2-phenylpyrimidin-5-yl.

In some embodiments of Formula (I), Ar² is phenyl optionally substituted with one or two R^{a} moieties. In some embodiments, Ar² is phenyl optionally substituted with one or two R^{a} moieties and said R^{a} moieties are independently —OH, —CN, halo, —CF₃, —O(CH₂)₀₋₁CF₃, —S(O)(O)C₁₋₄alkyl, —SCF₃, —S(O)(O)CF₃, or two adjacent R^{a} moieties taken together form —OCF₂O—. In some embodiments, Ar² is phenyl optionally substituted with one or two R^{a} moieties and n¹, n², n³, and n⁴ are chosen from the following sets [1,1,1,1], [1,1,2,2], [2,2,1,1], [2,2,1,2], or [2,2,2,2].

In some embodiments of Formula (I), Ar² is napthyl. In certain embodiments, Ar² is napthyl and Ar¹ is 6-[1,2,3]triazol-2-yl-pyridin-3-yl, benzo[d]isoxazol-3-yl, pyridin-3yl, 1H-pyrrolo[2,3-b]pyridin-5-yl, 4-chloropyridin-3-yl, 5-fluoro-pyridin-3yl, quinolin-3-yl, 4-[1,2,3]triazol-2-yl-phenyl, 6-pyrazol-1-yl-pyridin-3-yl, 5-methylpyridin-3-yl, 4-methylpyridin-3-yl, or 2-phenylpyrimidin-5-yl. In certain embodiments, Ar² is napthyl and Ar¹ is 6-[1,2,3]triazol-2-yl-pyridin-3-yl, benzo[d]isoxazol-3-yl, pyridin-3yl, 1H-pyrrolo[2,3-b]pyridin-5-yl, 4-chloropyridin-3-yl, 5-fluoro-pyridin-3yl, quinolin-3-yl, 4-[1,2,3]triazol-2-yl-phenyl, 6-pyrazol-1-yl-pyridin-3-yl, 5-methylpyridin-3-yl, 4-methylpyridin-3-yl, or 2-phenylpyrimidin-5-yl and n¹, n², n³, and n⁴ are chosen from the following sets [1,1,1,1], [1,1,2,2], [2,2,1,1], [2,2,1,2], or[2,2,2,2]. In certain embodiments, Ar² is napthyl and n¹, n², n³, and n⁴ are chosen from the following sets [1,1,1,1], [1,1,2,2], [2,2,1,1], [2,2,1,2], or [2,2,2,2].

The invention includes also pharmaceutically acceptable salts of the compounds represented by Formula (I), preferably of those described below and of the specific compounds exemplified herein, and methods using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002.

Preferred pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methyl benzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Fleisher et al., Adv. Drug Delivery Rev. 1996, 19, 115-130. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in Robinson et al., J. Med. Chem. 1996, 39, 10-18. Free amines can also be derivatized as amides, sulfonamides or phosphonamides. All of these prodrug moieties may incorporate groups including ether, amine, and carboxylic acid functionalities.

The present invention also relates to pharmaceutically active metabolites of compounds of Formula (I), and uses of such metabolites in the methods of the invention. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

The compounds of Formula (I), and their pharmaceutically acceptable salts, pharmaceutically acceptable prodrugs, and pharmaceutically active metabolites (collectively, “active agents”) of the present invention are useful as FAAH inhibitors in the methods of the invention. The active agents may be used in the inventive methods for the treatment of medical conditions, diseases, or disorders mediated by FAAH, such as those described herein. Active agents according to the invention may therefore be used as an analgesic, anti-depressant, cognition enhancer, neuroprotectant, sedative, appetite stimulant, or contraceptive.

The active agents may be used to treat subjects diagnosed with or suffering from a disease, disorder, or condition mediated through FAAH activity. The term “treat” or “treating” as used herein is intended to refer to administration of an active agent or composition of the invention to a subject for the purpose of effecting a therapeutic or prophylactic benefit through modulation of FAAH activity. Treating includes reversing, ameliorating, alleviating, inhibiting the progress of, lessening the severity of, or preventing a disease, disorder, or condition, or one or more symptoms of such disease, disorder or condition mediated through inhibition of FAAH activity. The term “subject” refers to a mammalian patient in need of such treatment, such as a human. “Modulators” include both inhibitors and activators, where “inhibitors” refer to compounds that decrease, prevent, inactivate, desensitize or down-regulate FAAH expression or activity, and “activators” are compounds that increase, activate, facilitate, sensitize, or up-regulate FAAH expression or activity.

Accordingly, the invention relates to methods of using the active agents described herein to treat subjects diagnosed with or suffering from a disease, disorder, or condition mediated through FAAH activity, such as: anxiety, pain, sleep disorders, eating disorders, inflammation, movement disorders (e.g., multiple sclerosis), energy metabolism (e.g. insulin resistance, diabetes, dyslipidemia, liver steatosis, steatohepatitis, obesity, and metabolic syndrome) and bone homeostasis (e.g. osteoporosis).

In certain preferred embodiments, active agents may be used in methods to treat a FAAH mediated disease, disorder, or medical condition where the disease, disorder, or medical condition is selected from the group consisting of anxiety, depression, pain, sleep disorders, eating disorders, inflammation, multiple sclerosis and other movement disorders, HIV wasting syndrome, closed head injury, stroke, learning and memory disorders, Alzheimer's disease, epilepsy, Tourette's syndrome, epilepsy, Niemann-Pick disease, Parkinson's disease, Huntington's chorea, optic neuritis, autoimmune uveitis, symptoms of drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, autoimmune diabetes, intractable pruritis, neuroinflammation, diabetes, metabolic syndrome, and osteoporosis. In certain preferred embodiments, the disease, disorder, or medical condition is pain or inflammation. In further embodiments, the disease, disorder, or medical condition is anxiety, a sleep disorder, an eating disorder, or a movement disorder. In further embodiments, the disease, disorder, or medical condition is multiple sclerosis. In further embodiments, the disease, disorder, or medical condition is energy metabolism or bone homeostasis.

Symptoms or disease states are intended to be included within the scope of “medical conditions, disorders, or diseases.” For example, pain may be associated with various diseases, disorders, or conditions, and may include various etiologies. Illustrative types of pain treatable with a FAAH-modulating agent, in one example herein a FAAH-inhibiting agent, according to the invention include cancer pain, postoperative pain, GI tract pain, spinal cord injury pain, visceral hyperalgesia, thalamic pain, headache (including stress headache and migraine), low back pain, neck pain, musculoskeletal pain, peripheral neuropathic pain, central neuropathic pain, neurogenerative disorder related pain, and menstrual pain. HIV wasting syndrome includes associated symptoms such as appetite loss and nausea. Parkinson's disease includes, for example, levodopa-induced dyskinesia. Treatment of multiple sclerosis may include treatment of symptoms such as spasticity, neurogenic pain, central pain, or bladder dysfunction. Symptoms of drug withdrawal may be caused by, for example, addiction to opiates or nicotine. Nausea or emesis may be due to chemotherapy, postoperative, or opioid related causes. Treatment of sexual dysfunction may include improving libido or delaying ejaculation. Treatment of cancer may include treatment of glioma. Sleep disorders include, for example, sleep apnea, insomnia, and disorders calling for treatment with an agent having a sedative or narcotic-type effect. Eating disorders include, for example, anorexia or appetite loss associated with a disease such as cancer or HIV infection/AIDS.

In treatment methods according to the invention, an effective amount of at least one active agent according to the invention is administered to a subject suffering from or diagnosed as having such a disease, disorder, or condition. A “therapeutically effective amount” or “effective amount” means an amount or dose of a FAAH-modulating agent sufficient to generally bring about a therapeutic benefit in patients in need of treatment for a disease, disorder, or condition mediated by FAAH activity. Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.0001 to about 200 mg of active agent per kg of subject's body weight per day, preferably about 0.001 to 100 mg/kg/day, or about 0.01 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 5 g/day. Once improvement of the patient's disease, disorder, or condition has occurred, the dose may be adjusted for maintenance treatment. For example, the dosage or the frequency of administration, or both, may be reduced as a function of the symptoms, to a level at which the desired therapeutic effect is maintained. Of course, if symptoms have been alleviated to an appropriate level, treatment may cease. Patients may, however, require intermittent treatment on a long-term basis upon any recurrence of symptoms.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the active agents of the invention may be used in combination with additional active ingredients in the treatment of the above conditions. The additional active ingredients may be coadministered separately with an active agent of Formula (I) or included with such an agent in a pharmaceutical composition according to the invention. In an exemplary embodiment, additional active ingredients are those that are known or discovered to be effective in the treatment of conditions, disorders, or diseases mediated by FAAH activity, such as another FAAH modulator or a compound active against another target associated with the particular condition, disorder, or disease. The combination may serve to increase efficacy (e.g., by including in the combination a compound potentiating the potency or effectiveness of an active agent according to the invention), decrease one or more side effects, or decrease the required dose of the active agent according to the invention. In one illustrative embodiment, a composition according to the invention may contain one or more additional active ingredients selected from opioids, non-steroidal anti-inflammatory drugs (e.g., ibuprofen, cyclooxygenase-2 (COX-2) inhibitors, and naproxen), gabapentin, pregabalin, tramadol, acetaminophen, and aspirin.

The active agents of the invention are used, alone or in combination with one or more additional active ingredients, to formulate pharmaceutical compositions of the invention. A pharmaceutical composition of the invention comprises: (a) an effective amount of at least one active agent in accordance with the invention; and (b) a pharmaceutically acceptable excipient.

When referring to modulating the target receptor, an “effective amount” means an amount sufficient to affect the activity of such receptor. Measuring the activity of the target receptor may be performed by routine analytical methods. Target receptor modulation is useful in a variety of settings, including assays.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the active agents of the invention can be provided in the form of tablets or capsules, or as a solution, emulsion, or suspension. To prepare the oral compositions, the active agents may be formulated to yield a dosage of, e.g., from about 5 mg to 5 g daily, or from about 50 mg to 5 g daily, in single or divided doses. For example, a total daily dosage of about 5 mg to 5 g daily may be accomplished by dosing once, twice, three, or four times per day.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

Active agents may alternatively be administered in methods of this invention by inhalation, via the nasal or oral routes, e.g., in a spray formulation also containing a suitable carrier.

Exemplary active agents useful in methods of the invention will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I).

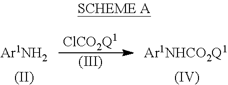

Referring to Scheme A, a carbamate of formula (IV) may be obtained by reacting a compound of formula (II) with a compound of formula (III), in which Q¹ represents an aryl group, under chloroformate condensation conditions. In certain embodiments, Q¹ is phenyl, and the reaction occurs with or without a base, in a solvent such as acetonitrile, at a temperature from about 0° C. to about 80° C. In further embodiments, Q¹ is phenyl and the reaction occurs in pyridine at room temperature (rt). In further embodiments, Q¹ is phenyl and the reaction occurs in acetonitrile at 50° C. without added base.

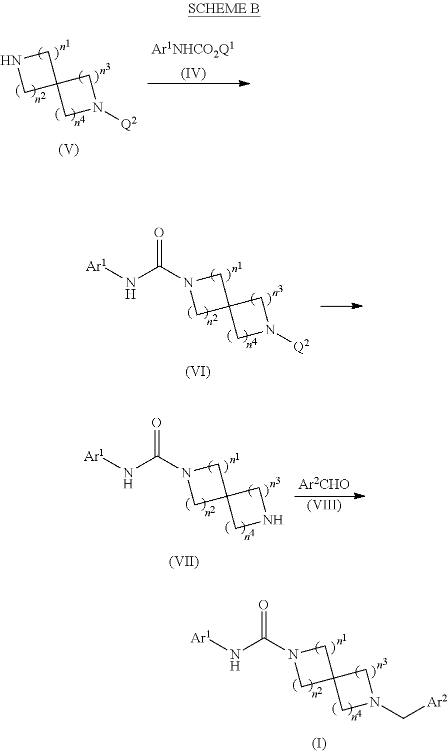

Referring to Scheme B, compounds of formula (I) are prepared from compounds of formula (V). Compounds of formula (V) may be purchased or prepared according to Wuitschik et al., Angew Chem. Int Ed., 2008, 47, 4512 and Burkhard et al., Org. Lett., 2008, 10, 3525. Moiety Q² is a suitable nitrogen protecting group compatible with the transformations described. Preferably, Q² is tert-butyl-carbamoyl (BOC). Compounds of formula (VI) can be prepared by reaction between an intermediate of formula (V) with a carbamate of formula (IV) using appropriate aryl carbamate condensation conditions. In certain embodiments, the reaction may take place in a solvent at a temperature from about rt to about 120° C. In preferred embodiments, Q¹ is phenyl and the reaction is performed in dimethylsulfoxide in a microwave reactor at about 100° C. or by conventional heating from about rt to about 50° C.

Alternatively, a compound of formula (VI) is obtained by reacting a compound of formula (V) with a compound of formula (II) in the presence of di-(N-succin imidyl)carbonate.

A compound of formula (VII) is obtained by Q² removal. Where Q² is BOC, a compound of Formula (VII) is obtained by removing the BOC group by treatment of compound of Formula (VI) with HCl, trifluoroacetic acid (TFA), or formic acid in a solvent such as diethyl ether (Et₂O), DCM, or 1,4-dioxane. Alternatively, BOC removal may be effected in neat TFA or formic acid. A compound of formula (I) is formed by reacting a compound of formula (VII) with an aldehyde of formula (VIII) under reductive amination conditions in the presence of a reductant such as sodium triacetoxyborohydride, resin-supported triacetoxyborohyd ride (e.g., MP-B(OAc)₃H), sodium cyanoborohydride, or phenylsilane in a solvent such as tetrahydrofuran (THF), 1,2-dichloroethane (DCE), DCM, methanol (MeOH), ethanol (EtOH), or Et₂O at a temperature from about 0° C. to 80° C. The use of a promoter or catalyst with acidic character such as an organometallic complex or carboxylic acid may increase the rate of the reaction and/or reduce the formation of by-products. In preferred embodiments, sodium triacetoxyborohydride in DCE is employed at rt.

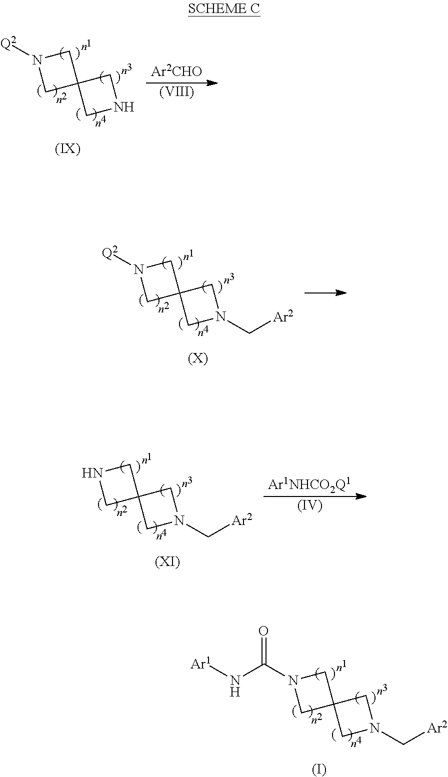

Referring to Scheme C, compounds of Formula (I) may be alternatively prepared from compounds of formula (IX). Compounds of formula (IX) may be purchased or prepared according to procedures in the literature (see Wuitschik et al., Angew Chem. Int Ed., 2008, 47, 4512; Burkhard et al., Org. Lett., 2008, 10, 3525). A compound of formula (X) is obtained by reacting an aldehyde (VIII) with a compound of formula (IX) under reductive amination conditions as described previously in Scheme B. Deprotection of Q² from a compound of formula (X) under general deprotection conditions provides compounds of formula (XI). In preferred embodiments, Q² is BOC. A compound of Formula (I) is obtained by reacting a compound of formula (XI) with either a compound of formula (IV) or with a compound Ar¹ NH₂ in the presence of di-(N-succinimidyl) carbonate.

Compounds of Formula (I) may be converted to their corresponding salts by applying general techniques described in the art. For example, a compound of Formula (I) may be treated with trifluoroacetic acid, HCl, or citric acid in a solvent such as Et₂O, 1,4-dioxane, DCM, THF, or MeOH to provide the corresponding salt forms.

Compounds prepared according to the schemes described above may be obtained as single enantiomers or diastereomers by enantio- or diastero-specific synthesis, or by resolution. Compounds prepared according to the schemes above may alternatively be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following specific examples are provided to further illustrate the invention and various preferred embodiments.

### EXAMPLES

Chemistry:

In preparing the examples listed below, the following general experimental and analytical methods were used.

Reaction mixtures were stirred under a nitrogen atmosphere at room temperature (rt) unless otherwise noted. Where solutions or mixtures are concentrated, they are typically concentrated under reduced pressure using a rotary evaporator. Where solutions are dried, they are typically dried over a drying agent such as MgSO₄ or Na₂SO₄, unless otherwise noted.

Microwave reactions were carried out in either a CEM Discover or a Biotage Initiator™ Microwave at specified temperatures.

Normal phase flash column chromatography (FCC) was performed on silica gel columns using ethyl acetate (EtOAc)/hexanes as eluent, unless otherwise indicated.

Reversed-Phase High Performance Liquid Chromatography (HPLC) was performed using: Shimadzu instrument with a Phenomenex Gemini column 5 μm C18 (150×21.2 mm) or Waters Xterra RP18 OBD column 5 μm (100×30 mm), a gradient of 95:5 to 0:100 water (0.05% TFA)/CH₃CN (0.05% TFA), a flow rate of 80 mL/min, and detection at 254 nM.

Mass spectra were obtained on an Agilent series 1100 MSD using electrospray ionization (ESI) in positive mode unless otherwise indicated.

NMR spectra were obtained on either a Bruker model DPX400 (400 MHz), DPX500 (500 MHz) or DRX600 (600 MHz) spectrometer. The format of the ¹H NMR data below is: chemical shift in ppm down field of the tetramethylsilane reference (multiplicity, coupling constant J in Hz, integration).

Chemical names were generated using ChemDraw Ultra 6.0.2 (CambridgeSoft Corp., Cambridge, Mass.) or ACD/Name Version 9 (Advanced Chemistry Development, Toronto, Ontario, Canada).

### Intermediate 1: 2,6-Diaza-spiro[3.3]heptane-2-carboxylic acid tert-butyl ester, oxalic acid salt

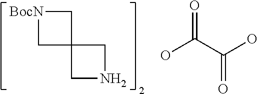

6-(Toluene-4-sulfonyl)-2-oxa-6-aza-spiro[3.3]heptane. To a solution of tribromopentaerythitol (17.886 g, 55 mmol) and p-toluenesulfonamide (11.301 g, 66 mmol) in EtOH (200 mL) was added KOH (9.875 g, 176 mmol). The reaction vessel was purged with N₂ and heated to reflux (90° C.) for three days. The solvent was evaporated in vacuo and the product precipitated by stirring in 1 M KOH (100 mL) for 2 h. The crude solid was purified (FCC) to give 6-(toluene-4-sulfonyl)-2-oxa-6-aza-spiro[3.3]heptane as a white solid (8.939 g, 64%). MS (ESI⁺): calcd for C₁₂H₁₅NO₃S m/z 253.08; found 254.1 (M+H)⁺. ¹H NMR (CDCl₃): 7.71 (d, J=8.2, 2H), 7.37 (d, J=8.4, 2H), 4.58 (s, 4H), 3.91 (s, 4H).

[3-Bromomethyl-1-(toluene-4-sulfonyl)-azetidin-3-yl]-methanol. To a cooled suspension (0° C.) of 6-(toluene-4-sulfonyl)-2-oxa-6-aza-spiro[3.3]heptane (3.246 g, 12.81 mmol) in Et₂O (130 mL) was added 30% HBr/HOAc (4.490 mL, 16.65 mmol) in Et₂O (50 mL) dropwise over 15 min. After 10 min of stirring the reaction was quenched by the slow addition of saturated aq. NaHCO₃ (300 mL). The organic layer was isolated and then washed with saturated aq. NaCl (2×100 mL). The organic layer was dried over Na₂SO₄, filtered and concentrated to dryness, yielding a solid product. The crude product was purified (FCC) to give [3-bromomethyl-1-(toluene-4-sulfonyl)-azetidin-3-yl]-methanol as a white crystalline solid (3.892 g, 91%). MS (ESI⁺): calcd for C₁₂H₁₆BrNO₃S m/z 333.00; found 334.0 (M+H)⁺. ¹H NMR (CDCl₃): 7.73 (d, J=8.3, 2H), 7.38 (d, J=7.9, 2H), 3.68 (s, 2H), 3.62 (d, J=8.5, 2H), 3.55 (d, J=8.5, 2H), 3.45 (s, 2H), 2.47 (s, 3H).

3,3-Bis-bromomethyl-1-(toluene-4-sulfonyl)-azetidine. To a cooled solution (0° C.) of [3-bromomethyl-1-(toluene-4-sulfonyl)-azetidin-3-yl]-methanol (3.842 g, 11.5 mmol) and CBr₄ (6.371 g, 19.21 mmol) in CH₂Cl₂ was added Ph₃P (5.012 g, 19.21 mmol). The reaction mixture was stirred overnight with gradual warming to rt. The solvent was evaporated in vacuo and the crude solid purified (FCC) to give 3,3-bis-bromomethyl-1-(toluene-4-sulfonyl)-azetidine as a white crystalline solid (3.625 g, 79%). ¹H NMR (CDCl₃): 7.73 (d, J=8.3, 2H), 7.40 (d, J=7.9, 2H), 3.60 (s, 4H), 3.53 (s, 4H), 2.47 (s, 3H).

2-Benzyl-6-(toluene-4-sulfonyl)-2,6-diaza-spiro[3.3]heptane. To a solution of 3,3-bis-bromomethyl-1-(toluene-4-sulfonyl)-azetidine (6.983 g, 17.58 mmol) in MeCN (125 mL) was added benzyl amine (3.840 mL, 35.16 mmol) and DIPEA (15.311 mL, 87.9 mmol). The reaction mixture was heated at reflux (95° C.) for 2 d. The solvent was evaporated in vacuo and the residue diluted with CH₂Cl₂ (150 mL) and washed with 1M NaOH (100 mL). The organic layer was separated, dried over Na₂SO₄, filtered and concentrated to dryness. The crude product was purified (FCC) to give 2-benzyl-6-(toluene-4-sulfonyl)-2,6-diaza-spiro[3.3]heptane as a white solid (5.089 g, 85%). ¹H NMR 7.70 (d, J=8.3, 2H), 7.34 (d, J=8.0, 2H), 7.30-7.21 (m, 3H), 7.19-7.15 (m, 2H), 3.82 (s, 4H), 3.47 (s, 2H), 3.12 (s, 4H), 2.43 (s, 3H).

6-(Toluene-4-sulfonyl)-2,6-diaza-spiro[3.3]heptane-2-carboxylic acid tert-butyl ester. To a solution of 2-benzyl-6-(toluene-4-sulfonyl)-2,6-diaza-spiro[3.3]heptane (5.089 g, 14.86 mmol) in MeOH (75 mL) was added 10% Pd/C (1.0 g). The reaction was stirred at 50° C. for 3 d under a H₂ atmosphere. The Pd was removed by filtering through celite. BOC₂O (3.405 g, 15.6 mmol) was added and the reaction stirred for 1 h at rt. The solvent was removed in vacuo and the crude residue purified (FCC) to give 6-(toluene-4-sulfonyl)-2,6-diaza-spiro[3.3]heptane-2-carboxylic acid tert-butyl ester as a white solid (3.426 g, 65%). ¹H NMR 7.71 (d, J=8.3, 2H), 7.37 (d, J=7.9, 2H), 3.85 (d, J=4.0, 8H), 2.46 (s, 3H), 1.39 (s, 9H).

2,6-Diaza-spiro[3.3]heptane-2-carboxylic acid tert-butyl ester, oxalic acid. To a solution of 6-(toluene-4-sulfonyl)-2,6-diaza-spiro[3.3]heptane-2-carboxylic acid tert-butyl ester (0.852 g, 2.42 mmol) and MeOH (25 mL) was added powdered Mg (0.471 g, 19.36 mmol). The suspension was sonicated at rt for 45 min. The solvent was removed in vacuo and the residue suspended in Et₂O (50 mL). Na₂SO₄.10H₂O (10 g) was added and the mixture stirred for 1 h at which point it was filtered, dried over Na₂SO₄ and filtered again. A solution of anhydrous oxalic acid (0.109 g, 1.21 mmol) in EtOH (1 mL) was added to precipitate the final product, 2,6-diaza-spiro[3.3]heptane-2-carboxylic acid tert-butyl ester, oxalic acid salt, as a white solid (0.350 g, 59%). MS (ESI⁺): calcd for C₁₀HN₂O₂ m/z 198.14; found 199.2 (M+H)⁺. ¹H NMR (d₄-methanol): 4.83 (s, 1H), 4.22 (s, 4H), 4.10 (s, 4H), 1.42 (s, 9H).

### Intermediate 2: (6-[1,2,3]-Triazol-2-yl-pyridin-3-yl)-carbamic acid phenyl ester

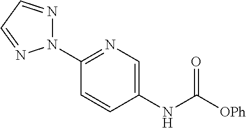

To a solution of 6-[1,2,3]Triazol-2-yl-pyridin-3-ylamine (1.0 g, 6.205) in MeCN (10 mL) was added phenyl chloroformate (0.389 mL, 3.103) dropwise at rt. After 16 h, the reaction mixture was diluted with EtOAc (30 mL) and washed with saturated aq. NaCl. The organic layer was isolated, dried (Na₂SO₄), and concentrated. The crude residue was purified (FCC) to give (6-[1,2,3]triazol-2-yl-pyridin-3-yl)-carbamic acid phenyl ester as a white solid (0.808 g, 93%). MS (ESI⁺): calcd for C₁₄H₁₁N₅O₂ m/z 281.09; found 282.1 (M+H)⁺.

Intermediates 3 to 52 were prepared using methods analogous to those described for Intermediate 2, using the appropriate starting material.

### Intermediate 3: Benzo[d]isoxazol-3-yl-carbamic acid phenyl ester

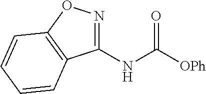

MS (ESI⁺): calcd for C₁₄H₁₀N₂O₃ m/z 254.07; found 255.1 (M+H)⁺.

### Intermediate 4: Pyridin-3-yl-carbamic acid phenyl ester

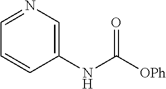

MS (ESI⁺): calcd for C₁₂H₁₀N₂O₂ m/z 214.07; found 215.1 (M+H)⁺.

### Intermediate 5: (1H-Pyrrolo[2,3-b]pyridin-5-yl)-carbamic acid phenyl ester

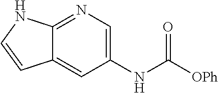

MS (ESI⁺): calcd for C₁₄H₁₁N₃O₂ m/z 253.09; found 254.1 (M+H)⁺.

### Intermediate 6: Imidazo[1,2-b]pyridazin-3-yl-carbamic acid phenyl ester

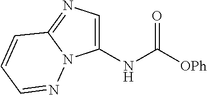

MS (ESI⁺): calcd for C₁₃H₁₀N₄O₂ m/z 254.08; found 255.1 (M+H)⁺.

### Intermediate 7: Imidazo[1,2-a]pyridin-3-yl-carbamic acid phenyl ester

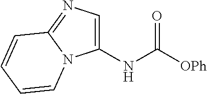

MS (ESI⁺): calcd for C₁₄H₁₁N₃O₂ m/z 253.09; found 254.1 (M+H)⁺.

### Intermediate 8: (4-[1,2,3]-Triazol-2-yl-phenyl)-carbamic acid phenyl ester

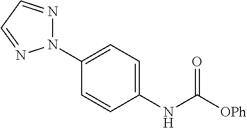

MS (ESI⁺): calcd for C₁₅H₁₂N₄O₂ m/z 280.10; found 281.1 (M+H)⁺.

### Intermediate 9: Pyrimidin-2-yl-carbamic acid phenyl ester

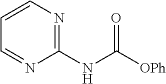

MS (ESI⁺): calcd for C₁₁H₉N₃O₂ m/z 215.07; found 216.1 (M+H)⁺.

### Intermediate 10: Pyrimidin-4-yl-carbamic acid phenyl ester

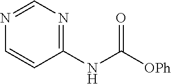

MS (ESI⁺): calcd for C₁₁H₉N₃O₂ m/z 215.07; found 216.1 (M+H)⁺.

### Intermediate 11: Pyridazin-3-yl-carbamic acid phenyl ester

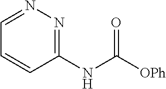

MS (ESI⁺): calcd for C₁₁H₉N₃O₂ m/z 215.07; found 216.1 (M+H)⁺.

### Intermediate 12: (6-Pyrazol-1-yl-pyridin-3-yl)-carbamic acid phenyl ester

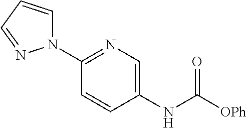

MS (ESI⁺): calcd for C₁₅H₁₂N₄O₂ m/z 280.10; found 281.1 (M+H)⁺.

### Intermediate 13: (6-[1, 2,4]-Triazol-1-yl-pyridin-3-yl)-carbamic acid phenyl ester

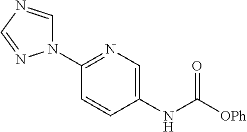

MS (ESI⁺): calcd for C₁₄H₁₁N₅O₂ m/z 281.10; found 282.1 (M+H)⁺.

### Intermediate 14: (6-[1,2,4]-Triazol-4-yl-pyridin-3-yl)-carbamic acid phenyl ester

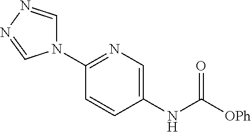

MS (ESI⁺): calcd for C₁₄H₁₁N₅O₂ m/z 281.10; found 282.1 (M+H)⁺.

### Intermediate 15: (6-Chloro-pyridin-3-yl)-carbamic acid phenyl ester

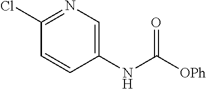

MS (ESI⁺): calcd for C₁₂H₉ClN₂O₂ m/z 248.04; found 249.1 (M+H)⁺.

### Intermediate 16: (6-Methoxy-pyridin-3-yl)-carbamic acid phenyl ester

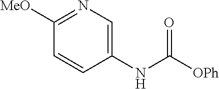

MS (ESI⁺): calcd for C₁₃H₁₂N₂O₃ m/z 244.08; found 249.1 (M+H)⁺.

### Intermediate 17: (6-Cyano-pyridin-3-yl)-carbamic acid phenyl ester

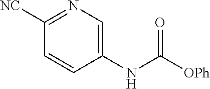

MS (ESI⁺): calcd for C₁₃H₉N₃O₂ m/z 239.07; found 240.1 (M+H)⁺.

### Intermediate 18: (1H-Tetrazol-5-yl)-carbamic acid phenyl ester

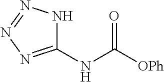

MS (ESI⁺): calcd for C₈H₇N₅O₂ m/z 205.06; found 206.1 (M+H)⁺.

### Intermediate 19: Benzo[1,2,5]oxadiazol-4-yl-carbamic acid phenyl ester

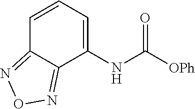

MS (ESI⁺): calcd for C₁₃H₉N₃O₃ m/z 255.06; found 256.1 (M+H)⁺.

### Intermediate 20: (4-Chloro-pyridin-3-yl)-carbamic acid phenyl ester

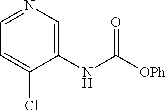

MS (ESI⁺): calcd for C₁₂H₉ClN₂O₂ m/z 248.04; found 249.1 (M+H)⁺.

### Intermediate 21: (2-Chloro-pyridin-3-yl)-carbamic acid phenyl ester.

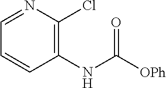

MS (ESI⁺): calcd for C₁₂H₉ClN₂O₂ m/z 248.04; found 249.1 (M+H)⁺.

### Intermediate 22: (6-Morpholin-4-yl-pyridin-3-yl)-carbamic acid phenyl ester

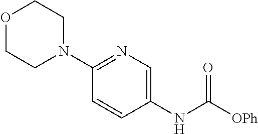

MS (ESI⁺): calcd for C₁₆H₁₇N₃O₃ m/z 299.13; found 300.1 (M+H)⁺.

### Intermediate 23: (1H-Pyrazol-3-yl)-carbamic acid phenyl ester

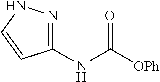

MS (ESI⁺): calcd for C₁₀H₉N₃O₂ m/z 203.07; found 204.1 (M+H)⁺.

### Intermediate 24: (5-Chloro-pyridin-3-yl)-carbamic acid phenyl ester

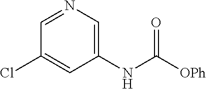

MS (ESI⁺): calcd for C₁₂H₉ClN₂O₂ m/z 248.04; found 249.1 (M+H)⁺.

### Intermediate 25: (6-Fluoro-pyridin-3-yl)-carbamic acid phenyl ester

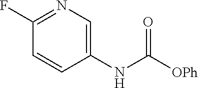

MS (ESI⁺): calcd for C₁₂H₉FN₂O₂ m/z 232.06; found 233.1 (M+H)⁺.

### Intermediate 26: (6-Methoxy-pyrimidin-4-yl)-carbamic acid phenyl ester

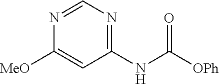

MS (ESI⁺): calcd for C₁₂H₁₁N₃O₃ m/z 245.08; found 246.1 (M+H)⁺.

### Intermediate 27: (6-Chloro-pyridazin-3-yl)-carbamic acid phenyl ester

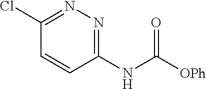

MS (ESI⁺): calcd for C₁₁H₈ClN₃O₂ m/z 249.03; found 250.1 (M+H)⁺.

### Intermediate 28: (1,5-Dimethyl-1H-pyrazol-3-yl)-carbamic acid phenyl ester

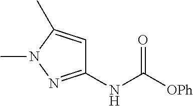

MS (ESI⁺): calcd for C₁₂H₁₃N₃O₂ m/z 231.10; found 232.1 (M+H)⁺.

### Intermediate 29: (4-Bromo-1-methyl-1H-pyrazol-3-yl)-carbamic acid phenyl ester

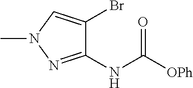

MS (ESI⁺): calcd for C₁₁H₁₀BrN₃O₂ m/z 295.00; found 296.1 (M+H)⁺.

### Intermediate 30: (2-Ethyl-2H-pyrazol-3-yl)-carbamic acid phenyl ester

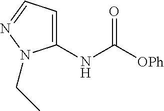

MS (ESI⁺): calcd for C₁₂H₁₃N₃O₂ m/z 231.10; found 232.1 (M+H)⁺.

### Intermediate 31: (2-Methyl-benzooxazol-5-yl)-carbamic acid phenyl ester

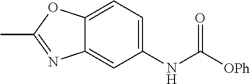

MS (ESI⁺): calcd for C₁₅H₁₂N₂O₃ m/z 268.08; found 269.1 (M+H)⁺.

### Intermediate 32: Isoxazolo[5,4-b]pyridin-3-yl-carbamic acid phenyl ester

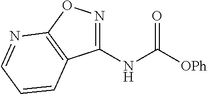

MS (ESI⁺): calcd for C₁₃H₉N₃O₃ m/z 255.06; found 256.1 (M+H)⁺.

### Intermediate 33: Isoxazolo[4,5-b]pyridin-3-yl-carbamic acid phenyl ester

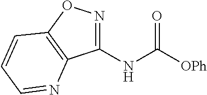

MS (ESI⁺): calcd for C₁₃H₉N₃O₃ m/z 255.06; found 256.1 (M+H)⁺.

### Intermediate 34: (1H-Indazol-7-yl)-carbamic acid phenyl ester

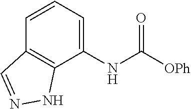

MS (ESI⁺): calcd for C₁₄H₁₁N₃O₂ m/z 253.09; found 254.1 (M+H)⁺.

### Intermediate 35: Imidazo[1,2-a]pyridin-6-yl-carbamic acid phenyl ester

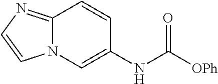

MS (ESI⁺): calcd for C₁₄H₁₁N₃O₂ m/z 253.09; found 254.1 (M+H)⁺.

### Intermediate 36: (6-Methoxy-pyridazin-3-yl)-carbamic acid phenyl ester

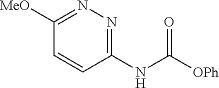

MS (ESI⁺): calcd for C₁₂H₁₁N₃O₃ m/z 245.08; found 246.1 (M+H)⁺.

### Intermediate 37: (2-Trifluoromethyl-pyrimidin-4-yl)-carbamic acid phenyl ester

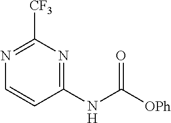

MS (ESI⁺): calcd for C₁₂H₈F₃N₃O₂ m/z 283.06; found 284.1 (M+H)⁺.

### Intermediate 38: (2-Methoxy-pyrimidin-4-yl)-carbamic acid phenyl ester.

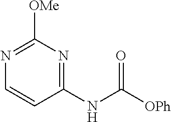

MS (ESI⁺): calcd for C₁₂H₁₁N₃O₃ m/z 245.08; found 246.1 (M+H)⁺.

### Intermediate 39: (5-Fluoro-pyridin-3-yl)-carbamic acid phenyl ester

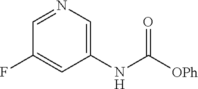

MS (ESI⁺): calcd for C₁₂H₉FN₂O₂ m/z 232.06; found 233.1 (M+H)⁺.

### Intermediate 40: (1H-Pyrrolo[2,3-b]pyridin-4-yl)-carbamic acid phenyl ester

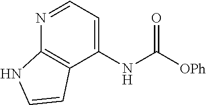

MS (ESI⁺): calcd for C₁₄H₁₁N₃O₂ m/z 253.09; found 254.1 (M+H)⁺.

### Intermediate 41: (1,3-Dimethyl-1H-pyrazolo[3,4-b]pyridin-5-yl)-carbamic acid phenyl ester

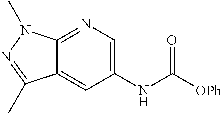

MS (ESI⁺): calcd for C₁₅H₁₄N₄O₂ m/z 282.11; found 283.1 (M+H)⁺.

### Intermediate 42: (5-Methyl-isoxazol-3-yl)-carbamic acid phenyl ester

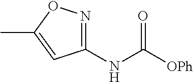

MS (ESI⁺): calcd for C₁₁H₁₀N₂O₃ m/z 218.07; found 219.1 (M+H)⁺.

### Intermediate 43: (2-Methyl-benzothiazol-6-yl)-carbamic acid phenyl ester

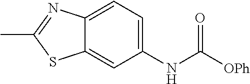

MS (ESI⁺): calcd for C₁₅H₁₂N₂O₂S m/z 284.06; found 285.1 (M+H)⁺.

### Intermediate 44: (5-Methyl-1H-pyrazol-3-yl)-carbamic acid phenyl ester

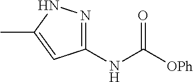

MS (ESI⁺): calcd for Cᵢᵢ Hᵢᵢ N₃O₂ m/z 217.09; found 218.1 (M+H)⁺.

### Intermediate 45: (5-Methyl-pyridin-3-yl)-carbamic acid phenyl ester

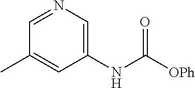

MS (ESI⁺): calcd for C₁₃H₁₂N₂O₂ m/z 228.09; found 229.1 (M+H)⁺.

### Intermediate 46: (2-Fluoro-pyridin-3-yl)-carbamic acid phenyl ester

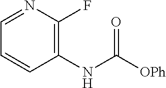

MS (ESI⁺): calcd for C₁₂H₉FN₂O₂ m/z 232.06; found 233.1 (M+H)⁺.

### Intermediate 47: (3,4,5,6-Tetrahydro-2H-[1,2′]bipyridinyl-5′-yl)-carbamic acid phenyl ester

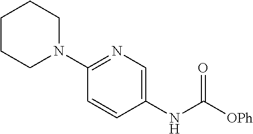

MS (ESI⁺): calcd for C₁₇H₁₉N₃O₂ m/z 297.15; found 298.2 (M+H)⁺.

### Intermediate 48: (5-Bromo-pyridin-3-yl)-carbamic acid phenyl ester

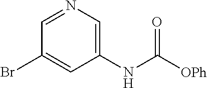

MS (ESI⁺): calcd for C₁₂H₉BrN₂O₂ m/z 291.98; found 293.0 (M+H)⁺.

### Intermediate 49: (2-Phenyl-pyrimidin-5-yl)-carbamic acid phenyl ester

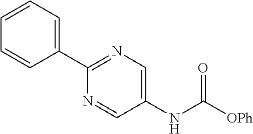

MS (ESI⁺): calcd for C₁₇H₁₃N₃O₂ m/z 291.10; found 292.1 (M+H)⁺.

### Intermediate 50: (4-Cyano-pyridin-3-yl)-carbamic acid phenyl ester

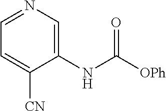

MS (ESI⁺): calcd for C₁₃H₉N₃O₂ m/z 293.07; found 294.1 (M+H)⁺.

### Intermediate 51: (4-Methyl-pyridin-3-yl)-carbamic acid phenyl ester

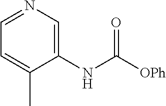

MS (ESI⁺): calcd for C₁₃H₁₂N₂O₂ m/z 228.09; found 229.1 (M+H)⁺.

### Intermediate 52: (4-Trifluoromethyl-pyridin-3-yl)-carbamic acid phenyl ester

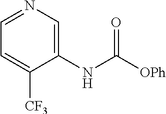

MS (ESI⁺): calcd for C₁₃H₉F₃N₂O₂ m/z 282.06; found 283.1 (M+H)⁺.

### Example 1

### 2-[3-(4-Chloro-phenoxy)-benzyl]-2,8-diaza-spiro[4.5]decane-8-carboxylic acid (6-[1,2,3]triazol-2-yl-pyridin-3-yl)-amide

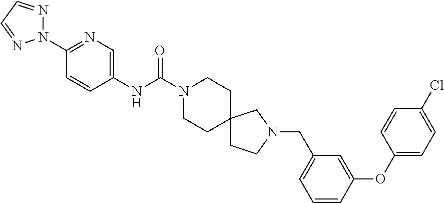

2-[3-(4-Chloro-phenoxy)-benzyl]-2,8-diaza-spiro[4.5]decane-8-carboxylic acid tert-butyl ester. To a suspension of 2,8-diaza-spiro[4.5]decane-8-carboxylic acid tert-butyl ester, hydrochloric acid salt (0.5 g, 1.81 mmol) in THF (10 mL) were added TEA (0.246 mL, 1.81 mmol) and 3-(4-chloro-phenoxy)-benzaldehyde (0.381 mL, 1.99 mmol). After 15 min of stirring, the reaction mixture was treated with NaB(OAc)₃H (0.957 g, 4.52 mmol) and stirred overnight. The reaction was quenched with saturated aq. NaHCO₃ (30 mL). The aqueous phase was extracted with EtOAc (2×30 mL). The organic layers were combined and washed with saturated aq. NaCl (2×50 mL). The organic layer was isolated, dried over Na₂SO₄, filtered and concentrated to dryness. The crude residue was purified (FCC) to give 2-[3-(4-chloro-phenoxy)-benzyl]-2,8-diaza-spiro[4.5]decane-8-carboxylic acid tert-butyl ester as a pale yellow oil (0.584 g, 71%). MS (ESI⁺): calcd for C₂₇H₃₅ClN₂O₂S m/z 456.22; found 457.2 (M+H)⁺.

2-[3-(4-Chloro-phenoxy)-benzyl]-2,8-diaza-spiro[4.5]decane hydrochloride. A solution of 2-[3-(4-chloro-benzyl)-benzyl]-2,8-diaza-spiro[4.5]decane-8-carboxylic acid tert-butyl ester (0.584 g, 1.28 mmol) in CH₂Cl₂ (13 mL) was treated with 4 M HCl/dioxane (1.58 mL) and stirred overnight. The resulting white precipitate was filtered and dried under vacuum to give 2-[3-(4-chloro-phenoxy)-benzyl]-2,8-diaza-spiro[4.5]decane hydrochloride as a white solid (0.433 g, 86%). MS (ESI⁺): calcd for C₂₁H₂₅ClN₂O m/z 356.17; found 357.2 (M+H)⁺.

2-[3-(4-Chloro-phenoxy)-benzyl]-2,8-diaza-spiro[4.5]decane-8-carboxylic acid (6-[1,2,3]triazol-2-yl-pyridin-3-yl)-amide. To a solution of 2-[3-(4-chloro-phenoxy)-benzyl]-2,8-diaza-spiro[4.5]decane hydrochloride (0.050 g, 0.13 mmol) and TEA (0.052 mL, 0.38 mmol) in DMSO (2 mL) was added (6-[1,2,3]Triazol-2-yl-pyridin-3-yl)-carbamic acid phenyl ester (0.039 g, 0.140 mmol). The reaction mixture was heated at 50° C. overnight, then diluted with EtOAc (40 mL) and washed with saturated aq. NaHCO₃ (40 mL). The organic layer was dried (Na₂SO₄) and concentrated. The crude residue was purified (FCC) to give 2-[3-(4-chloro-phenoxy)-benzyl]-2,8-diaza-spiro[4.5]decane-8-carboxylic acid (6-[1,2,3]triazol-2-yl-pyridin-3-yl)-amide (0.049 g, 71%). MS (ESI⁺): calcd for C₂₉H₃₀ClN₇O₂ m/z 543.21; found 544.2 (M+H)⁺. ¹H NMR (CDCl₃): 8.41 (s, 1H), 8.28 (dd, J=9.0, 2.2, 1H), 7.96 (d, J=9.0, 1H), 7.82 (s, 2H), 7.46 (s, 1H), 7.30-7.26 (m, 3H), 7.07 (d, J=7.6, 1H), 6.99 (s, 1H), 6.93 (d, J=8.9, 2H), 6.88 (dd, J=8.1, 1.9, 1H), 3.63 (s, 2H), 3.55-3.40 (m, 4H), 2.67 (s, 2H), 2.46 (s, 2H), 1.69 (t, J=6.8, 2H), 1.62-1.57 (m, 4H).

Examples 2 to 89 were prepared using methods analogous to those described for Example 1, using the appropriate carbamate, BOC-diazaspirocycle and aldehyde.

### Example 2

### 2-[3-(4-Chloro-phenoxy)-benzyl]-2,8-diaza-spiro[4.5]decane-8 carboxylic acid benzo[d]isoxazol-3-ylamide

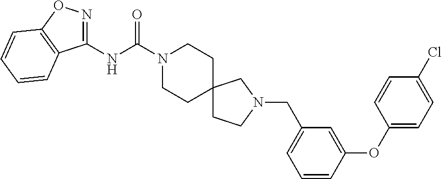

MS (ESI⁺): calcd for C₂₉H₂₉ClN₄O₃ m/z 516.19; found 517.2 (M+H)⁺. ¹H NMR (CDCl₃): 8.48 (s, 1H), 8.08 (d, J=8.1, 1H), 7.54 (t, J=7.2, 1H), 7.47 (d, J=8.5, 1H), 7.32-7.26 (m, 4H), 7.11 (d, J=7.5, 1H), 7.02 (s, 1H), 6.96 (d, J=8.9, 2H), 6.89 (dd, J=8.0, 1.9, 1H), 3.66-3.52 (m, 6H), 2.65 (t, J=6.7, 2H), 2.45 (s, 2H), 1.79-1.61 (m, 6H).

### Example 3

### 2-[3-(4-Chloro-phenoxy)-benzyl]-2,8-diaza-spiro[4.5]decane-8-carboxylic acid pyridin-3-ylamide

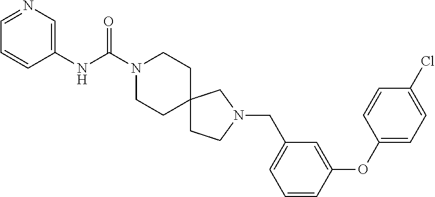

MS (ESI⁺): calcd for C₂₇H₂₉ClN₄O₂ m/z 476.20; found 477.2 (M+H)⁺. ¹H NMR (CDCl₃): 8.45 (s, 1H), 8.22 (d, J=3.6, 1H), 7.95 (d, J=8.2, 1H), 7.30-7.26 (m, 3H), 7.22-7.18 (m, 1H), 7.10 (d, J=7.6, 1H), 7.04 (s, 1H), 7.00 (s, 1H), 6.94 (d, J=8.9, 2H), 6.90 (dd, J=8.1, 1.8, 1H), 3.69 (s, 2H), 3.50-3.37 (m, 4H), 2.75 (s, 2H), 2.53 (s, 2H), 1.73 (t, J=6.8, 2H), 1.63-1.59 (m, 4H).

### Example 4

### 2-[3-(4-Chloro-phenoxy)-benzyl]-2,7-diaza-spiro[3.5]nonane-7-carboxylic acid pyridin-3-ylamide

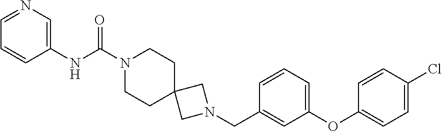

MS (ESI⁺): calcd for C₂₆H₂₇ClN₄O₂ m/z 462.18; found 463.2 (M+H)⁺. ¹H NMR (CDCl₃): 8.40 (d, J=2.6, 1H), 8.21 (dd, J=4.7, 1.3, 1H), 7.93 (dd, J=8.3, 3.9, 1H), 7.29-7.25 (m, 3H), 7.21-7.17 (m, 2H), 7.03 (d, J=7.6, 1H), 6.95-6.91 (m, 3H), 6.86 (dd, J=8.0, 2.1, 1H), 3.62 (s, 2H), 3.42-3.38 (m, 4H), 3.05 (s, 4H), 1.78-1.74 (m, 4H).

### Example 5

### 2-[3-(4-Chloro-phenoxy)-benzyl]-2,7-diaza-spiro[3.5]nonane-7-carboxylic acid benzo[d]isoxazol-3-ylamide

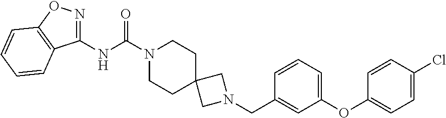

MS (ESI⁺): calcd for C₂₈H₂₇ClN₄O₃ m/z 502.18; found 503.2 (M+H)⁺. ¹H NMR (CDCl₃): 8.91 (s, 1H), 8.04 (d, J=8.1, 1H), 7.52 (t, J=8.2, 1H), 7.45 (d, J=8.5, 1H), 7.30-7.25 (m, 4H), 7.05 (d, J=7.6, 1H), 6.96-6.92 (m, 3H), 6.87 (dd, J=7.9, 2.1, 1H), 3.64 (s, 2H), 3.59-3.55 (m, 4H), 3.09 (s, 4H), 1.87-1.83 (m, 4H).

### Example 6

### 2-[3-(4-Chloro-phenoxy)-benzyl]-2,7-diaza-spiro[3.5]nonane-7-carboxylic acid (6-1,2,3]triazol-2-yl-pyridin-3-yl)-amide

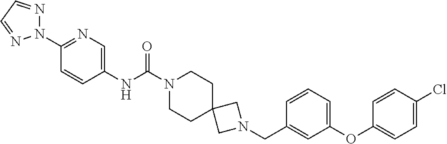

MS (ESI⁺): calcd for C₂₈H₂₈ClN₇O₂ m/z 529.20; found 530.2 (M+H)⁺. ¹H NMR (CDCl₃): 8.37 (d, J=2.5, 1H), 8.23 (dd, J=9.0, 2.6, 1H), 7.96 (d, J=8.9, 1H), 7.83 (s, 2H), 7.32 (s, 1H), 7.29-7.24 (m, 3H), 7.02 (d, J=7.6, 1H), 6.94-6.90 (m, 3H), 6.86 (dd, J=8.0, 1.8, 1H), 3.61 (s, 2H), 3.44-3.41 (m, 4H), 3.04 (s, 4H), 1.78-1.74 (m, 4H).

### Example 7

### 1-[3-(4-Chloro-phenoxy)-benzyl]-1,7-diaza-spiro[4.4]nonane-7-carboxylic acid pyridin-3-ylamide

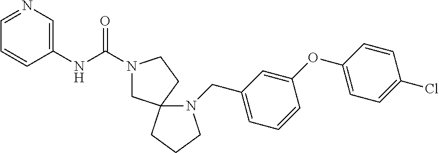

MS (ESI⁺): calcd for C₂₈H₂₇ClN₄O₂ m/z 462.18; found 463.2 (M+H)⁺. ¹H NMR (CDCl₃): 8.56 (d, J=1.8, 1H), 8.23 (d, J=4.2, 1H), 8.13 (d, J=8.4, 1H), 7.30-7.22 (m, 3H), 7.09 (d, J=7.7, 1H), 7.03-7.01 (m, 1H), 6.92 (d, J=9.0, 2H), 6.86 (dd, J=8.1, 1.7, 1H), 6.81 (s, 1H), 3.78-3.66 (m, 3H), 3.62 (d, J=10.2, 1H), 3.53-3.45 (m, 1H), 3.35 (d, J=10.2, 1H), 2.80-2.68 (m, 2H), 2.28-2.17 (m, 1H), 2.02-1.77 (m, 5H).

### Example 8

### 1-[3-(4-Chloro-phenoxy)-benzyl]-1,7-diaza-spiro[4.4]nonane-7-carboxylic acid (6-[1,2,3]triazol-2-yl-pyridin-3-yl)-amide

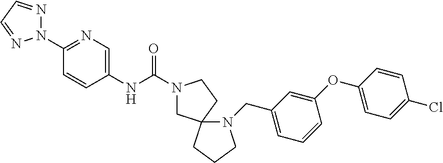

MS (ESI⁺): calcd for C₂₈H₂₈ClN₇O₂ m/z 529.20; found 530.2 (M+H)⁺. ¹H NMR (CDCl₃): 8.43 (d, J=2.4, 1H), 8.35 (dd, J=8.9, 2.6, 1H), 7.99 (d, J=8.9, 1H), 7.84 (s, 2H), 7.30-7.23 (m, 3H), 7.07 (d, J=7.7, 1H), 7.01-6.99 (m, 1H), 6.92 (d, J=9.0, 2H), 6.84 (dd, J=8.1, 1.7, 1H), 6.69 (s, 1H), 3.75-3.60 (m, 3H), 3.52-3.45 (m, 2H), 3.33 (d, J=9.8, 1H), 2.74-2.62 (m, 2H), 2.21-2.11 (m, 1H), 1.98-1.72 (m, 5H).

### Example 9

### 1-[3-(4-Chloro-phenoxy)-benzyl]-1,7-diaza-spiro[4.4]nonane-7-carboxylic acid (1H-pyrrolo[2,3-b]pyridin-5-yl)-amide

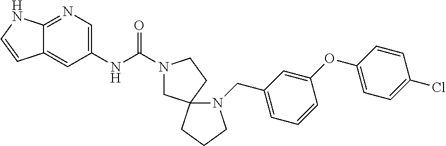

MS (ESI⁺): calcd for C₂₈H₂₈ClN₅O₂ m/z 501.19; found 502.2 (M+H)⁺. ¹H NMR (CDCl₃): 10.08 (s, 1H), 8.24 (d, J=2.3, 1H), 8.10 (d, J=2.3, 1H), 7.29-7.24 (m, 4H), 7.08 (d, J=7.7, 1H), 7.02-7.00 (m, 1H), 6.92 (d, J=9.0, 2H), 6.84 (dd, J=8.1, 1.7, 1H), 6.42 (dd, J=3.5, 1.9, 1H), 6.28 (s, 1H), 3.72-3.60 (m, 3H), 3.51-3.42 (m, 2H), 3.31 (d, J=9.8, 1H), 2.73-2.62 (m, 2H), 2.18-2.10 (m, 1H), 1.98-1.91 (m, 1H), 1.89-1.70 (m, 4H).

### Example 10

### 7-[3-(4-Chloro-phenoxy)-benzyl]-2,7-diaza-spiro[4.4]nonane-2-carboxylic acid pyridin-3-ylamide

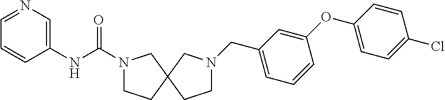

MS (ESI⁺): calcd for C₂₆H₂₇ClN₄O₂ m/z 462.18; found 463.5 (M+H)⁺. ¹H NMR (CDCl₃): 8.44 (d, J=2.2, 1H), 8.25 (dd, J=4.7, 1.4, 1H), 8.06 (dd, J=8.4, 4.1, 1H), 7.30-7.25 (m, 3H), 7.22 (dd, J=8.3, 4.7, 1H), 7.07 (d, J=7.6, 1H), 7.00-6.98 (m, 1H), 6.93 (d, J=9.0, 2H), 6.87 (dd, J=7.7, 2.9, 1H), 6.30 (s, 1H), 3.63-3.51 (m, 3H), 3.46 (d, J=9.6, 2H), 3.36 (d, J=9.4, 1H), 2.73-2.66 (m, 1H), 2.64-2.52 (m, 2H), 2.44 (d, J=9.2, 1H), 2.02-1.77 (m, 4H).

### Example 11

### 7-[3-(4-Chloro-phenoxy)-benzyl]-2,7-diaza-spiro[4.4]nonane-2-carboxylic acid (6-[1,2,3]triazol-2-yl-pyridin-3-yl)-amide

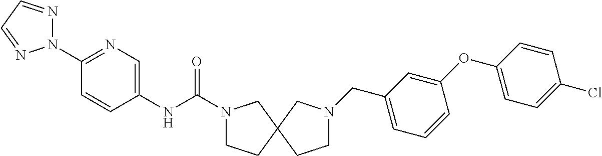

MS (ESI⁺): calcd for C₂₈H₂₈ClN₇O₂ m/z 529.20; found 530.6 (M+H)⁺. ¹H NMR (CDCl₃): 8.41 (d, J=2.2, 1H), 8.35-8.32 (m, 1H), 7.99 (d, J=8.9, 1H), 7.85 (s, 2H), 7.30-7.25 (m, 3H), 7.07 (d, J=7.7, 1H), 6.99 (s, 1H), 6.93 (d, J=9.0, 2H), 6.87 (dd, J=8.1, 1.7, 1H), 6.56 (s, 1H), 3.63-3.55 (m, 3H), 3.51-3.45 (m, 2H), 3.38 (d, J=9.4, 1H), 2.73-2.66 (m, 1H), 2.63-2.52 (m, 2H), 2.43 (d, J=9.2, 1H), 2.02-1.76 (m, 4H).

### Example 12

### 7-[3-(4-Chloro-phenoxy)-benzyl]-2,7-diaza-spiro[4.4]nonane-2-carboxylic acid (1H-pyrrolo[2,3-b]pyridin-5-yl)-amide

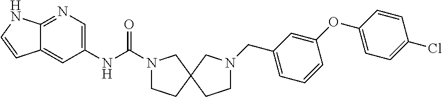

MS (ESI⁺): calcd for C₂₈H₂₈ClN₅O₂ m/z 501.19; found 502.5 (M+H)⁺. ¹H NMR (CDCl₃): 10.24 (s, 1H), 8.21 (d, J=2.3, 1H), 8.09 (d, J=2.3, 1H), 7.29-7.25 (m, 4H), 7.08 (d, J=7.6, 1H), 7.00-6.98 (m, 1H), 6.92 (d, J=9.0, 2H), 6.87 (dd, J=7.8, 2.8, 1H), 6.41 (d, J=3.5, 1H), 6.26 (s, 1H), 3.63-3.50 (m, 3H), 3.46 (d, J=10.8, 2H), 3.35 (d, J=9.4, 1H), 2.73-2.65 (m, 1H), 2.63-2.54 (m, 2H), 2.44 (d, J=9.2, 1H), 1.98-1.76 (m, 4H).

### Example 13

### 9-[3-(4-Chloro-phenoxy)-benzyl]-3,9-diaza-spiro[5.5]undecane-3-carboxylic acid pyridin-3-ylamide

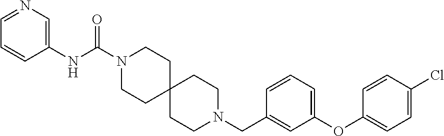

MS (ESI⁺): calcd for C₂₈H₃₁ClN₄O₂ m/z 490.21; found 491.6 (M+H)⁺. ¹H NMR (CDCl₃): 8.41 (d, J=2.6 Hz, 1H), 8.25 (dd, J=4.7, 1.4, 1H), 8.00-7.95 (m, 1H), 7.32-7.24 (m, 3H), 7.21 (dd, J=8.4, 4.7, 1H), 7.07 (d, J=7.6, 1H), 7.01-6.98 (m, 1H), 6.92 (d, J=8.8, 2H), 6.87 (dd, J=8.1, 1.8, 1H), 6.60 (s, 1H), 3.51-3.41 (m, 6H), 2.46-2.33 (m, 4H), 1.58-1.48 (m, 8H).

### Example 14

### 9-[3-(4-Chloro-phenoxy)-benzyl]-3,9-diaza-spiro[5.5]undecane-3-carboxylic acid (6-[1,2,3]triazol-2-yl-pyridin-3-yl)-amide

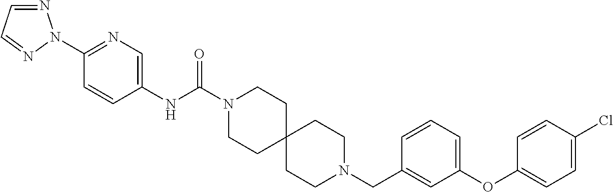

MS (ESI⁺): calcd for C₃₀H₃₂ClN₇O₂ m/z 557.23; found 558.6 (M+H)⁺. ¹H NMR (CDCl₃): 8.38 (d, J=2.5, 1H), 8.26 (dd, J=8.9, 2.6, 1H), 7.97 (d, J=9.0, 1H), 7.83 (s, 2H), 7.30-7.24 (m, 4H), 7.07 (d, J=7.6, 1H), 7.00-6.98 (m, 1H), 6.92 (d, J=8.9, 2H), 6.86 (dd, J=8.1, 1.7, 1H), 3.51-3.42 (m, 6H), 2.44-2.33 (m, 4H), 1.56-1.46 (m, 8H).

### Example 15

### 9-[3-(4-Chloro-phenoxy)-benzyl]-3,9-diaza-spiro[5.5]undecane-3-carboxylic acid (1H-pyrrolo[2,3-b]pyridin-5-yl)-amide

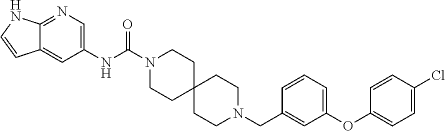

MS (ESI⁺): calcd for C₃₀H₃₂ClN₅O₂ m/z 529.22; found 530.6 (M+H)⁺. ¹H NMR (CDCl₃): 8.18 (d, J=2.3, 1H), 8.01 (d, J=2.3, 1H), 7.32-7.23 (m, 4H), 7.07 (d, J=7.6, 1H), 7.01-6.99 (m, 1H), 6.93 (d, J=8.9, 2H), 6.87 (dd, J=8.1, 1.7, 1H), 6.41 (d, J=3.5, 1H), 3.48 (s, 2H), 3.45-3.38 (m, 4H), 2.44-2.34 (m, 4H), 1.55-1.44 (m, 8H).

### Example 16

### 2-(3-(4-chlorophenoxy)benzyl)-N-(pyridin-3-yl)-2,7-diazaspiro[4.5]decane-7-carboxamide.

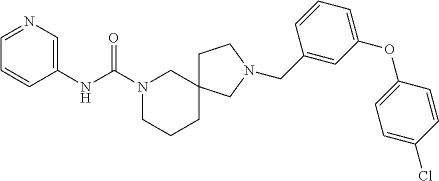

MS (ESI⁺): calcd for C₂₇H₂₉ClN₄O₂ m/z 476.20; found 477.2 (M+H)⁺. ¹H NMR (CDCl₃): 8.40 (d, J=2.2, 1H), 8.23 (dd, J=4.7, 1.4, 1H), 7.94-7.91 (m, 1H), 7.28-7.24 (m, 2H), 7.22-7.16 (m, 2H), 7.05 (d, J=7.7, 1H), 6.97-6.93 (m, 2H), 6.90 (d, J=9.0, 2H), 6.84-6.81 (m, 1H), 3.84 (d, J=12.9, 1H), 3.59 (dd, J=27.9, 13.3, 2H), 3.45 (s, 1H), 3.16-3.07 (m, 2H), 2.95-2.90 (s, 1H), 2.78 (d, J=9.5, 1H), 2.38 (dd, J=16.5, 9.1, 1H), 2.06 (d, J=9.5, 1H), 1.67-1.45 (m, 6H).

### Example 17

### N-(6-(2H-1,2,3-triazol-2-yl)pyridin-3-yl)-2-(3-(4-chlorophenoxy)benzyl)-2,7-diazaspiro[4.5]decane-7-carboxamide.

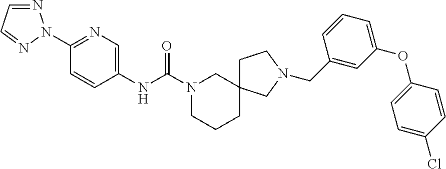

MS (ESI⁺): calcd for C₂₉H₃₀ClN₇O₂ m/z 543.22; found 544.2 (M+H)⁺. ¹H NMR (CDCl₃): 8.44 (s, 1H), 8.20 (dd, J=8.9, 2.6, 1H), 7.94 (d, J=8.9, 1H), 7.85 (s, 2H), 7.27-7.21 (m, 3H), 7.07 (d, J=7.6, 1H), 6.96 (s, 1H), 6.90 (d, J=9.0, 2H), 6.84 (dd, J=8.1, 1.8, 1H), 3.90 (d, J=12.7, 1H), 3.66 (s, 2H), 3.45 (s, 1H), 3.15-3.00 (m, 3H), 2.90 (d, J=7.1, 1H), 2.46 (d, J=7.1, 1H), 2.11 (d, J=9.5, 1H), 1.74-1.58 (m, 6H).

### Example 18

### 8-(3-(4-chlorophenoxy)benzyl)-N-(pyridin-3-yl)-2,8-diazaspiro[4.5]decane-2-carboxamide.

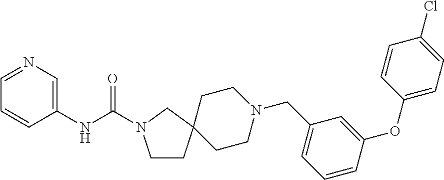

MS (ESI⁺): calcd for C₂₇H₂₉ClN₄O₂ m/z 476.20; found 477.2 (M+H)⁺. ¹H NMR (CDCl₃): 8.60 (s, 1H), 8.24 (dd, J=4.7, 1.4, 1H), 8.09-8.06 (m, 1H), 7.30-7.26 (m, 3H), 7.23 (dd, J=8.4, 4.7, 1H), 7.10 (d, J=7.6, 1H), 7.00 (s, 1H), 6.93-6.88 (m, 3H), 3.76-3.51 (m, 4H), 3.48 (s, 2H), 3.33 (d, J=9.4, 1H), 2.65-2.23 (m, 3H), 1.92 (s, 1H), 1.80-1.70 (m, 3H), 1.63 (s, 1H), 1.49-1.41 (m, 1H).

### Example 19

### N-(6-(2H-1,2,3-triazol-2-yl)pyridin-3-yl)-8-(3-(4-chlorophenoxy)benzyl)-2,8-diazaspiro[4.5]decane-2-carboxamide.

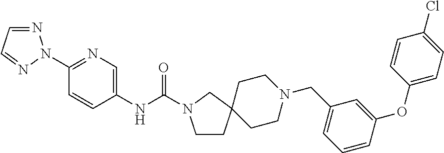

MS (ESI⁺): calcd for C₂₉H₃₀ClN₇O₂ m/z 543.22; found 544.2 (M+H)⁺. ¹H NMR (CDCl₃): 8.44 (s, 1H), 8.34 (dd, J=8.9, 2.6, 1H), 7.98 (d, J=8.9, 1H), 7.85 (s, 2H), 7.28-7.23 (m, 3H), 7.07 (d, J=7.5, 1H), 6.99-6.98 (m, 1H), 6.91 (d, J=8.9, 2H), 6.85 (d, J=8.0, 1H), 3.53 (s, 6H), 3.28 (d, J=8.3, 1H), 2.41-2.12 (m, 3H), 1.93 (s, 1H), 1.78-1.53 (m, 4H), 1.47-1.39 (m, 1H).

### Example 20

### 9-(3-(4-chlorophenoxy)benzyl)-N-(pyridin-3-yl)-2,9-diazaspiro[5.5]undecane-2-carboxamide.

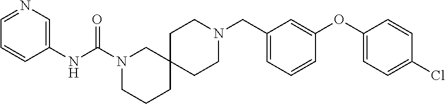

MS (ESI⁺): calcd for C₂₈H₃₁ClN₄O₂ m/z 490.21; found 491.2 (M+H)⁺. ¹H NMR (CDCl₃): 8.73 (s, 1H), 8.19 (d, J=4.4, 1H), 8.05 (d, J=8.3, 1H), 7.33-7.24 (m, 4H), 7.20-7.15 (m, 2H), 6.94-6.87 (m, 3H), 3.86 (s, 2H), 3.56-3.47 (m, 4H), 2.97-2.66 (m, 4H), 1.74-1.57 (m, 6H), 1.50-1.46 (m, 2H).

### Example 21

### N-(6-(2H-1,2,3-triazol-2-yl)pyridin-3-yl)-9-(3-(4-chlorophenoxy)benzyl)-2,9-diazaspiro[5.5]undecane-2-carboxamide.

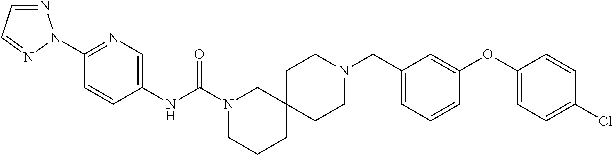

MS (ESI⁺): calcd for C₃₀H₃₂ClN₇O₂ m/z 557.23; found 558.3 (M+H)⁺. ¹H NMR (CDCl₃): 8.57 (s, 1H), 8.30 (dd, J=8.9, 2.6, 1H), 7.95 (d, J=8.9, 1H), 7.84 (s, 2H), 7.30-7.24 (m, 3H), 7.19-7.14 (m, 1H), 7.06 (s, 1H), 6.91 (d, J=9.0, 2H), 6.87 (dd, J=8.1, 1.6, 1H), 3.68 (s, 2H), 3.52-3.48 (m, 2H), 3.43 (s, 2H), 2.64 (s, 4H), 1.68-1.54 (m, 6H), 1.51-1.45 (m, 2H).

### Example 22

### 2-(3-(4-chlorophenoxy)benzyl)-N-(pyridin-3-yl)-2,9-diazaspiro[5.5]undecane-9-carboxamide.

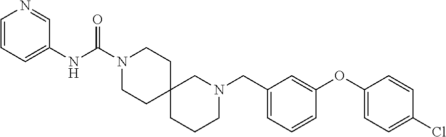

MS (ESI⁺): calcd for C₂₈H₃₁ClN₄O₂ m/z 490.21; found (M+H)⁺. ¹H NMR (CDCl₃): 8.41 (d, J=2.4, 1H), 8.24 (dd, J=4.7, 1.4, 1H), 7.99-7.94 (m, 1H), 7.31-7.23 (m, 3H), 7.20 (dd, J=8.4, 4.7, 1H), 7.05 (d, J=7.6, 1H), 6.99-6.96 (m, 1H), 6.93 (d, J=9.0, 2H), 6.86 (dd, J=8.1, 1.7, 1H), 6.58 (s, 1H), 3.48-3.38 (m, 4H), 3.33-3.24 (m, 2H), 2.41 (s, 2H), 2.15 (s, 2H), 1.65-1.45 (m, 6H), 1.41-1.32 (m, 2H).

### Example 23

### N-(6-(2H-1,2,3-triazol-2-yl)pyridin-3-yl)-2-(3-(4-chlorophenoxy)benzyl)-2,9-diazaspiro[5.5]undecane-9-carboxamide.

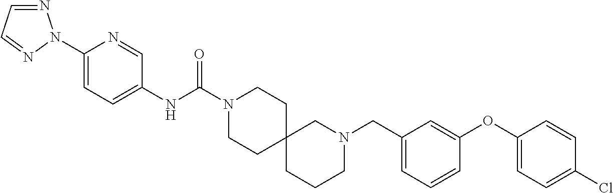

MS (ESI⁺): calcd for C₃₀H₃₂ClN₇O₂ m/z 557.23; found (M+H)⁺. ¹H NMR (CDCl₃): 8.36 (d, J=2.2, 1H), 8.25 (dd, J=8.9, 2.7, 1H), 7.97 (d, J=8.9, 1H), 7.84 (s, 2H), 7.30-7.24 (m, 3H), 7.05 (d, J=7.7, 1H), 6.98-6.96 (m, 1H), 6.93 (d, J=9.0, 2H), 6.89-6.84 (m, 2H), 3.50-3.38 (m, 4H), 3.35-3.26 (m, 2H), 2.40 (s, 2H), 2.14 (s, 2H), 1.64-1.44 (m, 6H), 1.39-1.32 (m, 2H).

### Example 24

### 8-(3-(4-chlorophenoxy)benzyl)-N-(pyridin-3-yl)-2,8-diazaspiro[5.5]undecane-2-carboxamide.

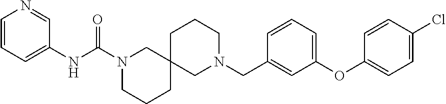

MS (ESI⁺): calcd for C₂₈H₃₁ClN₄O₂ m/z 490.21; found 491.2 (M+H)⁺. ¹H NMR (CDCl₃): 8.39 (s, 1H), 8.21 (dd, J=4.7, 1.4, 1H), 7.94 (d, J=8.3, 1H), 7.54 (s, 1H), 7.30-7.20 (m, 3H), 7.16 (dd, J=8.3, 4.7, 1H), 6.95 (d, J=6.5, 1H), 6.90 (d, J=8.9, 2H), 6.86 (d, J=8.0, 1H), 4.17 (d, J=11.1, 1H), 4.03 (d, J=13.5, 1H), 3.74 (d, J=13.8, 1H), 3.51 (d, J=12.6, 1H), 2.96-2.74 (m, 4H), 2.12-1.95 (m, 1H), 1.85-1.69 (m, 2H), 1.65-1.34 (m, 6H), 1.16 (t, J=10.8, 1H).

### Example 25

### N-(6-(2H-1,2,3-triazol-2-yl)pyridin-3-yl)-8-(3-(4-chlorophenoxy)benzyl)-2,8-diazaspiro[5.5]undecane-2-carboxamide.

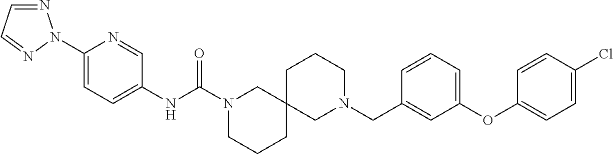

MS (ESI⁺): calcd for C₃₀H₃₂ClN₇O₂ m/z 557.23; found 558.2 (M+H)⁺. ¹H NMR (CDCl₃): 8.37 (d, J=2.4, 1H), 8.17 (dd, J=8.9, 2.6, 1H), 7.93 (d, J=8.9, 1H), 7.85 (s, 2H), 7.80 (s, 1H), 7.29-7.20 (m, 3H), 6.95 (d, J=7.5, 1H), 6.90 (d, J=9.0, 2H), 6.88-6.83 (m, 2H), 4.20 (d, J=12.0, 1H), 4.06 (d, J=13.3, 1H), 3.73 (d, J=13.8, 1H), 3.51 (d, J=13.8, 1H), 2.93-2.76 (m, 4H), 2.11-2.02 (m, 1H), 1.84-1.72 (m, 2H), 1.62-1.35 (m, 6H), 1.20-1.11 (m, 1H).

### Example 26

### 2-(3-(4-chlorophenoxy)benzyl)-N-(pyridin-3-yl)-2,6-diazaspiro[3.5]nonane-6-carboxamide.

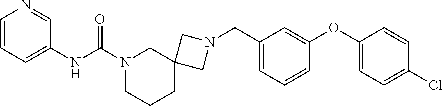

MS (ESI⁺): calcd for C₂₆H₂₇ClN₄O₂ m/z 462.18; found 463.2 (M+H)⁺. ¹H NMR (CDCl₃): 8.51 (d, J=2.2, 1H), 8.19 (dd, J=4.7, 1.5, 1H), 8.04-8.00 (m, 1H), 7.76 (s, 1H), 7.30-7.23 (m, 3H), 7.17 (dd, J=8.4, 4.7, 1H), 7.01 (d, J=7.6, 1H), 6.93-6.89 (m, 3H), 6.88-6.84 (m, 1H), 3.62 (d, J=5.1, 4H), 3.46-3.42 (m, 2H), 3.22 (d, J=8.3, 2H), 2.85 (d, J=8.2, 2H), 1.73-1.68 (m, 2H), 1.55-1.48 (m, 2H).

### Example 27

### N-(6-(2H-1,2,3-triazol-2-yl)pyridin-3-yl)-2-(3-(4-chlorophenoxy)benzyl)-2,6-diazaspiro[3.5]nonane-6-carboxamide.

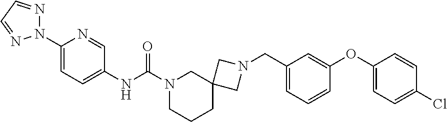

MS (ESI⁺): calcd for C₂₈H₂₈ClN₇O₂ m/z 529.20; found 530.2 (M+H)⁺. ¹H NMR (CDCl₃): 8.49 (dd, J=2.6, 0.4, 1H), 8.24 (dd, J=8.9, 2.7, 1H), 7.95 (d, J=8.9, 1H), 7.84 (s, 2H), 7.77 (s, 1H), 7.29-7.24 (m, 3H), 7.00 (d, J=7.8, 1H), 6.93-6.89 (m, 3H), 6.87-6.84 (m, 1H), 3.62 (d, J=9.4, 4H), 3.48-3.43 (m, 2H), 3.21 (d, J=8.2, 2H), 2.86 (d, J=8.2, 2H), 1.74-1.67 (m, 2H), 1.55-1.49 (m, 2H).

### Example 28

### 7-(3-(4-chlorophenoxy)benzyl)-N-(imidazo[1,2-b]pyridazin-3-yl)-2,7-diazaspiro[3.5]nonane-2-carboxamide.

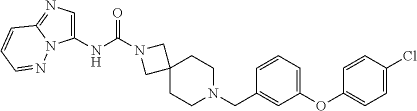

MS (ESI⁺): calcd for C₂₇H₂₇ClN₆O₂ m/z 502.19; found 503.2 (M+H)⁺. ¹H NMR (CDCl₃): 8.26 (dd, J=4.4, 1.5, 1H), 8.00 (s, 1H), 7.89 (dd, J=9.2, 1.5, 1H), 7.31-7.24 (m, 3H), 7.07 (d, J=7.7, 1H), 7.01-6.98 (m, 1H), 6.95-6.85 (m, 5H), 3.83 (s, 4H), 3.45 (s, 2H), 2.37 (s, 4H), 1.82 (t, J=5.4, 4H).

### Example 29

### 7-(3-(4-chlorophenoxy)benzyl)-N-(imidazo[1,2-a]pyridin-3-yl)-2,7-diazaspiro[3.5]nonane-2-carboxamide.

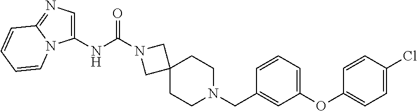

MS (ESI⁺): calcd for C₂₈H₂₈ClN₅O₂ m/z 501.19; found 502.2 (M+H)⁺. ¹H NMR (CDCl₃): 7.91 (d, J=6.9, 1H), 7.52 (d, J=9.1, 1H), 7.41 (s, 1H), 7.30-7.23 (m, 3H), 7.20-7.14 (m, 1H), 7.04 (d, J=7.6, 1H), 6.99-6.96 (m, 1H), 6.92 (d, J=9.0, 2H), 6.88-6.84 (m, 1H), 6.83-6.78 (m, 1H), 6.56 (s, 1H), 3.59 (s, 4H), 3.41 (s, 2H), 2.29 (s, 4H), 1.71 (t, J=5.4, 4H).

### Example 30

### 2-(3-(4-chlorophenoxy)benzyl)-N-(4-chloropyridin-3-yl)-2,6-diazaspiro[3.5]nonane-6-carboxamide.

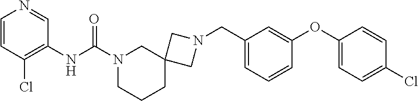

MS (ESI⁺): calcd for C₂₆H₂₆Cl₂N₄O₂ m/z 496.14; found 497.2 (M+H)⁺. ¹H NMR (CDCl₃): 9.28 (s, 1H), 8.18 (d, J=5.2, 1H), 7.30-7.22 (m, 4H), 7.03 (d, J=7.7, 1H), 6.99 (s, 1H), 6.95-6.88 (m, 3H), 6.85 (dd, J=8.1, 1.7, 1H), 3.64 (d, J=6.0, 4H), 3.48-3.43 (m, 2H), 3.20 (d, J=8.0, 2H), 2.91 (d, J=7.9, 2H), 1.81-1.73 (m, 2H), 1.62-1.54 (m, 2H).

### Example 31

### 9-(3-(4-chlorophenoxy)benzyl)-N-(4-chloropyridin-3-yl)-3,9-diazaspiro[5.5]undecane-3-carboxamide.

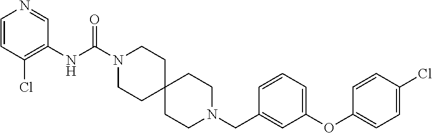

MS (ESI⁺): calcd for C₂₈H₃₀Cl₂N₄O₂ m/z 524.17; found 525.2 (M+H)⁺. ¹H NMR (CDCl₃): 9.37 (s, 1H), 8.17 (d, J=5.2, 1H), 7.32-7.23 (m, 4H), 7.08 (d, J=7.7, 1H), 7.02-6.98 (m, 1H), 6.92 (d, J=9.0, 2H), 6.87 (dd, J=8.1, 1.7, 1H), 6.84 (s, 1H), 3.54-3.45 (m, 6H), 2.48-2.39 (m, 4H), 1.61-1.49 (m, 8H).

### Example 32

### 2-(3-(4-chlorophenoxy)benzyl)-N-(quinolin-3-yl)-2,6-diazaspiro[3.5]nonane-6-carboxamide.

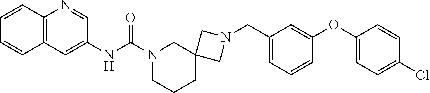

MS (ESI⁺): calcd for C₃₀H₂₉ClN₄O₂ m/z 512.20; found 513.2 (M+H)⁺. ¹H NMR (CDCl₃): 9.03 (s, 1H), 8.61 (s, 1H), 7.94 (d, J=8.0, 1H), 7.82 (dd, J=8.4, 0.8, 1H), 7.64 (t, J=7.0, 1H), 7.56 (t, J=7.0, 1H), 7.27-7.20 (m, 3H), 7.09 (s, 1H), 7.02 (d, J=7.6, 1H), 6.94-6.91 (m, 1H), 6.90-6.82 (m, 3H), 3.68-3.60 (m, 4H), 3.51-3.43 (m, 3H), 3.23 (d, J=8.0, 2H), 2.90 (d, J=8.0, 2H), 1.79-1.72 (m, 2H), 1.60-1.52 (m, 2H).

### Example 33

### 9-(3-(4-chlorophenoxy)benzyl)-N-(quinolin-3-yl)-3,9-diazaspiro[5.5]undecane-3-carboxamide.

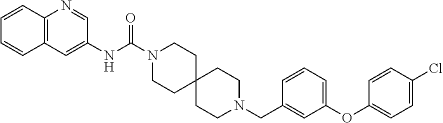

MS (ESI⁺): calcd for C₃₂H₃₃ClN₄O₂ m/z 540.23; found 541.2 (M+H)⁺. ¹H NMR (CDCl₃): 9.03 (s, 1H), 8.59 (s, 1H), 7.94 (d, J=8.1, 1H), 7.80 (dd, J=8.4, 0.8, 1H), 7.68 (t, J=7.0, 1H), 7.58 (t, J=7.0, 1H), 7.31-7.24 (m, 3H), 7.08 (d, J=7.7, 1H), 7.02-7.00 (m, 1H), 6.93 (d, J=9.0, 2H), 6.87 (dd, J=8.1, 1.6, 1H), 6.73 (s, 1H), 3.52-3.43 (m, 6H), 2.45-2.37 (m, 4H), 1.60-1.47 (m, 8H).

### Example 34

### 2-(3-(4-chlorophenoxy)benzyl)-N-(imidazo[1,2-a]pyridin-3-yl)-2,6-diazaspiro[3.5]nonane-6-carboxamide.

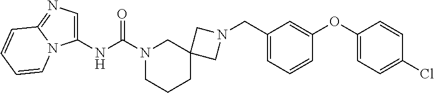

MS (ESI⁺): calcd for C₂₈H₂₈ClN₅O₂ m/z 501.19; found 502.2 (M+H)⁺. ¹H NMR (CDCl₃): 7.84 (d, J=6.9, 1H), 7.52 (d, J=9.1, 1H), 7.37 (s, 1H), 7.28-7.21 (m, 4H), 7.15-7.10 (m, 1H), 7.01 (d, J=7.7, 1H), 6.94-6.83 (m, 4H), 6.78-6.73 (m, 1H), 3.62 (d, J=3.8, 4H), 3.47-3.41 (m, 2H), 3.20 (d, J=7.9, 2H), 2.89 (d, J=7.9, 2H), 1.79-1.71 (m, 2H), 1.59-1.50 (m, 2H).

### Example 35

### 6-(3-(4-chlorophenoxy)benzyl)-N-(pyridin-3-yl)-2,6-diazaspiro[3.3]heptane-2-carboxamide.

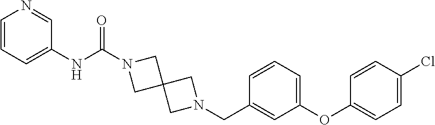

MS (ESI⁺): calcd for C₂₄H₂₃ClN₄O₂ m/z 434.15; found 435.2 (M+H)⁺. ¹H NMR (CDCl₃): 8.43 (d, J=2.6, 1H), 8.24 (dd, J=4.7, 1.4, 1H), 8.03-7.98 (m, 1H), 7.31-7.25 (m, 3H), 7.20 (dd, J=8.4, 4.7, 1H), 7.00 (d, J=7.6, 1H), 6.95-6.90 (m, 3H), 6.88 (dd, J=7.8, 2.1, 1H), 6.46 (s, 1H), 4.12 (s, 4H), 3.54 (s, 2H), 3.35 (s, 4H).

### Example 36

### 6-(3-(4-chlorophenoxy)benzyl)-N-(4-chloropyridin-3-yl)-2,6-diazaspiro[3.3]heptane-2-carboxamide.

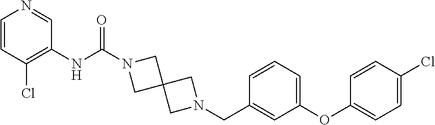

MS (ESI⁺): calcd for C₂₄H₂₂Cl₂N₄O₂ m/z 468.11; found 469.1 (M+H)⁺. ¹H NMR (CDCl₃): 9.42 (s, 1H), 8.18 (d, J=5.2, 1H), 7.34-7.23 (m, 3H), 7.01 (d, J=7.5, 1H), 6.97-6.85 (m, 4H), 6.37 (s, 1H), 4.18 (s, 4H), 3.55 (s, 2H), 3.37 (s, 4H).

### Example 37

### 7-[3-(4-Chloro-phenoxy)-benzyl]-2,7-diaza-spiro[3.5]nonane-2-carboxylic acid pyridin-3-ylamide

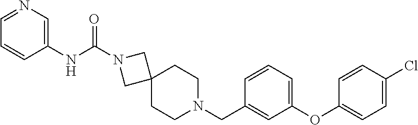

MS (ESI⁺): calcd for C₂₆H₂₇ClN₄O₂ m/z 462.18; found 463.2 (M+H)⁺. ¹H NMR (CDCl₃): 8.44 (d, J=2.5, 1H), 8.22 (dd, J=4.7, 1.4, 1H), 8.06-8.02 (m, 1H), 7.29-7.24 (m, 3H), 7.20 (dd, J=8.4, 4.7, 1H), 7.06 (d, J=7.6, 1H), 6.99-6.97 (m, 1H), 6.92 (d, J=9.0, 2H), 6.87 (dd, J=8.1, 2.4, 1H), 3.74 (s, 4H), 3.43 (s, 2H), 2.33 (s, 4H), 1.79-1.75 (m, 4H).

### Example 38

### 7-[3-(4-Chloro-phenoxy)-benzyl]-2,7-diaza-spiro[3.5]nonane-2-carboxylic acid benzo[d]isoxazol-3-ylamide

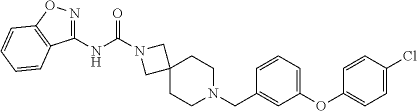

MS (ESI⁺): calcd for C₂₈H₂₇ClN₄O₃ m/z 502.18; found 503.2 (M+H)⁺. ¹H NMR (CDCl₃): 8.25 (d, J=8.1, 1H), 7.52 (t, J=7.8, 1H), 7.44 (d, J=8.4, 1H), 7.30-7.25 (m, 4H), 7.07 (d, J=7.5, 1H), 7.01-6.98 (m, 1H), 6.93 (d, J=8.9, 2H), 6.87 (dd, J=8.0, 2.3, 1H), 3.87 (s, 4H), 3.45 (s, 2H), 2.36 (s, 4H), 1.84-1.80 (m, 4H).

### Example 39

### 7-[3-(4-Chloro-phenoxy)-benzyl]-2,7-diaza-spiro[3.5]nonane-2-carboxylic acid (6-[1,2,3]triazol-2-yl-pyridin-3-yl)-amide

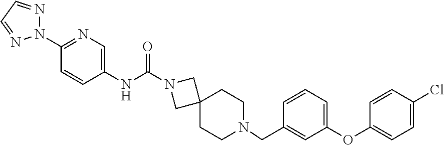

MS (ESI⁺): calcd for C₂₈H₂₈ClN₇O₂ m/z 529.20; found 530.2 (M+H)⁺. ¹H NMR (CDCl₃): 8.39 (d, J=2.7, 1H), 8.33 (dd, J=8.9, 2.7, 1H), 7.99 (d, J=8.8, 1H), 7.85 (s, 2H), 7.30-7.25 (m, 4H), 7.06 (d, J=7.6, 1H), 7.00-6.98 (m, 1H), 6.93 (d, J=8.9, 2H), 6.87 (dd, J=8.1, 1.6, 1H), 3.77 (s, 4H), 3.44 (s, 2H), 2.34 (s, 4H), 1.81-1.77 (m, 4H).

### Example 40

### 7-[3-(4-Chloro-phenoxy)-benzyl]-2,7-diaza-spiro[3.5]nonane-2-carboxylic acid (1H-pyrrolo[2,3-b]pyridin-5-yl)-amide, trifluoroacetic acid salt

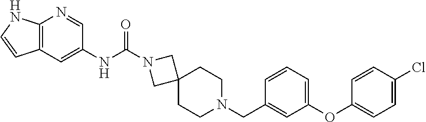

MS (ESI⁺): calcd for C₂₈H₂₈ClN₅O₂ m/z 501.19; found 502.2 (M+H)⁺. ¹H NMR (d₆-acetone): 8.65 (s, 1H), 8.59-8.54 (m, 1H), 7.72-7.66 (m, 1H), 7.48 (t, J=7.9, 1H), 7.45-7.37 (m, 3H), 7.32-7.29 (m, 1H), 7.16-7.11 (m, 1H), 7.10-7.05 (m, 2H), 6.70-6.63 (m, 1H), 4.42 (s, 2H), 3.99-3.84 (m, 4H), 3.64-3.50 (m, 2H), 3.19-3.05 (m, 2H), 2.30-2.14 (m, 4H).

### Example 41

### 7-[3-(4-Chloro-phenoxy)-benzyl]-2,7-diaza-spiro[3.5]nonane-2-carboxylic acid (4-[1,2,3]triazol-2-yl-phenyl)-amide, trifluoroacetic acid salt

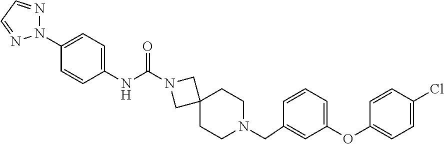

MS (ESI⁺): calcd for C₂₉H₂₉ClN₆O₂ m/z 528.20; found 529.2 (M+H)⁺. ¹H NMR (d₆-acetone): 7.98-7.90 (m, 4H), 7.76-7.71 (m, 2H), 7.47 (t, J=7.9, 1H), 7.43-7.38 (m, 3H), 7.32-7.29 (m, 1H), 7.14-7.10 (m, 1H), 7.09-7.05 (m, 2H), 4.38 (s, 2H), 3.88 (s, 4H), 3.51 (s, 2H), 3.12-3.00 (m, 2H), 2.23-2.13 (m, 4H).

### Example 42

### 7-[3-(4-Chloro-phenoxy)-benzyl]-2,7-diaza-spiro[3.5]nonane-2-carboxylic acid pyrimidin-2-ylamide, trifluoroacetic acid salt

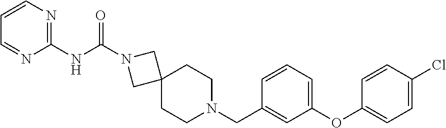

MS (ESI⁺): calcd for C₂₅H₂₆ClN₅O₂ m/z 463.18; found 464.2 (M+H)⁺. ¹H NMR (d₆-acetone): 8.69 (d, J=5.0, 2H), 7.47 (t, J=7.9, 1H), 7.42-7.37 (m, 3H), 7.31-7.28 (m, 1H), 7.23-7.19 (m, 1H), 7.14-7.10 (m, 1H), 7.08-7.04 (m, 2H), 4.42 (s, 2H), 4.09-3.92 (m, 4H), 3.61-3.49 (m, 2H), 3.22-3.07 (m, 2H), 2.22 (s, 4H).

### Example 43

### 7-[3-(4-Chloro-phenoxy)-benzyl]-2,7-diaza-spiro[3.5]nonane-2-carboxylic acid pyrimidin-4-ylamide, trifluoroacetic acid salt

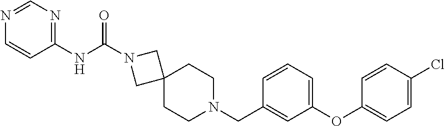

MS (ESI⁺): calcd for C₂₅H₂₆ClN₅O₂ m/z 463.18; found 464.2 (M+H)⁺. ¹H NMR (d₆-acetone): 8.96 (s, 1H), 8.70 (d, J=6.5, 1H), 8.31 (d, J=6.6, 1H), 7.49 (t, J=7.9, 1H), 7.44-7.38 (m, 3H), 7.31-7.28 (m, 1H), 7.15-7.11 (m, 1H), 7.10-7.05 (m, 2H), 4.43 (5, 2H), 4.06 (5, 4H), 3.64-3.51 (m, 2H), 3.24-3.08 (m, 2H), 2.36-2.15 (m, 4H).

### Example 44

### 7-[3-(4-Chloro-phenoxy)-benzyl]-2,7-diaza-spiro[3.5]nonane-2-carboxylic acid pyridazin-3-ylamide, trifluoroacetic acid salt

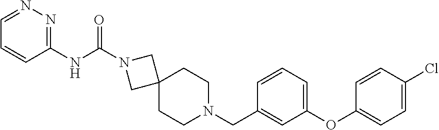

MS (ESI⁺): calcd for C₂₅H₂₆ClN₅O₂ m/z 463.18; found 464.2 (M+H)⁺. ¹H NMR (d₆-acetone): 8.89 (dd, J=4.5, 1.4, 1H), 8.53 (dd, J=9.4, 1.3, 1H), 8.01 (dd, J=9.4, 4.5, 1H), 7.46 (t, J=7.9, 1H), 7.41-7.35 (m, 3H), 7.31-7.27 (m, 1H), 7.12-7.07 (m, 1H), 7.04 (d, J=9.0, 4.55 (s, 2H), 4.16 (s, 4H), 3.69-3.59 (m, 4H), 2.06-1.99 (m, 4H).

### Example 45

### 7-[3-(4-Chloro-phenoxy)-benzyl]-2,7-diaza-spiro[3.5]nonane-2-carboxylic acid (6-pyrazol-1-yl-pyridin-3-yl)-amide, trifluoroacetic acid salt

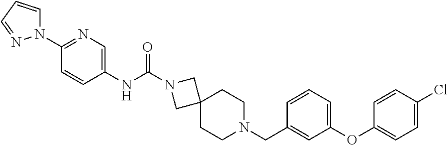

MS (ESI⁺): calcd for C₂₉H₂₉ClN₆O₂ m/z 528.20; found 529.2 (M+H)⁺. ¹H NMR (d₆-acetone): 8.55 (d, J=2.1, 1H), 8.49 (dd, J=2.5, 0.5, 1H), 8.10 (dd, J=8.9, 2.6, 1H), 7.86 (d, J=8.9, 1H), 7.70-7.65 (m, 1H), 7.46 (t, J=7.9, 1H), 7.42-7.34 (m, 3H), 7.30-7.26 (m, 1H), 7.12-7.07 (m, 1H), 7.04 (d, J=9.0, 2H), 6.49-6.44 (m, 1H), 4.53 (s, 2H), 4.26-4.00 (m, 4H), 3.54 (s, 4H), 1.98 (s, 4H).

### Example 46

### 7-[3-(4-Chloro-phenoxy)-benzyl]-2,7-diaza-spiro[3.5]nonane-2-carboxylic acid (6-[1,2,4]triazol-1-yl-pyridin-3-yl)-amide, trifluoroacetic acid salt

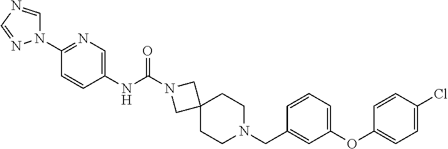

MS (ESI⁺): calcd for C₂₈H₂₈ClN₇O₂ m/z 529.20; found 530.2 (M+H)⁺. ¹H NMR (d₆-acetone): 9.11 (s, 1H), 8.62 (d, J=2.2, 1H), 8.19 (dd, J=8.9, 2.6, 1H), 8.10 (s, 1H), 7.78 (d, J=8.9, 1H), 7.46 (t, J=7.9, 1H), 7.42-7.35 (m, 3H), 7.30-7.27 (m, 1H), 7.12-7.07 (m, 1H), 7.05 (d, J=9.0, 2H), 4.54 (s, 2H), 4.13 (s, 4H), 3.56 (s, 4H), 1.99 (s, 4H).

### Example 47

### 7-[3-(4-Chloro-phenoxy)-benzyl]-2,7-diaza-spiro[3.5]nonane-2-carboxylic acid (6-[1,2,4]triazol-4-yl-pyridin-3-yl)-amide, trifluoroacetic acid salt

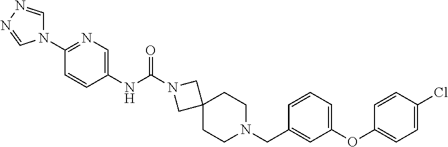

MS (ESI⁺): calcd for C₂₈H₂₈ClN₇O₂ m/z 529.20; found 530.2 (M+H)⁺. ¹H NMR (d₆-acetone): 9.05 (s, 2H), 8.65-8.62 (m, 1H), 8.46 (s, 1H), 8.26-8.21 (m, 1H), 7.72 (d, J=8.9, 1H), 7.46 (t, J=7.9, 1H), 7.42-7.36 (m, 3H), 7.30-7.28 (m, 1H), 7.12-7.08 (m, 1H), 7.05 (d, J=9.0, 2H), 4.52 (s, 2H), 4.21-4.01 (m, 4H), 3.56 (s, 4H), 2.03-1.91 (m, 4H).

### Example 48

### 7-[3-(4-Chloro-phenoxy)-benzyl]-2,7-diaza-spiro[3.5]nonane-2-carboxylic acid (6-chloro-pyridin-3-yl)-amide, trifluoroacetic acid salt

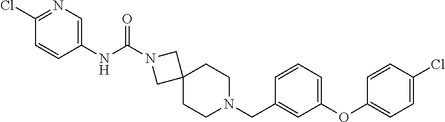

MS (ESI⁺): calcd for C₂₆H₂₆Cl₂N₄O₂ m/z 496.14; found 492.2 (M+H)⁺. ¹H NMR (d₆-acetone): 8.48 (d, J=2.7, 1H), 8.34 (s, 1H), 8.01 (dd, J=8.7, 2.8, 1H), 7.46 (t, J=7.9, 1H), 7.42-7.34 (m, 3H), 7.31-7.25 (m, 2H), 7.12-7.07 (m, 1H), 7.04 (d, J=8.9, 2H), 4.52 (s, 2H), 4.22-3.98 (m, 4H), 3.53 (s, 4H), 1.97 (s, 4H).

### Example 49

### 7-[3-(4-Chloro-phenoxy)-benzyl]-2,7-diaza-spiro[3.5]nonane-2-carboxylic acid (6-methoxy-pyridin-3-yl)-amide, trifluoroacetic acid salt

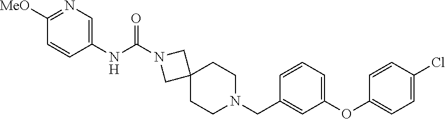

MS (ESI⁺): calcd for C₂₇H₂₉ClN₄O₃ m/z 492.19; found 493.2 (M+H)⁺. ¹H NMR (d₆-acetone): 8.24 (d, J=2.5, 1H), 7.87 (dd, J=8.9, 2.7, 1H), 7.47 (t, J=7.9, 1H), 7.42-7.34 (m, 3H), 7.29-7.26 (m, 1H), 7.13-7.08 (m, 1H), 7.05 (d, J=8.9, 2H), 6.75 (d, J=8.9, 1H), 4.56 (s, 2H), 4.25-4.07 (m, 4H), 3.86 (s, 3H), 3.51 (s, 4H), 1.96 (s, 4H).

### Example 50

### 7-[3-(4-Chloro-phenoxy)-benzyl]-2,7-diaza-spiro[3.5]nonane-2-carboxylic acid (6-cyano-pyridin-3-yl)-amide, trifluoroacetic acid salt

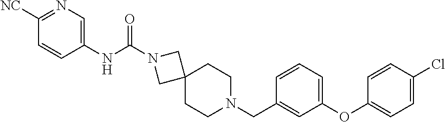

MS (ESI⁺): calcd for C₂₇H₂₆ClN₅O₂ m/z 487.18; found 488.2 (M+H)⁺. ¹H NMR (d₆-acetone): 8.78 (d, J=2.5, 1H), 8.69 (s, 1H), 8.20 (dd, J=8.6, 2.5, 1H), 7.74 (d, J=8.6, 1H), 7.46 (t, J=7.9, 1H), 7.42-7.35 (m, 3H), 7.30-7.26 (m, 1H), 7.09 (dd, J=8.1, 2.4, 1H), 7.04 (d, J=8.8, 2H), 4.52 (s, 2H), 4.33-4.12 (m, 4H), 3.56 (s, 4H), 1.99 (s, 4H).

### Example 51

### 7-[3-(4-Chloro-phenoxy)-benzyl]-2,7-diaza-spiro[3.5]nonane-2-carboxylic acid (1H-tetrazol-5-yl)-amide, trifluoroacetic acid salt

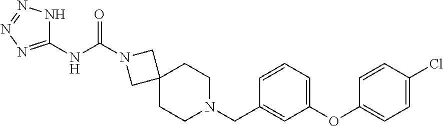

MS (ESI⁺): calcd for C₂₂H₂₄ClN₇O₂ m/z 453.17; found 454.2 (M+H)⁺. ¹H NMR (d₆-acetone): 7.45 (t, J=7.9, 1H), 7.41-7.36 (m, 3H), 7.30-7.27 (m, 1H), 7.11-7.07 (m, 1H), 7.05 (d, J=9.0, 2H), 4.50 (s, 2H), 4.09 (s, 4H), 3.67-3.61 (m, 4H), 2.04-2.00 (m, 4H).

### Example 52

### 7-[3-(4-Chloro-phenoxy)-benzyl]-2,7-diaza-spiro[3.5]nonane-2-carboxylic acid benzo[1,2,5]oxadiazol-4-ylamide, trifluoroacetic acid salt

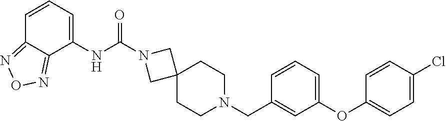

MS (ESI⁺): calcd for C₂₇H₂₆ClN₆O₃ m/z 503.17; found 504.2 (M+H)⁺. ¹H NMR (d₆-acetone): 8.29 (s, 1H), 7.93-7.86 (m, 1H), 7.53-7.44 (m, 3H), 7.42-7.36 (m, 3H), 7.32-7.28 (m, 1H), 7.12-7.08 (m, 1H), 7.05 (d, J=9.0, 2H), 4.52 (s, 2H), 4.22-4.01 (m, 4H), 3.62 (s, 4H), 2.03-1.93 (m, 4H).

### Example 53

### 7-[3-(4-Chloro-phenoxy)-benzyl]-2,7-diaza-spiro[3.5]nonane-2-carboxylic acid (4-chloro-pyridin-3-yl)-amide, trifluoroacetic acid salt

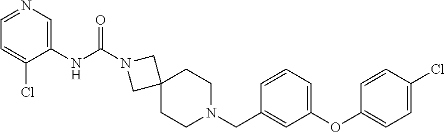

MS (ESI⁺): calcd for C₂₆H₂₆Cl₂N₄O₂ m/z 496.14; found 497.2 (M+H)⁺. ¹H NMR (d₆-acetone): 8.53 (s, 1H), 8.49 (s, 1H), 8.30 (d, J=5.3, 1H), 7.56 (d, J=5.3, 1H), 7.50-7.44 (m, 2H), 7.29 (d, J=7.7, 1H), 7.22-7.20 (m, 1H), 7.12 (dd, J=8.2, 1.7, 1H), 7.07 (d, J=9.0, 2H), 4.41 (d, J=5.6, 2H), 4.01-3.84 (m, 4H), 3.49-3.31 (m, 4H), 1.86-1.74 (m, 4H).

### Example 54

### 7-[3-(4-Chloro-phenoxy)-benzyl]-2,7-diaza-spiro[3.5]nonane-2-carboxylic acid (2-chloro-pyridin-3-yl)-amide, trifluoroacetic acid salt

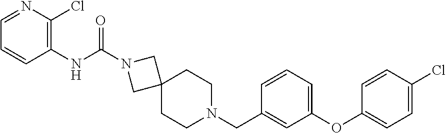

MS (ESI⁺): calcd for C₂₆H₂₆Cl₂N₄O₂ m/z 496.14; found 497.2 (M+H)⁺. ¹H NMR (d₆-DMSO): 8.53 (s, 1H), 8.49 (s, 1H), 8.30 (d, J=5.3, 1H), 7.56 (d, J=5.3, 1H), 7.50-7.44 (m, 2H), 7.29 (d, J=7.7, 1H), 7.23-7.19 (m, 1H), 7.12 (dd, J=8.2, 1.7, 1H), 7.07 (d, J=9.0, 2H), 4.41 (d, J=5.6, 2H), 4.00-3.85 (m, 4H), 3.48-3.32 (m, 4H), 1.85-1.77 (m, 4H).

### Example 55

### 7-[3-(4-Chloro-phenoxy)-benzyl]-2,7-diaza-spiro[3.5]nonane-2-carboxylic acid (6-morpholin-4-yl-pyridin-3-yl)-amide, trifluoroacetic acid salt

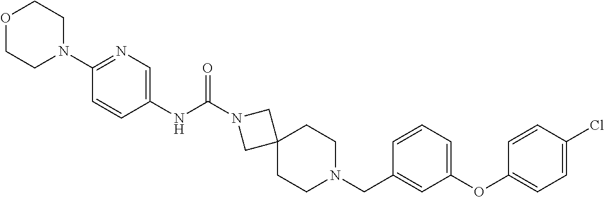

MS (ESI⁺): calcd for C₃₀H₃₄ClN₆O₃ m/z 547.24; found 548.3 (M+H)⁺. ¹H NMR (d₆-acetone): 8.52 (s, 1H), 8.18 (d, J=9.7, 1H), 7.46 (t, J=7.9, 1H), 7.42-7.34 (m, 4H), 7.28 (s, 1H), 7.10 (d, J=8.1, 1H), 7.04 (d, J=8.8, 2H), 4.54 (s, 2H), 4.22-4.05 (m, 4H), 3.86-3.79 (m, 4H), 3.74-3.65 (m, 4H), 3.52 (s, 4H), 2.03-1.89 (m, 4H).

### Example 56

### 7-[3-(4-Chloro-phenoxy)-benzyl]-2,7-diaza-spiro[3.5]nonane-2-carboxylic acid (1H-pyrazol-3-yl)-amide, trifluoroacetic acid salt

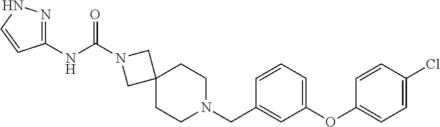

MS (ESI⁺): calcd for C₂₄H₂₆ClN₆O₂ m/z 451.18; found 548.3 (M+H)⁺. ¹H NMR (d₆-acetone): 7.88 (s, 1H), 7.46 (t, J=7.9, 1H), 7.41-7.34 (m, 3H), 7.30-7.26 (m, 1H), 7.10 (dd, J=8.2, 1.5, 1H), 7.04 (d, J=9.0, 2H), 6.49 (s, 1H), 4.57 (s, 2H), 4.17 (s, 4H), 3.56 (s, 4H), 1.98 (s, 4H).

### Example 57

### 7-[3-(4-Chloro-phenoxy)-benzyl]-2,7-diaza-spiro[3.5]nonane-2-carboxylic acid (5-chloro-pyridin-3-yl)-amide, trifluoroacetic acid salt

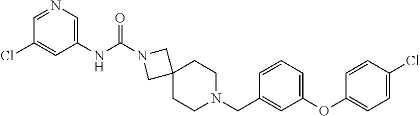

MS (ESI⁺): calcd for C₂₆H₂₆Cl₂N₄O₂ m/z 496.14; found 497.2 (M+H)⁺. ¹H NMR (d₆-acetone): 8.65 (d, J=2.1, 1H), 8.57 (5, 1H), 8.27-8.18 (m, 2H), 7.46 (t, J=7.9, 1H), 7.42-7.34 (m, 3H), 7.30-7.26 (m, 1H), 7.10 (dd, J=8.2, 1.5, 1H), 7.04 (d, J=9.0, 2H), 4.55 (5, 2H), 4.24-4.03 (m, 4H), 3.61-3.49 (m, 4H), 2.02-1.91 (m, 4H).

### Example 58

### 7-[3-(4-Chloro-phenoxy)-benzyl]-2,7-diaza-spiro[3.5]nonane-2-carboxylic acid (6-fluoro-pyridin-3-yl)-amide, trifluoroacetic acid salt

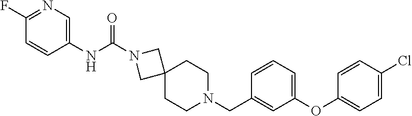

MS (ESI⁺): calcd for C₂₆H₂₆ClFN₄O₂ m/z 480.17; found 481.2 (M+H)⁺. ¹H NMR (d₆-acetone): 8.27 (s, 2H), 8.08 (t, J=6.6, 1H), 7.46 (t, J=7.9, 1H), 7.42-7.35 (m, 3H), 7.30-7.26 (m, 1H), 7.09 (dd, J=8.2, 1.5, 1H), 7.04 (d, J=9.0, 2H), 6.93 (dd, J=8.8, 3.3, 1H), 4.51 (s, 2H), 4.22-4.01 (m, 4H), 3.59-3.48 (m, 4H), 2.02-1.87 (m, 4H).

### Example 59

### 7-[3-(4-Chloro-phenoxy)-benzyl]-2,7-diaza-spiro[3.5]nonane-2-carboxylic acid (6-methoxy-pyrimidin-4-yl)-amide, trifluoroacetic acid salt

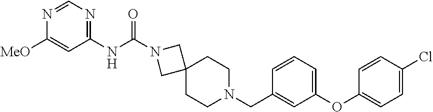

MS (ESI⁺): calcd for C₂₆H₂₈ClN₆O₃ m/z 493.19; found 494.2 (M+H)⁺. ¹H NMR (d₆-acetone): 8.43 (s, 1H), 7.46 (t, J=7.9, 1H), 7.41-7.36 (m, 3H), 7.33 (s, 1H), 7.30-7.28 (m, 1H), 7.10 (dd, J=8.2, 1.5, 1H), 7.05 (d, J=9.0, 2H), 4.55 (s, 2H), 4.24-4.07 (m, 4H), 3.94 (s, 3H), 3.63-3.55 (m, 4H), 2.04-1.96 (m, 4H).

### Example 60

### 7-[3-(4-Chloro-phenoxy)-benzyl]-2,7-diaza-spiro[3.5]nonane-2-carboxylic acid (6-chloro-pyridazin-3-yl)-amide, trifluoroacetic acid salt

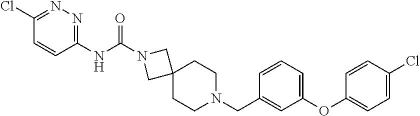

MS (ESI⁺): calcd for C₂₅H₂₅Cl₂N₅O₂ m/z 497.14; found 498.2 (M+H)⁺. ¹H NMR (d₆-acetone): 8.23 (d, J=9.4, 1H), 7.63 (d, J=9.4, 1H), 7.46 (t, J=7.9, 1H), 7.42-7.36 (m, 3H), 7.31-7.28 (m, 1H), 7.10 (dd, J=8.1, 1.5, 1H), 7.05 (d, J=9.0, 2H), 4.53 (s, 2H), 4.24-4.01 (m, 4H), 3.69-3.56 (m, 4H), 2.06-1.96 (m, 4H).

### Example 61

### 7-[3-(4-Chloro-phenoxy)-benzyl]-2,7-diaza-spiro[3.5]nonane-2-carboxylic acid (1,5-dimethyl-1H-pyrazol-3-yl)-amide, trifluoroacetic acid salt

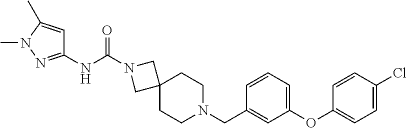

MS (ESI⁺): calcd for C₂₆H₃₀ClN₅O₂ m/z 479.21; found 480.2 (M+H)⁺. ¹H NMR (d₆-acetone): 7.46 (t, J=7.9, 1H), 7.41-7.36 (m, 3H), 7.31-7.27 (m, 1H), 7.10 (dd, J=8.1, 1.5, 1H), 7.04 (d, J=9.0, 2H), 6.46 (s, 1H), 4.55 (s, 2H), 4.23-4.00 (m, 4H), 3.79 (s, 3H), 3.58-3.47 (m, 4H), 2.34 (s, 3H), 2.01-1.90 (m, 4H).

### Example 62

### 7-[3-(4-Chloro-phenoxy)-benzyl]-2,7-diaza-spiro[3.5]nonane-2-carboxylic acid (4-bromo-1-methyl-1H-pyrazol-3-yl)-amide, trifluoroacetic acid salt

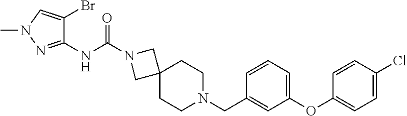

MS (ESI⁺): calcd for C₂₅H₂₇BrClN₅O₂ m/z 543.10; found 544.1 (M+H)⁺. ¹H NMR (d₆-acetone): 7.63 (s, 1H), 7.46 (t, J=7.9, 1H), 7.41-7.33 (m, 3H), 7.30-7.25 (m, 1H), 7.09 (d, J=7.9, 1H), 7.05 (d, J=8.9, 2H), 4.52 (s, 2H), 4.22-3.98 (m, 4H), 3.78 (s, 3H), 3.56-3.43 (m, 4H), 2.01-1.88 (m, 4H).

### Example 63

### 7-[3-(4-Chloro-phenoxy)-benzyl]-2,7-diaza-spiro[3.5]nonane-2-carboxylic acid (2-ethyl-2H-pyrazol-3-yl)-amide, trifluoroacetic acid salt

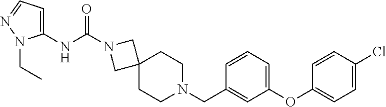

MS (ESI⁺): calcd for C₂₆H₃₀ClN₅O₂ m/z 479.21; found 480.2 (M+H)⁺. ¹H NMR (d₆-acetone): 7.47 (t, J=7.9, 1H), 7.42-7.37 (m, 3H), 7.36-7.35 (m, 1H), 7.29-7.27 (m, 1H), 7.10 (dd, J=8.2, 1.6, 1H), 7.05 (d, J=9.0, 2H), 6.03 (d, J=2.0, 1H), 4.53 (s, 2H), 4.20-4.07 (m, 4H), 4.02 (q, J=7.2, 7.2, 2H), 3.58-3.45 (m, 4H), 2.02-1.89 (m, 4H), 1.33 (t, J=7.2, 3H).

### Example 64

### 7-[3-(4-Chloro-phenoxy)-benzyl]-2,7-diaza-spiro[3.5]nonane-2-carboxylic acid (2H-tetrazol-5-yl)-amide, trifluoroacetic acid salt

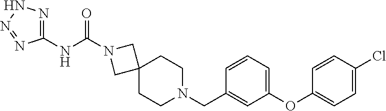

MS (ESI⁺): calcd for C₂₂H₂₄ClN₇O₂ m/z 453.17; found 454.2 (M+H)⁺. ¹H NMR (d₆-acetone): 7.45 (t, J=7.9, 1H), 7.41-7.36 (m, 3H), 7.30-7.28 (m, 1H), 7.09 (dd, J=8.1, 1.4, 1H), 7.05 (d, J=9.0, 2H), 4.49 (s, 2H), 4.16-4.01 (m, 4H), 3.67-3.61 (m, 4H), 2.04-1.99 (m, 4H).

### Example 65

### 7-[3-(4-Chloro-phenoxy)-benzyl]-2,7-diaza-spiro[3.5]nonane-2-carboxylic acid (2-methyl-benzooxazol-5-yl)-amide

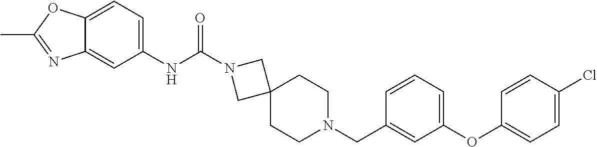

MS (ESI⁺): calcd for C₂₉H₂₉ClN₄O₃ m/z 516.19; found 517.2 (M+H)⁺. ¹H NMR (CDCl₃): 7.62 (d, J=2.0, 1H), 7.44 (d, J=8.8, 1H), 7.34 (d, J=6.9, 3H), 7.28 (dd, J=8.8, 2.1, 1H), 7.10 (d, J=7.6, 1H), 6.99-6.94 (m, 3H), 6.90 (dd, J=8.2, 1.6, 1H), 3.67 (s, 2H), 3.49-3.41 (m, 4H), 3.15 (s, 4H), 2.61 (s, 3H), 1.82-1.72 (m, 4H).

### Example 66

### 7-[3-(4-Chloro-phenoxy)-benzyl]-2,7-diaza-spiro[3.5]nonane-2-carboxylic acid isoxazolo[5,4-b]pyridin-3-ylamide, trifluoroacetic acid salt

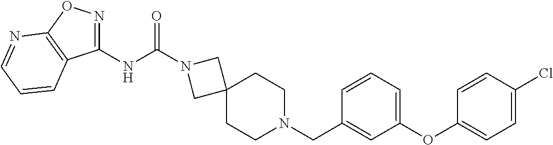

MS (ESI⁺): calcd for C₂₇H₂₆ClN₆O₃ m/z 503.17; found 504.2 (M+H)⁺. ¹H NMR (d₆-acetone): 8.22 (dd, J=7.0, 2.0, 1H), 7.72 (dd, J=6.2, 1.7, 1H), 7.45 (t, J=7.9, 1H), 7.37 (d, J=9.0, 3H), 7.28-7.26 (m, 1H), 7.09 (dd, J=8.1, 1.5, 1H), 7.02 (d, J=9.0, 2H), 6.45 (t, J=6.7, 1H), 4.55 (s, 2H), 4.27-4.07 (m, 4H), 3.69-3.61 (m, 4H), 2.14-2.07 (m, 4H).

### Example 67

### 7-[3-(4-Chloro-phenoxy)-benzyl]-2,7-diaza-spiro[3.5]nonane-2-carboxylic acid isoxazolo[4,5-b]pyridin-3-ylamide, trifluoroacetic acid salt

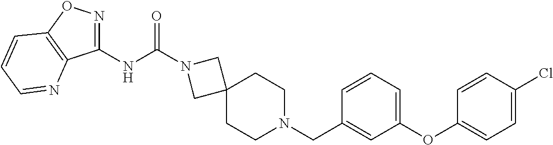

MS (ESI⁺): calcd for C₂₇H₂₆ClN₅O₃ m/z 503.17; found 504.2 (M+H)⁺. ¹H NMR (d₆-acetone): 8.67 (dd, J=4.4, 0.8, 1H), 8.04 (dd, J=8.6, 1.1, 1H), 7.65 (dd, J=8.6, 4.5, 1H), 7.45 (t, J=7.9, 1H), 7.40-7.34 (m, 3H), 7.29-7.25 (m, 1H), 7.09 (dd, J=8.1, 1.5, 1H), 7.02 (d, J=9.0, 2H), 4.57 (s, 2H), 4.30-4.09 (m, 4H), 3.70-3.60 (m, 4H), 2.05-2.01 (m, 4H).

### Example 68

### 7-[3-(4-Chloro-phenoxy)-benzyl]-2,7-diaza-spiro[3.5]nonane-2-carboxylic acid (1H-indazol-7-yl)-amide, trifluoroacetic acid salt

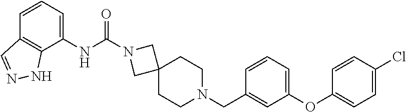

MS (ESI⁺): calcd for C₂₈H₂₈ClN₅O₂ m/z 501.19; found 502.2 (M+H)⁺. ¹H NMR (d₆-acetone): 8.00 (s, 1H), 7.48-7.43 (m, 2H), 7.39 (d, J=9.0, 2H), 7.35 (d, J=7.7, 1H), 7.28-7.26 (m, 1H), 7.24 (d, J=6.9, 1H), 7.09 (dd, J=8.2, 1.5, 1H), 7.04 (d, J=9.0, 2H), 7.00 (d, J=7.6, 1H), 4.48 (s, 2H), 4.19-3.97 (m, 4H), 3.64-3.49 (m, 4H), 2.03-1.86 (m, 4H).

### Example 69

### 7-[3-(4-Chloro-phenoxy)-benzyl]-2,7-diaza-spiro[3.5]nonane-2-carboxylic acid imidazo[1,2-a]pyridin-6-ylamide, trifluoroacetic acid salt

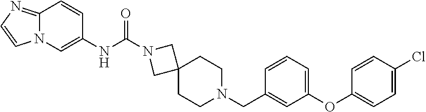

MS (ESI⁺): calcd for C₂₈H₂₈ClN₅O₂ m/z 501.19; found 502.2 (M+H)⁺. ¹H NMR (d₆-acetone): 9.40 (5, 1H), 8.29 (5, 1H), 8.03 (d, J=5.0, 2H), 7.95 (d, J=9.0, 1H), 7.46 (t, J=7.9, 1H), 7.41-7.34 (m, 3H), 7.30-7.26 (m, 1H), 7.09 (dd, J=8.1, 1.6, 1H), 7.04 (d, J=9.0, 2H), 4.52 (5, 2H), 4.22-4.01 (m, 4H), 3.63-3.51 (m, 4H), 2.03-1.90 (m, 4H).

### Example 70

### 7-[3-(4-Chloro-phenoxy)-benzyl]-2,7-diaza-spiro[3.5]nonane-2-carboxylic acid (6-methoxy-pyridazin-3-yl)-amide, trifluoroacetic acid salt

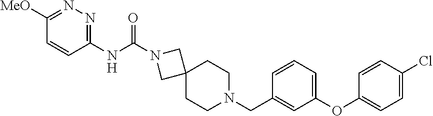

MS (ESI⁺): calcd for C₂₆H₂₈ClN₅O₃ m/z 493.19; found 494.2 (M+H)⁺. ¹H NMR (d₆-acetone): 8.48 (s, 1H), 7.71 (d, J=8.6, 1H), 7.47 (t, J=7.9, 1H), 7.42-7.35 (m, 3H), 7.31-7.27 (m, 1H), 7.10 (dd, J=8.2, 1.5, 1H), 7.04 (d, J=9.0, 2H), 4.57 (s, 2H), 4.29-4.09 (m, 4H), 4.03 (s, 3H), 3.68-3.59 (m, 4H), 2.06-1.99 (m, 4H).

### Example 71

### 7-[3-(4-Chloro-phenoxy)-benzyl]-2,7-diaza-spiro[3.5]nonane-2-carboxylic acid (2-trifluoromethyl-pyrimidin-4-yl)-amide, trifluoroacetic acid salt

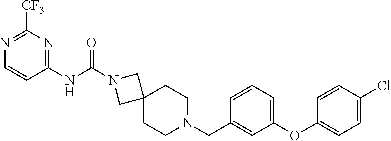

MS (ESI⁺): calcd for C₂₆H₂₅ClF₃N₅O₂ m/z 531.16; found 532.2 (M+H)⁺. ¹H NMR (d₆-acetone): 9.14 (s, 1H), 8.65 (d, J=5.9, 1H), 8.12 (d, J=5.9, 1H), 7.46 (t, J=7.9, 1H), 7.42-7.36 (m, 3H), 7.32-7.27 (m, 1H), 7.09 (dd, J=8.1, 2.2, 1H), 7.05 (d, J=8.9, 2H), 4.52 (s, 2H), 4.24-4.07 (m, 4H), 3.69-3.58 (m, 4H), 2.04-1.96 (m, 4H).

### Example 72

### 7-[3-(4-Chloro-phenoxy)-benzyl]-2,7-diaza-spiro[3.5]nonane-2-carboxylic acid (2-methoxy-pyrimidin-4-yl)-amide, trifluoroacetic acid salt

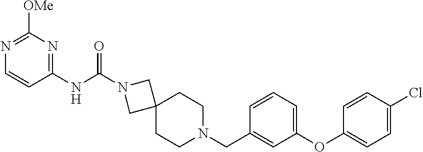

MS (ESI⁺): calcd for C₂₆H₂₈ClN₅O₃ m/z 493.19; found 494.2 (M+H)⁺. ¹H NMR (d₆-acetone): 8.93-8.84 (m, 1H), 7.70 (s, 1H), 7.47 (t, J=7.9, 1H), 7.42-7.35 (m, 3H), 7.30-7.27 (m, 1H), 7.10 (dd, J=8.2, 1.5, 1H), 7.04 (d, J=9.0, 2H), 4.57 (s, 2H), 4.29-4.07 (m, 4H), 3.97 (s, 3H), 3.67-3.55 (m, 4H), 2.04-1.96 (m, 4H).

### Example 73

### 7-[3-(4-Chloro-phenoxy)-benzyl]-2,7-diaza-spiro[3.5]nonane-2-carboxylic acid (5-fluoro-pyridin-3-yl)-amide, trifluoroacetic acid salt

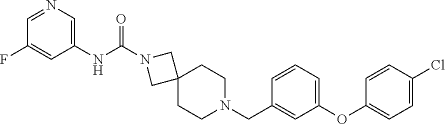

MS (ESI⁺): calcd for C₂₆H₂₆ClFN₄O₂ m/z 480.17; found 481.2 (M+H)⁺. ¹H NMR (d₆-acetone): 8.69 (s, 1H), 8.17-8.02 (m, 2H), 7.47 (t, J=7.9, 1H), 7.41-7.35 (m, 3H), 7.30-7.26 (m, 1H), 7.10 (dd, J=8.2, 1.6, 1H), 7.04 (d, J=9.0, 2H), 4.56 (s, 2H), 4.26-4.06 (m, 4H), 3.63-3.48 (m, 4H), 2.04-1.91 (m, 4H).

### Example 74

### 7-[3-(4-Chloro-phenoxy)-benzyl]-2,7-diaza-spiro[3.5]nonane-2-carboxylic acid (1H-pyrrolo[2,3-b]pyridin-4-yl)-amide, trifluoroacetic acid salt

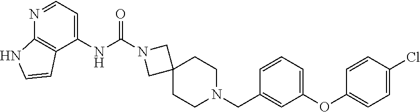

MS (ESI⁺): calcd for C₂₈H₂₈ClN₅O₂ m/z 501.19; found 502.2 (M+H)⁺. ¹H NMR (d₆-acetone): 8.02-7.98 (m, 2H), 7.65-7.60 (m, 1H), 7.46 (t, J=7.9, 1H), 7.42-7.37 (m, 3H), 7.31-7.27 (m, 1H), 7.27-7.24 (m, 1H), 7.11-7.03 (m, 3H), 4.50 (s, 2H), 4.20-4.04 (m, 4H), 3.76-3.66 (m, 4H), 3.35-3.27 (m, 4H), 2.33-2.24 (m, 2H).

### Example 75

### 7-(3-(4-chlorophenoxy)benzyl)-N-(1,3-dimethyl-1H-pyrazolo[3,4-b]pyridin-5-yl)-2,7-diazaspiro[3.5]nonane-2-carboxamide, trifluoroacetic acid salt.

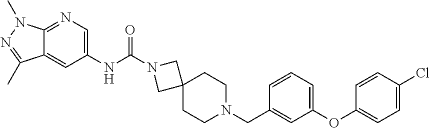

MS (ESI⁺): calcd for C₂₉H₃₁ClN₆O₂ m/z 530.22; found 531.2 (M+H)⁺. ¹H NMR (d₆-acetone): 8.50 (s, 1H), 8.18 (d, J=2.2, 1H), 7.46 (t, J=7.9, 1H), 7.42-7.35 (m, 3H), 7.30-7.27 (m, 1H), 7.10 (dd, J=8.2, 1.6, 1H), 7.05 (d, J=9.0, 2H), 4.55 (s, 2H), 4.25-4.05 (m, 4H), 3.94 (s, 3H), 3.61-3.49 (m, 4H), 2.43 (s, 3H), 2.03-1.92 (m, 4H).

### Example 76

### 7-(3-(4-chlorophenoxy)benzyl)-N-(5-methylisoxazol-3-yl)-2,7-diazaspiro[3.5]nonane-2-carboxamide, trifluoroacetic acid salt.

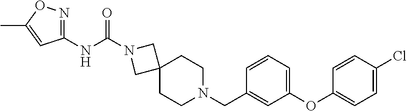

MS (ESI⁺): calcd for C₂₅H₂₇ClN₄O₃ m/z 466.18; found 467.2 (M+H)⁺. ¹H NMR (d₆-acetone): 8.73 (s, 1H), 7.45 (t, J=7.9, 1H), 7.41-7.35 (m, 3H), 7.30-7.27 (m, 1H), 7.09 (dd, J=8.2, 1.5, 1H), 7.04 (d, J=9.0, 2H), 6.53 (s, 1H), 4.51 (s, 2H), 4.25-3.97 (m, 4H), 3.60-3.47 (m, 4H), 2.33 (s, 3H), 2.02-1.90 (m, 4H).

### Example 77

### 7-(3-(4-chlorophenoxy)benzyl)-N-(2-methylbenzo[d]thiazol-6-yl)-2,7-diazaspiro[3.5]nonane-2-carboxamide, trifluoroacetic acid salt.

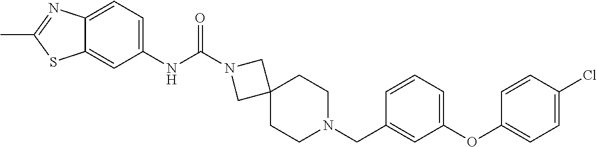

MS (ESI⁺): calcd for C₂₉H₂₉ClN₄O₂S m/z 532.17; found 533.2 (M+H)⁺. ¹H NMR (d₆-acetone): 8.22 (d, J=1.9, 1H), 7.72 (d, J=8.8, 1H), 7.49-7.43 (m, 2H), 7.41-7.35 (m, 3H), 7.29-7.27 (m, 1H), 7.09 (dd, J=8.2, 1.5, 1H), 7.04 (d, J=9.0, 2H), 4.52 (s, 2H), 4.21-4.01 (m, 4H), 3.58-3.46 (m, 4H), 2.74 (s, 3H), 2.01-1.88 (m, 4H).

### Example 78

### 7-(3-(4-chlorophenoxy)benzyl)-N-(5-methyl-1H-pyrazol-3-yl)-2,7-diazaspiro[3.5]nonane-2-carboxamide, trifluoroacetic acid salt.

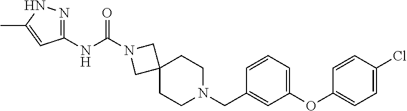

MS (ESI⁺): calcd for C₂₅H₂₈ClN₅O₂ m/z 465.19; found 466.2 (M+H)⁺. ¹H NMR (d₆-acetone): 7.46 (t, J=7.9, 1H), 7.42-7.36 (m, 3H), 7.32-7.26 (m, 2H), 7.12-7.07 (m, 1H), 7.04 (d, J=9.0, 2H), 4.54 (s, 2H), 4.24-4.03 (m, 4H), 3.63-3.46 (m, 4H), 2.36 (s, 3H), 2.01-1.91 (m, 4H).

### Example 79

### 7-(3-(4-chlorophenoxy)benzyl)-N-(5-methylpyridin-3-yl)-2,7-diazaspiro[3.5]nonane-2-carboxamide, trifluoroacetic acid salt.

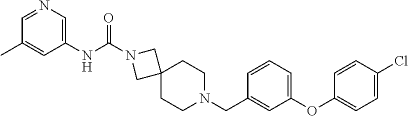

MS (ESI⁺): calcd for C₂₇H₂₉ClN₄O₂ m/z 476.20; found 477.2 (M+H)⁺. ¹H NMR (d₆-acetone): 9.42-9.27 (m, 1H), 9.16 (s, 1H), 8.40 (s, 1H), 7.45 (t, J=7.9, 1H), 7.41-7.34 (m, 3H), 7.29-7.26 (m, 1H), 7.08 (dd, J=8.1, 2.3, 1H), 7.03 (d, J=8.9, 2H), 4.53 (s, 2H), 4.24-4.05 (m, 4H), 3.63-3.52 (m, 4H), 2.50 (s, 3H), 2.03-1.92 (m, 4H).

### Example 80

### 7-(3-(4-chlorophenoxy)benzyl)-N-(2-fluoropyridin-3-yl)-2,7-diazaspiro[3.5]nonane-2-carboxamide, trifluoroacetic acid salt.

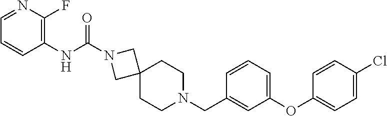

MS (ESI⁺): calcd for C₂₆H₂₆ClFN₄O₂ m/z 480.17; found 481.2 (M+H)⁺. ¹H NMR (d₆-acetone): 8.28 (t, J=9.0, 1H), 7.80 (d, J=4.8, 1H), 7.46 (t, J=7.9, 1H), 7.42-7.35 (m, 3H), 7.30-7.27 (m, 1H), 7.21 (dd, J=6.7, 4.8, 1H), 7.10 (dd, J=8.2, 1.5, 1H), 7.05 (d, J=9.0, 2H), 4.54 (s, 2H), 4.24-4.02 (m, 4H), 3.62-3.48 (m, 4H), 2.03-1.89 (m, 4H).

### Example 81

### 7-(3-(4-chlorophenoxy)benzyl)-N-(6-(piperidin-1-yl)pyridin-3-yl)-2,7-diazaspiro[3.5]nonane-2-carboxamide, trifluoroacetic acid salt.

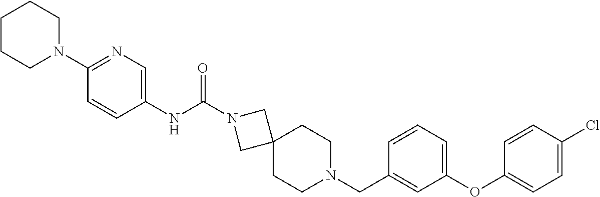

MS (ESI⁺): calcd for O₃₁H₃₆ClN₅O₂ m/z 545.26; found 546.3 (M+H)⁺. ¹H NMR (d₆-acetone): 8.45 (s, 1H), 8.12 (dd, J=9.8, 2.3, 1H), 7.45 (t, J=7.9, 1H), 7.41-7.34 (m, 3H), 7.32 (d, J=9.8, 1H), 7.29-7.26 (m, 1H), 7.08 (dd, J=8.2, 1.6, 1H), 7.04 (d, J=9.0, 2H), 4.51 (s, 2H), 4.20-4.00 (m, 4H), 3.75-3.65 (m, 4H), 3.56-3.44 (m, 4H), 2.00-1.87 (m, 4H), 1.72 (s, 6H).

### Example 82

### N-(5-bromopyridin-3-yl)-7-(3-(4-chlorophenoxy)benzyl)-2,7-diazaspiro[3.5]nonane-2-carboxamide, trifluoroacetic acid salt.

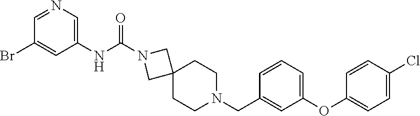

MS (ESI⁺): calcd for C₂₆H₂₆BrClN₄O₂ m/z 540.09; found 541.1 (M+H)⁺. ¹H NMR (d₆-acetone): 8.54 (s, 1H), 8.39 (s, 1H), 7.46 (t, J=7.9, 1H), 7.43-7.35 (m, 3H), 7.31-7.27 (m, 1H), 7.10 (dd, J=8.1, 2.4, 1H), 7.05 (d, J=9.0, 2H), 6.64 (s, 1H), 4.54 (s, 2H), 4.24-4.01 (m, 4H), 3.63-3.47 (m, 4H), 2.03-1.86 (m, 4H).

### Example 83

### 7-(3-(4-chlorophenoxy)benzyl)-N-(2-phenylpyrimidin-5-yl)-2,7-diazaspiro[3.5]nonane-2-carboxamide, trifluoroacetic acid salt.

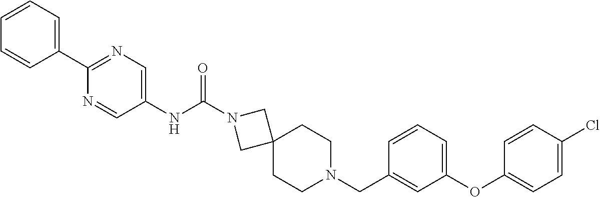

MS (ESI⁺): calcd for O₃₁H₃₀ClN₅O₂ m/z 539.21; found 540.2 (M+H)⁺. ¹H NMR (d₆-acetone): 9.02 (s, 2H), 8.50 (s, 1H), 8.44-8.37 (m, 2H), 7.53-7.35 (m, 7H), 7.31-7.27 (m, 1H), 7.10 (dd, J=8.2, 2.2, 1H), 7.05 (d, J=8.9, 2H), 4.56 (s, 2H), 4.28-4.06 (m, 4H), 3.66-3.49 (m, 4H), 2.04-1.89 (m, 4H).

### Example 84

### 7-(3-(4-chlorophenoxy)benzyl)-N-(4-cyanopyridin-3-yl)-2,7-diazaspiro[3.5]nonane-2-carboxamide.

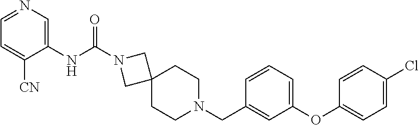

MS (ESI⁺): calcd for C₂₇H₂₆ClN₅O₂ m/z 487.18; found 488.2 (M+H)⁺. ¹H NMR (CDCl₃): 9.44 (s, 1H), 8.38 (d, J=5.0, 1H), 7.38 (dd, J=5.0, 0.7, 1H), 7.30-7.25 (m, 3H), 7.04 (d, J=7.9, 1H), 6.96-6.89 (m, 4H), 6.87 (dd, J=8.1, 1.6, 1H), 3.63 (s, 2H), 3.49-3.43 (m, 4H), 3.08 (s, 4H), 1.89-1.80 (m, 4H).

### Example 85

### 7-(3-(4-chlorophenoxy)benzyl)-N-(4-methylpyridin-3-yl)-2,7-diazaspiro[3.5]nonane-2-carboxamide.

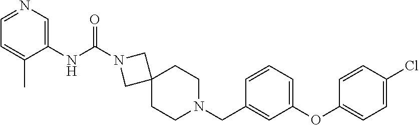

MS (ESI⁺): calcd for C₂₇H₂₉ClN₄O₂ m/z 476.20; found 477.2 (M+H)⁺. ¹H NMR (CDCl₃): 8.58 (s, 1H), 8.23 (d, J=4.9, 1H), 7.31-7.23 (m, 3H), 7.08 (d, J=4.9, 1H), 7.03 (d, J=7.6, 1H), 6.96-6.91 (m, 3H), 6.86 (dd, J=8.1, 1.6, 1H), 6.28 (s, 1H), 3.62 (s, 2H), 3.44-3.37 (m, 4H), 3.06 (s, 4H), 2.23 (s, 3H), 1.84-1.76 (m, 4H).

### Example 86

### 7-(3-(4-chlorophenoxy)benzyl)-N-(4-(trifluoromethyl)pyridin-3-yl)-2,7-diazaspiro[3.5]nonane-2-carboxamide.

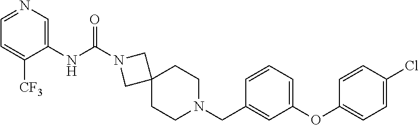

MS (ESI⁺): calcd for C₂₇H₂₆ClF₃N₄O₂ m/z 530.17; found 531.2 (M+H)⁺. ¹H NMR (CDCl₃): 9.34 (s, 1H), 8.44 (dd, J=5.1, 0.7, 1H), 7.41 (d, J=5.1, 1H), 7.30-7.24 (m, 3H), 7.03 (d, J=7.6, 1H), 6.96-6.90 (m, 3H), 6.86 (dd, J=8.1, 1.7, 1H), 6.69 (s, 1H), 3.62 (s, 2H), 3.45-3.39 (m, 4H), 3.07 (s, 4H), 1.86-1.79 (m, 4H).

### Example 87

### 7-(2,2-Difluoro-benzo[1,3]-dioxol-5-ylmethyl)-2,7-diaza-spiro[3.5]nonane-2-carboxylic acid pyridin-3-ylamide.

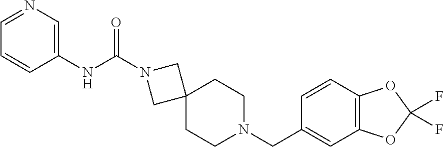

MS (ESI⁺): calcd for C₂₁H₂₂F₂N₄O₃ m/z 416.17; found 417.5 (M+H)⁺. ¹H NMR (CDCl₃): 8.44 (d, J=2.2, 1H), 8.25 (d, J=3.9, 1H), 8.07-8.03 (m, 1H), 7.22 (dd, J=8.3, 4.6, 1H), 7.09 (s, 1H), 6.97 (d, J=0.7, 2H), 6.28 (s, 1H), 3.76 (s, 4H), 3.43 (s, 2H), 2.34 (s, 4H), 1.80 (t, J=5.4, 4H).

### Example 88

### 7-(2,2-Difluoro-benzo[1,3]dioxol-5-ylmethyl)-2,7-diaza-spiro[3.5]nonane-2-carboxylic acid (6-[1,2,3]triazol-2-yl-pyridin-3-yl)-amide.

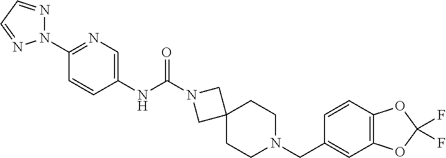

MS (ESI⁺): calcd for C₂₃H₂₃F₂N₇O₃ m/z 483.18; found 484.5 (M+H)⁺. ¹H NMR (CDCl₃): 8.39 (d, J=2.6, 1H), 8.33 (dd, J=8.9, 2.7, 1H), 7.99 (d, J=8.9, 1H), 7.85 (s, 2H), 7.08 (s, 1H), 6.97 (d, J=0.9, 2H), 6.48 (s, 1H), 3.78 (s, 4H), 3.42 (s, 2H), 2.34 (s, 4H), 1.80 (t, J=5.4, 4H).

### Example 89

### 7-(2,2-Difluoro-benzo[1,3]dioxol-5-ylmethyl)-2,7-diaza-spiro[3.5]nonane-2-carboxylic acid (1H-pyrrolo[2,3-b]pyridin-5-yl)-amide.

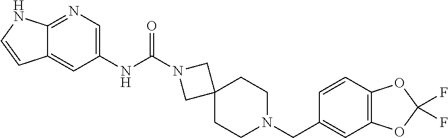

MS (ESI⁺): calcd for C₂₃H₂₃F₂N₅O₃ m/z 455.18; found 456.5 (M+H)⁺. ¹H NMR (CDCl₃): 9.50 (s, 1H), 8.20 (d, J=2.3, 1H), 8.09 (d, J=2.3, 1H), 7.29 (d, J=3.5, 1H), 7.09 (s, 1H), 6.97 (d, J=0.8, 2H), 6.44 (d, J=3.5, 1H), 6.18 (s, 1H), 3.74 (s, 4H), 3.42 (s, 2H), 2.34 (s, 4H), 1.79 (t, J=5.41, 4H).

### Example 90

### 8-[3-(4-Chloro-phenoxy)-benzyl]-2,8-diaza-spiro[4.5]decane-2-carboxylic acid pyridin-3-ylamide, trifluoroacetic acid salt

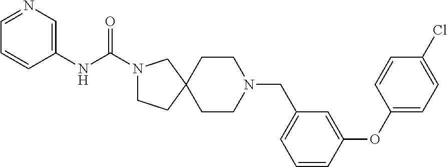

Step A: 2-(Pyridin-3-ylcarbamoyl)-2,8-diaza-spiro[4.5]decane-8-carboxylic acid tert-butyl ester. To a solution of 2,8-diaza-spiro[4.5]decane-8-carboxylic acid tert-butyl ester hydrochloride (0.100 g, 0.36 mmol) and TEA (0.148 mL, 1.083 mmol) in MeCN (3 mL) was added pyridin-3-yl-carbamic acid phenyl ester (0.085 g, 0.40 mmol). The reaction mixture was heated at 50° C. overnight, then diluted with EtOAc (15 mL) and washed with saturated aq. NaHCO₃ (15 mL). The organic layer was dried (Na₂SO₄) and concentrated to dryness. The crude residue was purified (FCC) to give 2-(pyridin-3-ylcarbamoyl)-2,8-diaza-spiro[4.5]decane-8-carboxylic acid tert-butyl ester as a yellow oil (0.097 g, 75%).

Step B: 2,8-Diaza-spiro[4.5]decane-2-carboxylic acid pyridin-3-ylamide hydrochloride. To a solution of 2-(pyridin-3-ylcarbamoyl)-2,8-diaza-spiro[4.5]decane-8-carboxylic acid tert-butyl ester (0.097 g, 0.27 mmol) in CH₂Cl₂ (5 mL) was treated with 4 M HCl/dioxane (0.451 mL, 1.81 mmol) and stirred overnight. The resulting white precipitate was filtered and dried under vacuum to give 2,8-diaza-spiro[4.5]decane-2-carboxylic acid pyridin-3-ylamide hydrochloride as a white solid (0.079 g, 99%).

Step C: 8-[3-(4-Chloro-phenoxy)-benzyl]-2,8-diaza-spiro[4.5]decane-2-carboxylic acid pyridin-3-ylamide, trifluoroacetic acid salt. To a suspension of 2,8-diaza-spiro[4.5]decane-2-carboxylic acid pyridin-3-ylamide hydrochloride (0.079 g, 0.266 mmol) in THF (5 mL) were added TEA (0.049 mL, 0.361 mmol) and 3-(4-chloro-phenoxy)-benzaldehyde (0.076 mL, 0.397 mmol). After 15 min of stirring, the reaction mixture was treated with NaB(OAc)₃H (0.191 g, 0.903 mmol) and stirred overnight. The reaction was quenched with saturated aq. NaHCO₃ (30 mL). The aqueous phase was extracted with EtOAc (2×30 mL). The organic layers were combined and washed with saturated aq. NaCl (2×50 mL). The organic layer was isolated, dried over Na₂SO₄, filtered and concentrated. The crude residue was purified via HPLC to give 8-[3-(4-chloro-phenoxy)-benzyl]-2,8-diaza-spiro[4.5]decane-2-carboxylic acid pyridin-3-ylamide, trifluoroacetic acid salt as a white solid (0.040 g, 19%).

MS (ESI⁺): calcd for C₂₇H₂₉ClN₄O₂ m/z 476.20; found 477.2 (M+H)⁺. ¹H NMR (d₆-DMSO): 10.38 (s, 1H), 8.72-8.63 (m, 1H), 8.13 (d, J=7.7, 1H), 7.53-7.43 (m, 4H), 7.39-7.34 (m, 1H), 7.27 (s, 1H), 7.14-7.07 (m, 3H), 4.31 (s, 2H), 3.56-3.47 (m, 4H), 3.12-2.94 (m, 4H), 1.95-1.72 (m, 6H).

Examples 91 to 131 were prepared using methods analogous to those described for Example 90, using the appropriate carbamate, BOC-diazaspirocycle, and aldehyde.

### Example 91

### 8-[3-(4-Chloro-phenoxy)-benzyl]-2,8-diaza-spiro[4.5]decane-2-carboxylic acid (6-[1,2,3]triazol-2-yl-pyridin-3-yl)-amide, trifluoroacetic acid salt

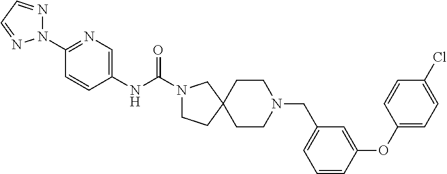

MS (ESI⁺): calcd for C₂₉H₃₀ClN₇O₂ m/z 543.22; found 544.2 (M+H)⁺. ¹H NMR (d₆-DMSO): 8.76-8.63 (m, 2H), 8.23 (d, J=8.8, 1H), 8.11 (s, 2H), 7.90 (s, 1H), 7.45 (d, J=7.9, 3H), 7.16-7.01 (m, 4H), 4.31 (s, 1H), 3.58-3.41 (m, 4H), 3.31-3.22 (m, 4H), 1.88-1.72 (m, 4H), 1.65-1.43 (m, 3H).

### Example 92

### 8-[3-(4-Chloro-phenoxy)-benzyl]-2,8-diaza-spiro[4.5]decane-2-carboxylic acid benzo[d]isoxazol-3-ylamide, trifluoroacetic acid salt

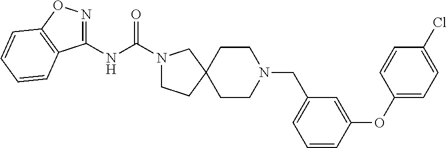

MS (ESI⁺): calcd for C₂₉H₂₉ClN₄O₃ m/z 516.19; found 517.2 (M+H)⁺. ¹H NMR (d₆-DMSO): 9.54 (s, 1H), 7.91 (d, J=7.9, 1H), 7.65-7.57 (m, 2H), 7.52-7.42 (m, 3H), 7.37-7.23 (m, 2H), 7.16-7.02 (m, 3H), 4.44-4.19 (m, 2H), 3.73-3.39 (m, 4H), 3.33-3.22 (m, 4H), 3.15-2.91 (m, 2H), 1.91-1.67 (m, 4H).

### Example 93

### 1-[3-(4-Chloro-phenoxy)-benzyl]-1,8-diaza-spiro[4.5]decane-8-carboxylic acid pyridin-3-ylamide

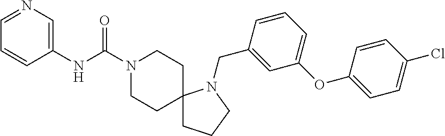

MS (ESI⁺): calcd for C₂₇H₂₉ClN₄O₂ m/z 476.20; found 477.5 (M+H)⁺. ¹H NMR (CDCl₃): 8.43 (d, J=2.5, 1H), 8.23 (dd, J=4.7, 1.4, 1H), 7.98-7.94 (m, 1H), 7.28-7.24 (m, 2H), 7.20 (dd, J=8.4, 4.7, 1H), 7.06 (d, J=7.7, 1H), 7.01-6.98 (m, 1H), 6.95-6.89 (m, 3H), 6.82 (dd, J=8.1, 3.2, 1H), 4.13 (d, J=13.5, 2H), 3.57 (s, 2H), 2.95 (t, J=14.2, 2H), 2.68 (t, J=6.6, 2H), 1.85-1.65 (m, 6H), 1.45 (d, J=12.4, 2H).

### Example 94

### 1-[3-(4-Chloro-phenoxy)-benzyl]-1,8-diaza-spiro[4.5]decane-8-carboxylic acid (6-[1,2,3]triazol-2-yl-pyridin-3-yl)-amide

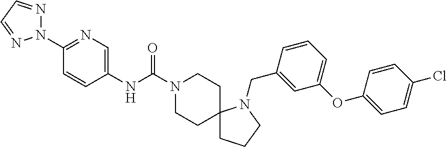

MS (ESI⁺): calcd for C₂₉H₃₀ClN₇O₂ m/z 543.22; found 544.6 (M+H)⁺. ¹H NMR (CDCl₃): 8.38 (d, J=2.6, 1H), 8.26 (dd, J=9.0, 2.7, 1H), 7.98 (d, J=8.9, 1H), 7.83 (s, 2H), 7.29-7.22 (m, 3H), 7.05 (d, J=7.6, 1H), 7.03-6.98 (m, 2H), 6.91 (d, J=9.0, 2H), 6.82 (dd, J=8.1, 1.6, 1H), 4.20-4.11 (m, 2H), 3.57 (s, 2H), 2.97 (t, J=12.0, 2H), 2.67 (t, J=6.4, 2H), 1.85-1.68 (s, 6H), 1.46 (d, J=12.5, 2H).

### Example 95

### 1-[3-(4-Chloro-phenoxy)-benzyl]-1,8-diaza-spiro[4.5]decane-8-carboxylic acid (1H-pyrrolo[2,3-b]pyridin-5-yl)-amide

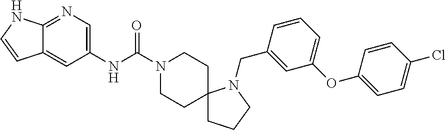

MS (ESI⁺): calcd for C₂₉H₃₀ClN₅O₂ m/z 515.21; found 516.6 (M+H)⁺. ¹H NMR (CDCl₃): 9.71 (s, 1H), 8.17 (d, J=2.2, 1H), 8.04 (d, J=2.2, 1H), 7.33-7.22 (m, 4H), 7.07 (d, J=7.5, 1H), 7.03-6.99 (m, 1H), 6.92 (d, J=8.9, 2H), 6.83 (dd, J=8.1, 2.4, 1H), 6.55 (s, 1H), 6.45-6.40 (m, 1H), 4.17-4.08 (m, 2H), 3.58 (s, 2H), 2.96 (t, J=12.1, 2H), 2.68 (t, J=6.5, 2H), 1.87-1.69 (m, 6H), 1.45 (d, J=12.7, 2H).

### Example 96

### 6-[3-(4-Chloro-phenoxy)-benzyl]-2,6-diaza-spiro[3.5]nonane-2-carboxylic acid pyridin-3-ylamide

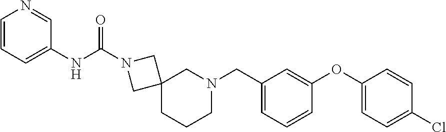

MS (ESI⁺): calcd for C₂₆H₂₇ClN₄O₂ m/z 462.18; found 463.2 (M+H)⁺. ¹H NMR (CDCl₃): 8.43 (d, J=2.6, 1H), 8.24 (dd, J=4.7, 1.4, 1H), 8.06-8.03 (m, 1H), 7.31-7.25 (m, 3H), 7.21 (dd, J=8.4, 4.7, 1H), 7.07 (d, J=7.6, 1H), 7.00-6.97 (m, 1H), 6.94 (d, J=8.9, 2H), 6.87 (dd, J=7.8, 2.1, 1H), 6.23 (s, 1H), 3.70 (s, 4H), 3.48 (s, 2H), 2.53-2.26 (m, 4H), 1.70-1.54 (m, 4H).

### Example 97

### 7-Benzyl-2,7-diaza-spiro[3.5]nonane-2-carboxylic acid pyridin-3-ylamide

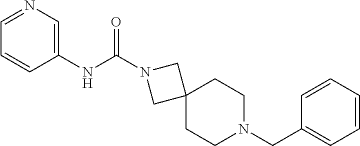

MS (ESI⁺): calcd for C₂₀H₂₄N₄O m/z 336.20; found 337.2 (M+H)⁺. ¹H NMR (CDCl₃): 8.42 (d, J=2.1, 1H), 8.25 (dd, J=4.7, 1.5, 1H), 8.07-8.03 (m, 1H), 7.33-7.28 (m, 5H), 7.24-7.19 (m, 1H), 3.76 (s, 4H), 3.47 (s, 2H), 2.36 (s, 4H), 1.81 (t, J=5.5, 4H).

### Example 98

### 7-(2-Chloro-benzyl)-2,7-diaza-spiro[3.5]nonane-2-carboxylic acid pyridin-3-ylamide

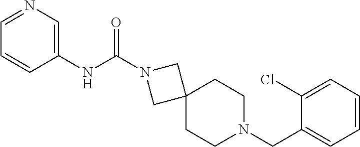

MS (ESI⁺): calcd for C₂₀H₂₃ClN₄O m/z 370.16; found 371.2 (M+H)⁺. ¹H NMR (CDCl₃): 8.43 (dd, J=2.7, 0.6, 1H), 8.26 (dd, J=4.7, 1.5, 1H), 8.08-8.04 (m, 1H), 7.45 (dd, J=7.5, 1.7, 1H), 7.35 (dd, J=7.7, 1.5, 1H), 7.24-7.16 (m, 3H), 3.78 (s, 4H), 3.59 (s, 2H), 2.44 (s, 4H), 1.83 (t, J=5.5, 4H).

### Example 99

### 7-(3-Chloro-benzyl)-2,7-diaza-spiro[3.5]nonane-2-carboxylic acid pyridin-3-ylamide

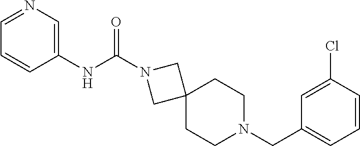

MS (ESI⁺): calcd for C₂₀H₂₃ClN₄O m/z 370.16; found 371.2 (M+H)⁺. ¹H NMR (CDCl₃): 8.43 (dd, J=2.7, 0.6, 1H), 8.25 (dd, J=4.7, 1.5, 1H), 8.08-8.03 (m, 1H), 7.32 (s, 1H), 7.24-7.15 (m, 4H), 3.76 (s, 4H), 3.44 (s, 2H), 2.36 (s, 4H), 1.81 (t, J=5.5, 4H).

### Example 100

### 7-(4-Chloro-benzyl)-2,7-diaza-spiro[3.5]nonane-2-carboxylic acid pyridin-3-ylamide

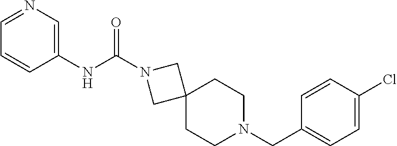

MS (ESI⁺): calcd for C₂₀H₂₃ClN₄O m/z 370.16; found 371.2 (M+H)⁺. ¹H NMR (CDCl₃): 8.42 (d, J=2.3, 1H), 8.26 (dd, J=4.7, 1.4, 1H), 8.07-8.03 (m, 1H), 7.30-7.26 (m, 2H), 7.25-7.20 (m, 3H), 6.04 (s, 1H), 3.76 (s, 4H), 3.49 (d, J=5.0, 2H), 2.35 (s, 4H), 1.80 (t, J=5.5, 4H).

### Example 101

### 7-(3,4-dichlorobenzyl)-N-(pyridin-3-yl)-2,7-diazaspiro[3.5]nonane-2-carboxamide

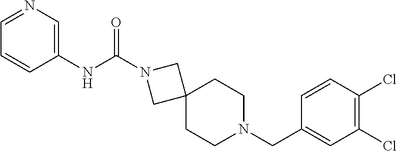

MS (ESI⁺): calcd for C₂₀H₂₂Cl₂N₄O m/z 404.12; found 405.2 (M+H)⁺. ¹H NMR (CDCl₃): 8.43 (dd, J=2.6, 0.5, 1H), 8.26 (dd, J=4.7, 1.5, 1H), 8.07-8.03 (m, 1H), 7.42 (d, J=1.9, 1H), 7.37 (d, J=8.2, 1H), 7.24-7.20 (m, 1H), 7.14 (dd, J=8.2, 2.0, 1H), 3.77 (s, 4H), 3.41 (s, 2H), 2.35 (s, 4H), 1.81 (t, J=5.5, 4H).

### Example 102

### N-(pyridin-3-yl)-7-(4-(trifluoromethyl)benzyl)-2,7-diazaspiro[3.5]nonane-2-carboxamide.

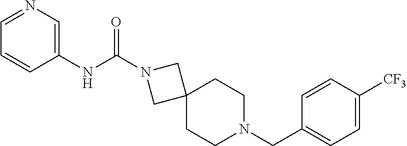

MS (ESI⁺): calcd for O₂₁H₂₃F₃N₄O m/z 404.18; found 405.2 (M+H)⁺. ¹H NMR (CDCl₃): 8.43 (dd, J=2.7, 0.5, 1H), 8.25 (dd, J=4.7, 1.5, 1H), 8.07-8.03 (m, 1H), 7.57 (d, J=8.0, 2H), 7.43 (d, J=8.0, 2H), 7.24-7.19 (m, 1H), 6.22 (s, 1H), 3.77 (s, 4H), 3.51 (s, 2H), 2.36 (s, 4H), 1.81 (t, J=5.4, 4H).

### Example 103

### N-(pyridin-3-yl)-7-(4-(trifluoromethoxy)benzyl)-2,7-diazaspiro[3.5]nonane-2-carboxamide.

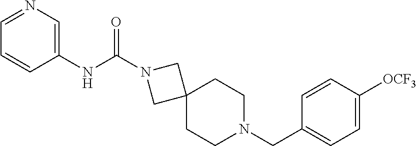

MS (ESI⁺): calcd for O₂₁H₂₃F₃N₄O₂ m/z 420.18; found 421.2 (M+H)⁺. ¹H NMR (CDCl₃): 8.43 (dd, J=2.7, 0.6, 1H), 8.25 (dd, J=4.7, 1.5, 1H), 8.07-8.04 (m, 1H), 7.33 (d, J=8.7, 2H), 7.24-7.20 (m, 1H), 7.18-7.14 (m, 2H), 3.77 (s, 4H), 3.46 (s, 2H), 2.35 (s, 4H), 1.81 (t, J=5.5, 4H).

### Example 104

### 7-(naphthalen-2-ylmethyl)-N-(pyridin-3-yl)-2,7-diazaspiro[3.5]nonane-2-carboxamide

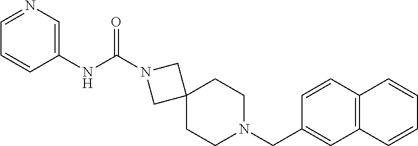

MS (ESI⁺): calcd for C₂₄H₂₆N₄O m/z 386.21; found 387.2 (M+H)⁺. ¹H NMR (CDCl₃): 8.43 (dd, J=2.7, 0.5, 1H), 8.25 (dd, J=4.7, 1.5, 1H), 8.07-8.03 (m, 1H), 7.84-7.78 (m, 3H), 7.71 (s, 1H), 7.49-7.43 (m, 3H), 7.23-7.19 (m, 1H), 3.76 (s, 4H), 3.63 (s, 2H), 2.41 (s, 4H), 1.82 (t, J=5.4, 4H).

### Example 105

### 7-(3-(phenylethynyl)benzyl)-N-(pyridin-3-yl)-2,7-diazaspiro[3.5]nonane-2-carboxamide.

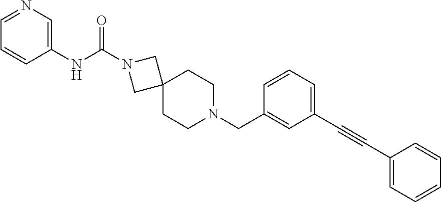

MS (ESI⁺): calcd for C₂₈H₂₈N₄O m/z 436.23; found 437.2 (M+H)⁺. ¹H NMR (CDCl₃): 8.46 (d, J=2.4, 1H), 8.26 (dd, J=4.7, 1.1, 1H), 8.08-8.04 (m, 1H), 7.56-7.45 (m, 4H), 7.38-7.29 (m, 5H), 7.24 (dd, J=8.4, 4.7, 1H), 6.25 (s, 1H), 3.78 (s, 4H), 3.63 (s, 2H), 2.52 (s, 4H), 1.91 (s, 4H).

### Example 106

### 7-(4-(methylsulfonyl)benzyl)-N-(pyridin-3-yl)-2,7-diazaspiro[3.5]nonane-2-carboxamide.

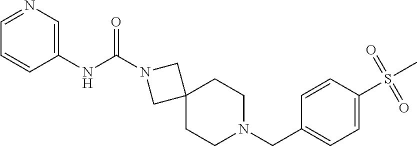

MS (ESI⁺): calcd for C₂₁H₂₆N₄O₃S m/z 414.17; found 415.2 (M+H)⁺. ¹H NMR (CDCl₃): 8.43 (d, J=2.6, 1H), 8.26 (dd, J=4.7, 1.4, 1H), 8.08-8.04 (m, 1H), 7.89 (d, J=8.3, 2H), 7.54 (d, J=8.3, 2H), 7.23 (dd, J=8.4, 4.7, 1H), 6.08 (s, 1H), 3.78 (s, 4H), 3.55 (s, 2H), 3.06 (s, 3H), 2.38 (s, 4H), 1.83 (t, J=5.4, 4H).

### Example 107

### 7-(2-hydroxybenzyl)-N-(pyridin-3-yl)-2,7-diazaspiro[3.5]nonane-2-carboxamide

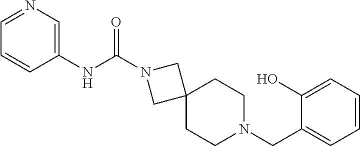

MS (ESI⁺): calcd for C₂₀H₂₄N₄O₂ m/z 352.19; found 353.2 (M+H)⁺. ¹H NMR (CDCl₃): 8.43 (d, J=2.6, 1H), 8.27 (dd, J=4.7, 1.4, 1H), 8.07-8.03 (m, 1H), 7.25-7.22 (m, 1H), 7.20-7.16 (m, 1H), 6.96 (d, J=6.1, 1H), 6.82 (dd, J=8.1, 1.0, 1H), 6.81-6.77 (m, 1H), 6.08 (s, 1H), 3.81 (s, 4H), 3.69 (s, 2H), 2.86-2.14 (br s, 4H), 1.93-1.85 (m, 4H).

### Example 108

### 7-((1-hydroxynaphthalen-2-yl)methyl)-N-(pyridin-3-yl)-2,7-diazaspiro[3.5]nonane-2-carboxamide.

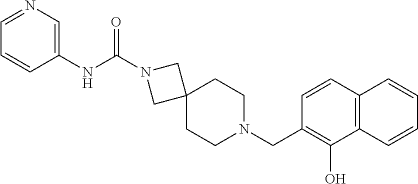

MS (ESI⁺): calcd for C₂₄H₂₆N₄O₂ m/z 402.21; found 403.3 (M+H)⁺. ¹H NMR (CDCl₃): 8.57 (d, J=2.1, 1H), 8.26-8.21 (m, 3H), 7.77-7.74 (m, 1H), 7.47-7.44 (m, 2H), 7.33-7.29 (m, 2H), 7.07 (d, J=8.3, 1H), 6.67 (s, 1H), 3.88 (s, 2H), 3.84 (s, 4H), 1.95 (t, J=5.2, 4H).

### Example 109

### 7-(4-chloro-3-(trifluoromethyl)benzyl)-N-(pyridin-3-yl)-2,7-diazaspiro[3.5]nonane-2-carboxamide.

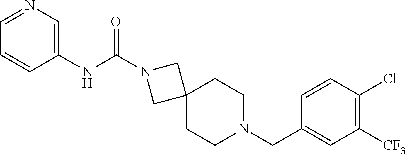

MS (ESI⁺): calcd for C₂₁H₂₂ClF₃N₄O m/z 438.15; found 439.2 (M+H)⁺. ¹H NMR (CDCl₃): 8.49 (d, J=2.4, 1H), 8.26 (dd, J=4.7, 1.1, 1H), 8.13-8.08 (m, 1H), 7.66 (s, 1H), 7.46 (s, 2H), 7.28-7.24 (m, 1H), 6.39 (s, 1H), 3.78 (s, 4H), 3.54 (s, 2H), 2.43 (s, 4H), 1.85 (t, J=5.3, 4H).

### Example 110

### 7-(4-chloro-3-(trifluoromethoxy)benzyl)-N-(pyridin-3-yl)-2,7-diazaspiro[3.5]nonane-2-carboxamide.

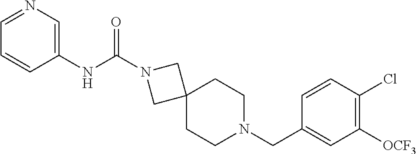

MS (ESI⁺): calcd for C₂₁H₂₂ClF₃N₄O₂ m/z 454.14; found 455.2 (M+H)⁺. ¹H NMR (CDCl₃): 8.55 (d, J=2.4, 1H), 8.24 (dd, J=4.8, 1.1, 1H), 8.19-8.14 (m, 1H), 7.43 (d, J=8.2, 1H), 7.36-7.34 (m, 1H), 7.28 (dd, J=8.5, 4.9, 1H), 7.23 (dd, J=8.2, 1.8, 1H), 6.65 (s, 1H), 3.79 (s, 4H), 3.61 (s, 2H), 2.52 (s, 4H), 1.89 (t, J=5.3, 4H).

### Example 111

### 7-(3-chloro-4-(trifluoromethoxy)benzyl)-N-(pyridin-3-yl)-2,7-diazaspiro[3.5]nonane-2-carboxamide.

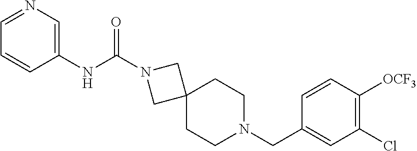

MS (ESI⁺): calcd for C₂₁H₂₂ClF₃N₄O₂ m/z 454.14; found 455.2 (M+H)⁺. ¹H NMR (CDCl₃): 8.51 (d, J=2.2, 1H), 8.25 (d, J=3.8, 1H), 8.15-8.11 (s, 1H), 7.47 (d, J=1.0, 1H), 7.27 (s, 2H), 6.47 (s, 1H), 3.79 (s, 4H), 3.55 (s, 2H), 2.48 (s, 4H), 1.88 (t, J=5.4, 4H).

### Example 112

### 7-(3-(3-chlorophenoxy)benzyl)-N-(pyridin-3-yl)-2,7-diazaspiro[3.5]nonane-2-carboxamide.

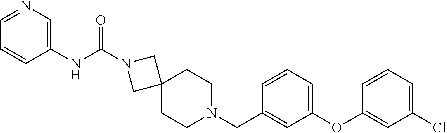

MS (ESI⁺): calcd for C₂₆H₂₇ClN₄O₂ m/z 462.18; found 463.2 (M+H)⁺. ¹H NMR (CDCl₃): 8.42 (d, J=2.5, 1H), 8.26 (dd, J=4.7, 1.4, 1H), 8.07-8.04 (m, 1H), 7.30 (t, J=7.9, 1H), 7.25-7.21 (m, 2H), 7.10-7.08 (m, 1H), 7.07-7.05 (m, 1H), 7.03-7.02 (m, 1H), 6.96 (t, J=2.1, 1H), 6.93-6.87 (m, 2H), 6.04 (s, 1H), 3.77 (s, 4H), 3.47 (s, 2H), 2.37 (s, 4H), 1.81 (t, J=5.4, 4H).

### Example 113

### 7-(3-(4-fluoro-3-(trifluoromethyl)phenoxy)benzyl)-N-(pyridin-3-yl)-2,7-diazaspiro[3.5]nonane-2-carboxamide.

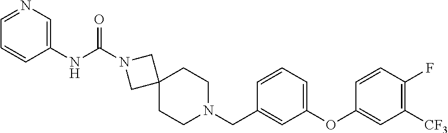

MS (ESI⁺): calcd for C₂₇H₂₆F₄N₄O₂ m/z 514.20; found 515.2 (M+H)⁺. ¹H NMR (CDCl₃): 8.42 (d, J=2.6, 1H), 8.27 (dd, J=4.7, 1.4, 1H), 8.07-8.04 (m, 1H), 7.31 (t, J=7.8, 1H), 7.25-7.19 (m, 2H), 7.17-7.15 (m, 2H), 7.09 (d, J=7.6, 1H), 7.01-6.99 (m, 1H), 6.88 (dd, J=8.1, 1.7, 1H), 5.97 (s, 1H), 3.77 (s, 4H), 3.47 (s, 2H), 2.37 (s, 4H), 1.81 (t, J=5.3, 4H).

### Example 114

### N-(pyridin-3-yl)-7-(3-(3-(trifluoromethyl)phenoxy)benzyl)-2,7-diazaspiro[3.5]nonane-2-carboxamide.

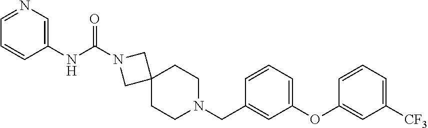

MS (ESI⁺): calcd for C₂₇H₂₇F₃N₄O₂ m/z 496.21; found 497.2 (M+H)⁺. ¹H NMR (CDCl₃): 8.42 (d, J=2.6, 1H), 8.26 (dd, J=4.7, 1.5, 1H), 8.07-8.04 (m, 1H), 7.44 (t, J=8.0, 1H), 7.35-7.29 (m, 2H), 7.24-7.20 (m, 2H), 7.17 (dd, J=8.2, 2.4, 1H), 7.11 (d, J=7.6, 1H), 7.04-7.03 (m, 1H), 6.92 (dd, J=8.1, 1.7, 1H), 6.01 (s, 1H), 3.76 (s, 4H), 3.47 (s, 2H), 2.37 (s, 4H), 1.81 (t, J=5.4, 4H).

### Example 115

### N-(pyridin-3-yl)-7-(3-(3-(trifluoromethoxy)phenoxy)benzyl)-2,7-diazaspiro[3.5]nonane-2-carboxamide.

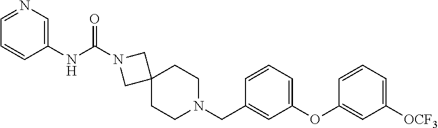

MS (ESI⁺): calcd for C₂₇H₂₇F₃N₄O₃ m/z 512.20; found 513.2 (M+H)⁺. ¹H NMR (CDCl₃): 8.45 (d, J=1.6, 1H), 8.24 (d, J=4.1, 1H), 8.07-8.03 (m, 1H), 7.32 (q, J=7.9, 7.6, 2H), 7.21 (dd, J=8.3, 4.7, 1H), 7.10 (d, J=7.6, 1H), 7.05-7.02 (m, 1H), 6.96-6.90 (m, 3H), 6.84-6.82 (m, 1H), 6.37 (s, 1H), 3.76 (s, 4H), 3.46 (s, 2H), 2.35 (s, 4H), 1.79 (t, J=5.4, 4H).

### Example 116

### 7-(3-(3-cyanophenoxy)benzyl)-N-(pyridin-3-yl)-2,7-diazaspiro[3.5]nonane-2-carboxamide.

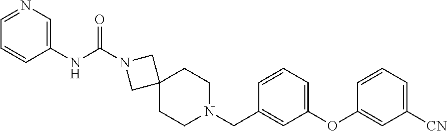

MS (ESI⁺): calcd for C₂₇H₂₇N₅O₂ m/z 453.22; found 454.2 (M+H)⁺. ¹H NMR (CDCl₃): 8.49 (d, J=2.5, 1H), 8.22 (d, J=4.7, 1H), 8.07-8.03 (m, 1H), 7.43 (t, J=8.0, 1H), 7.37-7.32 (m, 2H), 7.27-7.24 (m, 1H), 7.20 (dd, J=8.4, 4.7, 1H), 7.15-7.12 (m, 2H), 7.02 (s, 1H), 6.94-6.91 (m, 1H), 6.72 (s, 1H), 3.75 (s, 4H), 3.50 (s, 2H), 2.35 (s, 4H), 1.79 (t, J=5.3, 4H).

### Example 117

### N-(pyridin-3-yl)-7-(3-(3-(trifluoromethylthio)phenoxy)benzyl)-2,7-diazaspiro[3.5]nonane-2-carboxamide.

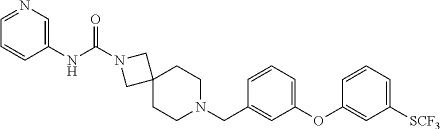

MS (ESI⁺): calcd for C₂₇H₂₇F₃N₄O₂S m/z 528.18; found 529.2 (M+H)⁺. ¹H NMR (CDCl₃): 8.45 (d, J=2.5, 1H), 8.23 (d, J=4.7, 1H), 8.08-8.04 (m, 1H), 7.59 (d, J=8.7, 2H), 7.32 (t, J=7.8, 1H), 7.22 (dd, J=8.4, 4.7, 1H), 7.12 (d, J=7.6, 1H), 7.07-7.04 (m, 1H), 6.98 (d, J=8.8, 2H), 6.96-6.93 (m, 1H), 6.65 (s, 1H), 3.75 (s, 4H), 3.48 (s, 2H), 2.37 (s, 4H), 1.79 (t, J=5.3, 4H).

### Example 118

### 7-[3-(2,2-Difluoro-benzo[1,3]-dioxol-5-yloxy)-benzyl]-2,7-diaza-spiro[3.5]nonane-2-carboxylic acid pyridin-3-ylamide.

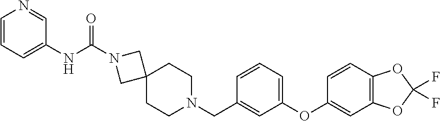

MS (ESI⁺): calcd for C₂₇H₂₆F₂N₄O₄ m/z 508.19; found 509.2 (M+H)⁺. ¹H NMR (CDCl₃): 8.45 (d, J=2.5, 1H), 8.23 (dd, J=4.7, 1.2, 1H), 8.09-8.05 (m, 1H), 7.28 (t, J=7.9, 1H), 7.22 (dd, J=8.4, 4.8, 1H), 7.06 (d, J=7.6, 1H), 7.01-6.97 (m, 2H), 6.86 (dd, J=7.9, 2.2, 1H), 6.77 (d, J=2.4, 1H), 6.71 (dd, J=8.7, 2.4, 1H), 6.65 (s, 1H), 3.75 (s, 4H), 3.47 (s, 2H), 2.37 (s, 4H), 1.79 (t, J=5.4, 4H).

### Example 119

### 7-(3-Phenoxy-benzyl)-2,7-diaza-spiro[3.5]nonane-2-carboxylic acid pyridin-3-ylamide

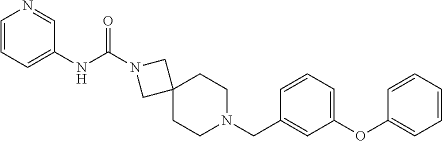

MS (ESI⁺): calcd for C₂₆H₂₈N₄O₂ m/z 428.22; found 429.3 (M+H)⁺. ¹H NMR (CDCl₃): 8.45 (d, J=2.5, 1H), 8.21 (dd, J=4.7, 1.2, 1H), 8.07-8.04 (m, 1H), 7.35-7.31 (m, 2H), 7.26 (dd, J=8.8, 6.9, 1H), 7.21 (dd, J=8.4, 4.7, 1H), 7.10 (t, J=7.4, 1H), 7.04 (d, J=7.6, 1H), 7.02-6.98 (m, 3H), 6.89 (dd, J=8.0, 2.3, 1H), 6.78 (s, 1H), 3.73 (s, 4H), 3.46 (s, 2H), 2.35 (s, 4H), 1.77 (t, J=5.4, 4H).

### Example 120

### 7-[3-(4-Cyano-3-trifluoromethyl-phenoxy)-benzyl]-2,7-diaza-spiro[3.5]nonane-2-carboxylic acid pyridin-3-ylamide.

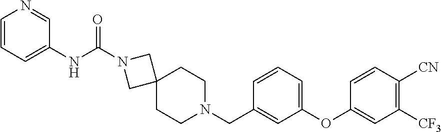

MS (ESI⁺): calcd for C₂₈H₂₆F₃N₅O₂ m/z 521.20; found 522.2 (M+H)⁺. ¹H NMR (CDCl₃): 8.43 (d, J=2.6, 1H), 8.25 (dd, J=4.7, 1.4, 1H), 8.06-8.02 (m, 1H), 7.75 (d, J=8.6, 1H), 7.39 (t, J=7.8, 1H), 7.31 (d, J=2.4, 1H), 7.24-7.19 (m, 2H), 7.14 (dd, J=8.6, 2.5, 1H), 7.10-7.07 (m, 1H), 6.97 (dd, J=8.3, 2.1, 1H), 6.14 (s, 1H), 3.76 (s, 4H), 3.48 (s, 2H), 2.37 (s, 4H), 1.81 (t, J=5.4, 4H).

### Example 121

### 7-[3-(2-Chloro-phenoxy)-benzyl]-2,7-diaza-spiro[3.5]nonane-2-carboxylic acid pyridin-3-ylamide

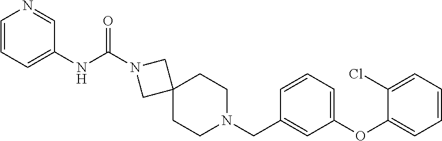

MS (ESI⁺): calcd for C₂₆H₂₇ClN₄O₂ m/z 462.18; found 563.2 (M+H)⁺. ¹H NMR (CDCl₃): 8.42 (d, J=2.6, 1H), 8.27-8.22 (m, 1H), 8.07-8.01 (m, 1H), 7.48-7.42 (m, 1H), 7.24-7.18 (m, 2H), 7.11-7.02 (m, 2H), 6.99-6.94 (m, 2H), 6.85-6.80 (m, 1H), 6.20 (s, 1H), 3.75 (s, 4H), 3.45 (s, 2H), 2.35 (s, 4H), 1.79 (t, J=5.1, 4H).

### Example 122

### 7-[3-(3-Bromo-phenoxy)-benzyl]-2,7-diaza-spiro[3.5]nonane-2-carboxylic acid pyridin-3-ylamide

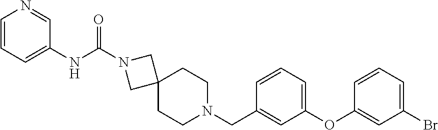

MS (ESI⁺): calcd for C₂₆H₂₇BrN₄O₂ m/z 506.13; found 507.2 (M+H)⁺. ¹H NMR (CDCl₃): 8.42 (d, J=2.6, 1H), 8.25 (dd, J=4.7, 1.4, 1H), 8.08-8.04 (m, 1H), 7.30 (t, J=7.8, 1H), 7.24-7.17 (m, 3H), 7.13-7.11 (m, 1H), 7.09 (d, J=7.6, 1H), 7.03-7.01 (m, 1H), 6.95-6.89 (m, 2H), 6.21 (s, 1H), 3.76 (s, 4H), 3.46 (s, 2H), 2.36 (s, 4H), 1.80 (t, J=5.4, 4H).

### Example 123

### 7-[3-(4-Bromo-phenoxy)-benzyl]-2,7-diaza-spiro[3.5]nonane-2-carboxylic acid pyridin-3-ylamide

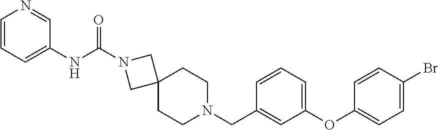

MS (ESI⁺): calcd for C₂₆H₂₇BrN₄O₂ m/z 506.13; found 507.2 (M+H)⁺. ¹H NMR (CDCl₃): 8.42 (d, J=2.6, 1H), 8.25 (dd, J=4.7, 1.4, 1H), 8.07-8.03 (m, 1H), 7.42 (d, J=8.8, 2H), 7.30-7.26 (m, 1H), 7.22 (dd, J=8.4, 4.7, 1H), 7.07 (d, J=7.6, 1H), 7.00-6.98 (m, 1H), 6.88 (d, J=8.8, 3H), 6.22 (s, 1H), 3.76 (s, 4H), 3.45 (s, 2H), 2.35 (s, 4H), 1.80 (t, J=5.3, 4H).

### Example 124

### 7-[3-(3,4-Difluoro-phenoxy)-benzyl]-2,7-diaza-spiro[3.5]nonane-2-carboxylic acid pyridin-3-ylamide

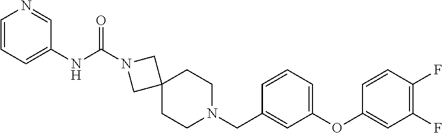

MS (ESI⁺): calcd for C₂₆H₂₆F₂N₄O₂ m/z 464.20 found 465.2 (M+H)⁺. ¹H NMR (CDCl₃): 8.43 (d, J=2.2, 1H), 8.25 (dd, J=4.7, 1.5, 1H), 8.07-8.02 (m, 1H), 7.31-7.26 (m, 1H), 7.21 (dd, J=8.4, 4.7, 1H), 7.14-7.06 (m, 2H), 7.00-6.98 (m, 1H), 6.87 (dd, J=8.1, 1.7, 1H), 6.84-6.78 (m, 1H), 6.75-6.69 (m, 1H), 6.24 (s, 1H), 3.75 (s, 4H), 3.45 (s, 2H), 2.35 (s, 4H), 1.79 (t, J=5.4, 4H).

### Example 125

### 7-[3-(4-Trifluoromethoxy-phenoxy)-benzyl]-2,7-diaza-spiro[3.5]nonane-2-carboxylic acid pyridin-3-ylamide.

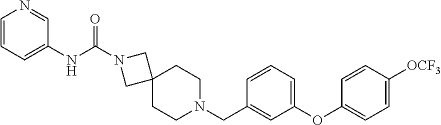

MS (ESI⁺): calcd for C₂₇H₂₇F₃N₄O₃ m/z 512.20; found 513.2 (M+H)⁺. ¹H NMR (CDCl₃): 8.42 (d, J=2.3, 1H), 8.25 (dd, J=4.7, 1.4, 1H), 8.07-8.03 (m, 1H), 7.28 (t, J=7.9, 1H), 7.21 (dd, J=8.4, 4.7, 1H), 7.19-7.16 (m, 2H), 7.08 (d, J=7.7, 1H), 7.02-6.97 (m, 3H), 6.89 (dd, J=8.1, 1.6, 1H), 6.13 (s, 1H), 3.75 (s, 4H), 3.46 (s, 2H), 2.36 (s, 4H), 1.80 (t, J=5.4, 4H).

### Example 126

### 7-[3-(4-Trifluoromethyl-phenoxy)-benzyl]-2,7-diaza-spiro[3.5]nonane-2-carboxylic acid pyridin-3-ylamide

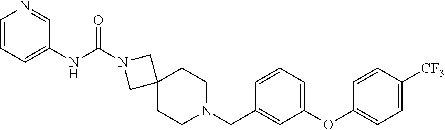

MS (ESI⁺): calcd for C₂₇H₂₇F₃N₄O₂ m/z 496.21; found 497.2 (M+H)⁺. ¹H NMR (CDCl₃): 8.44 (d, J=2.6, 1H), 8.23 (dd, J=4.7, 1.4, 1H), 8.06-8.02 (m, 1H), 7.57 (d, J=8.6, 2H), 7.32 (t, J=7.8, 1H), 7.20 (dd, J=8.4, 4.7, 1H), 7.13 (d, J=7.6, 1H), 7.06-7.01 (m, 3H), 6.93 (dd, J=8.4, 2.0, 1H), 6.65 (s, 1H), 3.74 (s, 4H), 3.46 (s, 2H), 2.36 (s, 4H), 1.78 (t, J=5.4, 4H).

### Example 127

### 7-[3-(4-Cyano-phenoxy)-benzyl]-2,7-diaza-spiro[3.5]nonane-2-carboxylic acid pyridin-3-ylamide

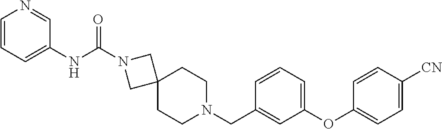

MS (ESI⁺): calcd for C₂₇H₂₇N₅O₂ m/z 453.22; found 454.3 (M+H)⁺. ¹H NMR (CDCl₃): 8.46 (d, J=2.6, 1H), 8.24 (dd, J=4.7, 1.4, 1H), 8.06-8.03 (m, 1H), 7.60 (d, J=8.9, 2H), 7.35 (t, J=7.8, 1H), 7.21 (dd, J=8.4, 4.7, 1H), 7.16 (d, J=7.6, 1H), 7.07-7.05 (m, 1H), 7.00 (d, J=8.8, 2H), 6.95 (dd, J=7.9, 2.1, 1H), 6.59 (s, 1H), 3.76 (s, 4H), 3.47 (s, 2H), 2.35 (s, 4H), 1.79 (t, J=5.4, 4H).

### Example 128

### 7-{3-[4-(2,2,2-Trifluoro-ethoxy)-phenoxy]-benzyl}-2,7-diaza-spiro[3.5]nonane-2-carboxylic acid pyridin-3-ylamide.

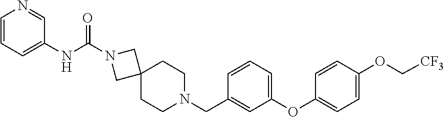

MS (ESI⁺): calcd for C₂₈H₂₉F₃N₄O₃ m/z 526.22; found 527.3 (M+H)⁺. ¹H NMR (CDCl₃): 8.44 (d, J=2.6, 1H), 8.24 (dd, J=4.7, 1.4, 1H), 8.06-8.02 (m, 1H), 7.24 (t, J=7.9, 1H), 7.21 (dd, J=8.4, 4.8, 1H), 7.03-6.91 (m, 6H), 6.82 (dd, J=8.1, 2.4, 1H), 6.50 (s, 1H), 4.34 (q, J=8.2, 8.2, 2H), 3.74 (s, 4H), 3.43 (s, 2H), 2.34 (s, 4H), 1.78 (t, J=5.4, 4H).

### Example 129

### 7-[3-(4-Trifluoromethanesulfonyl-phenoxy)-benzyl]-2,7-diaza-spiro[3.5]nonane-2-carboxylic acid pyridin-3-ylamide.

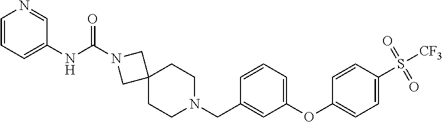

MS (ESI⁺): calcd for C₂₇H₂₇F₃N₄O₄S m/z 560.17; found 561.2 (M+H)⁺. ¹H NMR (CDCl₃): 8.45 (d, J=2.6, 1H), 8.24 (dd, J=4.7, 1.4, 1H), 8.05-8.01 (m, 1H), 7.95 (d, J=8.9, 2H), 7.39 (t, J=7.9, 1H), 7.24-7.19 (m, 2H), 7.14-7.10 (m, 3H), 7.00 (dd, J=8.1, 1.7, 1H), 6.56 (s, 1H), 3.76 (s, 4H), 3.49 (s, 2H), 2.36 (s, 4H), 1.79 (t, J=5.3, 4H).

### Example 130

### 7-[3-(Quinolin-6-yloxy)-benzyl]-2,7-diaza-spiro[3.5]nonane-2-carboxylic acid pyridin-3-ylamide

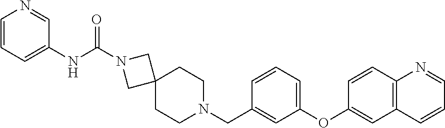

MS (ESI⁺): calcd for C₂₉H₂₉N₅O₂ m/z 479.23; found 480.3 (M+H)⁺. ¹H NMR (CDCl₃): 8.83 (dd, J=4.2, 1.6, 1H), 8.44 (d, J=2.6, 1H), 8.23 (dd, J=4.7, 1.4, 1H), 8.09 (d, J=9.2, 1H), 8.06-8.03 (m, 1H), 8.01 (d, J=7.6, 1H), 7.48 (dd, J=9.1, 2.7, 1H), 7.37 (dd, J=8.3, 4.2, 1H), 7.33 (t, J=7.8, 7.8, 1H), 7.23 (d, J=2.7, 1H), 7.21 (dd, J=8.4, 4.7, 1H), 7.12 (d, J=7.6, 1H), 7.09-7.08 (m, 1H), 6.98 (dd, J=8.0, 2.3, 1H), 6.58 (s, 1H), 3.74 (s, 4H), 3.47 (s, 2H), 2.35 (s, 4H), 1.77 (t, J=5.3, 4H).

### Example 131

### 7-[3-(2-Chloro-phenylethynyl)-benzyl]-2,7-diaza-spiro[3.5]nonane-2-carboxylic acid pyridin-3-ylamide

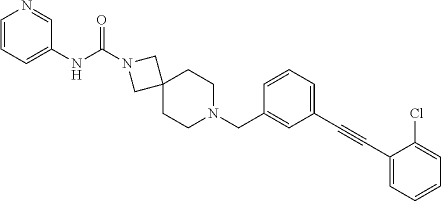

MS (ESI⁺): calcd for C₂₈H₂₇ClN₄O m/z 470.19; found 471.2 (M+H)⁺. ¹H NMR (CDCl₃): 8.42 (d, J=2.6, 1H), 8.26 (dd, J=4.7, 1.4, 1H), 8.08-8.04 (m, 1H), 7.56 (dd, J=7.2, 2.2, 1H), 7.52 (s, 1H), 7.49-7.46 (m, 1H), 7.45-7.42 (m, 1H), 7.33-7.29 (m, 2H), 7.25-7.20 (m, 2H), 6.09 (s, 1H), 3.77 (s, 4H), 3.48 (s, 2H), 2.38 (s, 4H), 1.82 (t, J=5.4, 4H).

### Biological Testing:

Assay Method 1

A. Transfection of Cells with Human FAAH

A 10-cm tissue culture dish with a confluent monolayer of SK-N-MC cells was split 2 days (d) prior to transfection. Using sterile technique, the media was removed and the cells were detached from the dish by the addition of trypsin. One fifth of the cells were then placed onto a new 10-cm dish. Cells were grown in a 37° C. incubator with 5% CO₂ in Minimal Essential Media Eagle with 10% Fetal Bovine Serum. After 2 d, cells were approximately 80% confluent. These cells were removed from the dish with trypsin and pelleted in a clinical centrifuge. The pellet was re-suspended in 400 μL complete media and transferred to an electroporation cuvette with a 0.4 cm gap between the electrodes. Supercoiled human FAAH cDNA (1 μg) was added to the cells and mixed. The voltage for the electroporation was set at 0.25 kV, and the capacitance was set at 960 μF. After electroporation, the cells were diluted into complete media (10 mL) and plated onto four 10-cm dishes. Because of the variability in the efficiency of electroporation, four different concentrations of cells were plated. The ratios used were 1:20, 1:10, and 1:5, with the remainder of the cells being added to the fourth dish. The cells were allowed to recover for 24 h before adding the selection media (complete media with 600 μg/mL G418). After 10 d, dishes were analyzed for surviving colonies of cells. Dishes with well-isolated colonies were used. Cells from individual colonies were isolated and tested. The clones that showed the most FAAH activity, as measured by anandamide hydrolysis, were used for further study.

B. FAAH Assay

T84 frozen cell pellets or transfected SK-N-MC cells (contents of 1×15 cm culture dishes) were homogenized in 50 mL of FAAH assay buffer (125 mM Tris, 1 mM EDTA, 0.2% Glycerol, 0.02% Triton X-100, 0.4 mM Hepes, pH 9). The assay mixture consisted of 50 μL of the cell homogenate, 10 μL of the test compound, and 40 μL of anandamide [1-³H-ethanolamine] (³H-AEA, Perkin-Elmer, 10.3 Cᵢ/mmol), which was added last, for a final tracer concentration of 80 nM. The reaction mixture was incubated at rt for 1 h. During the incubation, 96-well Multiscreen filter plates (catalog number MAFCNOB50; Millipore, Bedford, Mass., USA) were loaded with 25 of activated charcoal (Multiscreen column loader, catalog number MACL09625, Millipore) and washed once with 100 μL of MeOH. Also during the incubation, 96-well DYNEX MicroLite plates (catalog number NL510410) were loaded with 100 μL of MicroScint40 (catalog number 6013641, Packard Bioscience, Meriden, Conn., USA). After the 1 h incubation, 60 μL of the reaction mixture were transferred to the charcoal plates, which were then assembled on top of the DYNEX plates using Centrifuge Alignment Frames (catalog number MACF09604, Millipore). The unbound labeled ethanolamine was centrifuged through to the bottom plate (5 min at 2000 rpm), which was preloaded with the scintillant, as described above. The plates were sealed and left at rt for 1 h before counting on a Hewlett Packard TopCount.

Assay Method 2

A. Transfection of Cells with Rat FAAH

A 10-cm tissue culture dish with a confluent monolayer of SK-N-MC cells was split 2 days (d) prior to transfection. Using sterile technique, the media was removed and the cells were detached from the dish by the addition of trypsin. One fifth of the cells were then placed onto a new 10-cm dish. Cells were grown in a 37° C. incubator with 5% CO₂ in Minimal Essential Media Eagle with 10% Fetal Bovine Serum. After 2 d, cells were approximately 80% confluent. These cells were removed from the dish with trypsin and pelleted in a clinical centrifuge. The pellet was re-suspended in 400 μL complete media and transferred to an electroporation cuvette with a 0.4 cm gap between the electrodes. Supercoiled rat FAAH cDNA (1 μg) was added to the cells and mixed. The voltage for the electroporation was set at 0.25 kV, and the capacitance was set at 960 μF. After electroporation, the cells were diluted into complete media (10 mL) and plated onto four 10-cm dishes. Because of the variability in the efficiency of electroporation, four different concentrations of cells were plated. The ratios used were 1:20, 1:10, and 1:5, with the remainder of the cells being added to the fourth dish. The cells were allowed to recover for 24 h before adding the selection media (complete media with 600 μg/mL G418). After 10 d, dishes were analyzed for surviving colonies of cells. Dishes with well-isolated colonies were used. Cells from individual colonies were isolated and tested. The clones that showed the most FAAH activity, as measured by anandamide hydrolysis, were used for further study.

B. FAAH Assay

T84 frozen cell pellets or transfected SK-N-MC cells (contents of 1×15 cm culture dishes) were homogenized in 50 mL of FAAH assay buffer (125 mM Tris, 1 mM EDTA, 0.2% Glycerol, 0.02% Triton X-100, 0.4 mM Hepes, pH 9). The assay mixture consisted of 50 μL of the cell homogenate, 10 μL of the test compound, and 40 μL of anandamide [1-³H-ethanolamine] (³H-AEA, Perkin-Elmer, 10.3 Cᵢ/mmol), which was added last, for a final tracer concentration of 80 nM. The reaction mixture was incubated at rt for 1 h. During the incubation, 96-well Multiscreen filter plates (catalog number MAFCNOB50; Millipore, Bedford, Mass., USA) were loaded with 25 of activated charcoal (Multiscreen column loader, catalog number MACL09625, Millipore) and washed once with 100 μL of MeOH. Also during the incubation, 96-well DYNEX MicroLite plates (catalog number NL510410) were loaded with 100 μL of MicroScint40 (catalog number 6013641, Packard Bioscience, Meriden, Conn., USA). After the 1 h incubation, 60 μL of the reaction mixture were transferred to the charcoal plates, which were then assembled on top of the DYNEX plates using Centrifuge Alignment Frames (catalog number MACF09604, Millipore). The unbound labeled ethanolamine was centrifuged through to the bottom plate (5 min at 2000 rpm), which was preloaded with the scintillant, as described above. The plates were sealed and left at rt for 1 h before counting on a Hewlett Packard TopCount.

Results for compounds tested in these assays are summarized in Table 1, as an average of results obtained. Compounds were tested in free base or trifluoroacetic acid salt forms. Where activity is shown as greater than (>) a particular value, the value is the solubility limit of the compound in the assay medium or the highest concentration tested in the assay.

TABLE 1

| | Assay 1 | Assay 2 |
|---|---|---|
| Ex. | IC₅₀ (μM) | IC₅₀ (μM) |
| 1 | 0.02 | 0.06 |
| 2 | 0.13 | 0.01 |
| 3 | 0.05 | 0.34 |
| 4 | 0.03 | 0.23 |
| 5 | 0.13 | 0.01 |
| 6 | 0.01 | 0.02 |
| 7 | 9.00 | 10.00 |
| 8 | 8.00 | 3.00 |
| 9 | 1.00 | 0.32 |
| 10 | 1.50 | 0.13 |
| 11 | 0.17 | 0.03 |
| 12 | 0.70 | 0.10 |
| 13 | 0.03 | 0.20 |
| 14 | 0.03 | 0.05 |
| 15 | 0.04 | 0.06 |
| 16 | >10 | 6.00 |
| 17 | 3.00 | 0.43 |
| 18 | >10 | 5.00 |
| 19 | 1.00 | 0.70 |
| 20 | >10 | >10 |
| 21 | 4.00 | 5.00 |
| 22 | 6.00 | 1.10 |
| 23 | 1.00 | 0.50 |
| 24 | >10 | 0.30 |
| 25 | 2.30 | 0.58 |
| 26 | 10.00 | 6.00 |
| 27 | 0.80 | 0.20 |
| 28 | 0.32 | 0.01 |
| 29 | 0.22 | 0.01 |
| 30 | 1.60 | 0.12 |
| 31 | 0.01 | 0.00 |
| 32 | 10.00 | 0.01 |
| 33 | 0.07 | 0.01 |
| 34 | >10 | 0.12 |
| 35 | 0.03 | 0.70 |
| 36 | 0.01 | 0.03 |
| 37 | 0.09 | 0.06 |
| 38 | 0.82 | 0.02 |
| 39 | 0.02 | 0.02 |
| 40 | 0.06 | 0.04 |
| 41 | 0.02 | 0.08 |
| 42 | >10 | 1.00 |
| 43 | 5.66 | 5.44 |
| 44 | 0.43 | 0.26 |
| 45 | 0.04 | 0.10 |
| 46 | 1.00 | 1.00 |
| 47 | 10.00 | >10 |
| 48 | 1.50 | 8.00 |
| 49 | 1.80 | 8.00 |
| 50 | 0.82 | 8.00 |
| 51 | 10.00 | 0.20 |
| 52 | 0.66 | 0.67 |
| 53 | 0.01 | 0.01 |
| 54 | 0.17 | 0.19 |
| 55 | 6.93 | 6.93 |
| 56 | 3.69 | 1.67 |
| 57 | 0.26 | 0.20 |
| 58 | 0.30 | 0.69 |
| 59 | 0.25 | 0.08 |
| 60 | 0.30 | 0.12 |
| 61 | >10 | >10 |
| 62 | >10 | >10 |
| 63 | 1.45 | 0.50 |
| 64 | 3.16 | 0.06 |
| 65 | 0.24 | 1.06 |
| 66 | 0.13 | 0.60 |
| 67 | 0.16 | 0.01 |
| 68 | >10 | >10 |
| 69 | >10 | >10 |
| 70 | 3.00 | 2.40 |
| 71 | >10 | 5.00 |
| 72 | 1.70 | 0.06 |
| 73 | 0.08 | 0.45 |
| 74 | >10 | 2.00 |
| 75 | 2.50 | 0.40 |
| 76 | >10 | >10 |
| 77 | >10 | >10 |
| 78 | >10 | >10 |
| 79 | 0.07 | 0.03 |
| 80 | 0.14 | 0.57 |
| 81 | 1.60 | 3.00 |
| 82 | 0.28 | 0.02 |
| 83 | 0.01 | 0.02 |
| 84 | 0.11 | 0.01 |
| 85 | 0.08 | 0.04 |
| 86 | 0.42 | 0.04 |
| 87 | 10.00 | >10 |
| 88 | 4.00 | >10 |
| 89 | 0.50 | 10.00 |
| 90 | 2.00 | 0.43 |
| 91 | 0.20 | 0.26 |
| 92 | 0.16 | 0.02 |
| 93 | 1.10 | 0.48 |
| 94 | 0.37 | 0.58 |
| 95 | 0.78 | 0.40 |
| 96 | 8.00 | 6.00 |
| 97 | >10 | >10 |
| 98 | 10.00 | >10 |
| 99 | 10.00 | >10 |
| 100 | 8.00 | >10 |
| 101 | 6.00 | >10 |
| 102 | 10.00 | >10 |
| 103 | 1.30 | >10 |
| 104 | 2.50 | >10 |
| 105 | 0.37 | 8.00 |
| 106 | >10 | >10 |
| 107 | >10 | >10 |
| 108 | 8.00 | >10 |
| 109 | 10.00 | >10 |
| 110 | 1.30 | 6.00 |
| 111 | 1.18 | >10 |
| 112 | 0.06 | 0.84 |
| 113 | 0.56 | 5.00 |
| 114 | 0.80 | 4.00 |
| 115 | 0.75 | 8.00 |
| 116 | 0.07 | 7.07 |
| 117 | 3.00 | 3.00 |
| 118 | 0.16 | 0.60 |
| 119 | 0.10 | 4.00 |
| 120 | 10.00 | 10.00 |
| 121 | 0.03 | 1.20 |
| 122 | 0.05 | 1.00 |
| 123 | 0.20 | 0.03 |
| 124 | 0.30 | 0.40 |
| 125 | 2.60 | 3.00 |
| 126 | 0.16 | 1.00 |
| 127 | 4.00 | 1.10 |
| 128 | 8.00 | 10.00 |
| 129 | 1.70 | 8.00 |
| 130 | 0.83 | 2.50 |
| 131 | 0.33 | 3.00 |

While the invention has been illustrated by reference to exemplary and preferred embodiments, it will be understood that the invention is intended not to be limited to the foregoing detailed description, but to be defined by the appended claims as properly construed under principles of patent law.

What is claimed is:

1. A composition of matter selected from compounds of Formula (I), pharmaceutically acceptable salts of compounds of Formula (I), and pharmaceutically acceptable prodrugs of compounds of Formula (I), 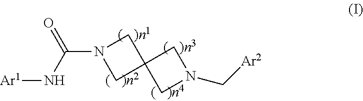
wherein
n¹, n², n³, and n⁴, in the form of sets [n¹,n²,n³,n⁴], are chosen from the following sets, [2,2,1,2], [2,2,1,1], [2,1,0,3], [1,2,1,2], [2,2,2,2], [1,3,2,1], [1,2,2,2], [1,3,2,2], [2,2,1,3], [1,3,3,1], [1,3,1,1], [1,1,2,2], [1,1,1,1], [2,2,0,3], or [1,1,1,3];
Ar¹ is benzo[1,2,5]oxadiazolyl, benzo[d]isoxazolyl, benzooxazol-yl, benzo[d]thiazolyl, imidazo[1,2-a]pyridinyl, imidazo[1,2-b]pyridazinyl, 1H-indazolyl, isoxazolyl, isoxazolo[4,5-b]pyridinyl, isoxazolo[5,4-b]pyridinyl, phenyl, pyrazolyl, 1H-pyrazolo[3,4-b]pyridinyl, pyridazinyl, pyridyl, pyrimidinyl, 1H-pyrrolo[2,3-b]pyridinyl, quinolinyl, or tetrazolyl, with the point of attachment being any substitutable carbon of the respective heterocycle;
where each Ar¹ is optionally substituted with one or two groups, each said group individually selected from —C₁₋₃alkyl, halo, —CF₃, —CN, —OC₁₋₃alkyl, triazolyl, phenyl, morpholinyl, piperidinyl, or pyrazolyl;
Ar² is
(i) phenyl optionally substituted with one or two R^{a} moieties;
where each R^{a} moiety is independently —OH, —CN, halo, —CF₃, —O(CH₂)₀₋₁CF₃, —S(O)(O)C₁₋₄alkyl, —SCF₃, —S(O)(O)CF₃, or two adjacent R^{a} moieties taken together form —OCF₂O—;
(ii) phenyl substituted at the 3-position with -L-Ar³,
where L is a linker selected from the group consisting of —O— or —C≡C—; and
Ar³ is:
(c) phenyl optionally substituted with one or two R^{a} moieties; or
(d) quinolinyl; or
(iii) napthyl optionally substituted with —OH.

2. A composition of matter as in claim 1, wherein Ar¹ is 6-[1,2,3]triazol-2-yl-pyridin-3-yl, benzo[d]isoxazol-3-yl, pyridin-3yl, 1H-pyrrolo[2,3-b]pyridin-5-yl, 4-chloropyridin-3-yl, 5-fluoro-pyridin-3yl, quinolin-3-yl, 4-[1,2,3]triazol-2-yl-phenyl, 6-pyrazol-1-yl-pyridin-3-yl, 5-methylpyridin-3-yl, 4-methylpyridin-3-yl, or 2-phenylpyrimidin-5-yl.

3. A composition of matter as in claim 2, wherein n¹, n², n³, and n⁴ are chosen from the following sets [1,1,1,1], [1,1,2,2], [2,2,1,1], [2,2,1,2], or [2,2,2,2].

4. A composition of matter as in claim 1, wherein Ar² is phenyl substituted at the 3-position with -L-Ar³.

5. A composition of matter as in claim 4, wherein L is —O—.

6. A composition of matter as in claim 4, wherein Ar³ is phenyl optionally substituted with one or two R^{a} moieties.

7. A composition of matter as in claim 6, wherein said R^{a} moieties are selected from the group consisting of F, Cl, Br, —CF₃, —OCF₃, —CN, —SO₂CF₃, —SCF₃, and —OCH₂CF₃.

8. A composition of matter as in claim 4, wherein L is —O— and Ar³ is phenyl optionally substituted with one or two R^{a} moieties.

9. A composition of matter as in claim 8, wherein Ar¹ is 6-[1,2,3]triazol-2-yl-pyridin-3-yl, benzo[d]isoxazol-3-yl, pyridin-3yl, 1H-pyrrolo[2,3-b]pyridin-5-yl, 4-chloropyridin-3-yl, 5-fluoro-pyridin-3yl, quinolin-3-yl, 4-[1,2,3]triazol-2-yl-phenyl, 6-pyrazol-1-yl-pyridin-3-yl, 5-methylpyridin-3-yl, 4-methylpyridin-3-yl, or 2-phenylpyrimidin-5-yl.

10. A composition of matter as in claim 9, wherein n¹, n², n³, and n⁴ are chosen from the following sets [1,1,1,1], [1,1,2,2], [2,2,1,1], [2,2,1,2], or [2,2,2,2].

11. A composition of matter as in claim 4, wherein L is —C≡O—.

12. A composition of matter as in claim 11, wherein Ar¹ is 6-[1,2,3]triazol-2-yl-pyridin-3-yl, benzo[d]isoxazol-3-yl, pyridin-3yl, 1H-pyrrolo[2,3-b]pyridin-5-yl, 4-chloropyridin-3-yl, 5-fluoro-pyridin-3yl, quinolin-3-yl, 4-[1,2,3]triazol-2-yl-phenyl, 6-pyrazol-1-yl-pyridin-3-yl, 5-methylpyridin-3-yl, 4-methylpyridin-3-yl, or 2-phenylpyrimidin-5-yl.

13. A composition of matter as in claim 1, selected from the group consisting of:
2-[3-(4-Chloro-phenoxy)-benzyl]-2,8-diaza-spiro[4.5]decane-8-carboxylic acid (6-[1,2,3]triazol-2-yl-pyridin-3-yl)-amide;
2-[3-(4-Chloro-phenoxy)-benzyl]-2,8-diaza-spiro[4.5]decane-8 carboxylic acid benzo[d]isoxazol-3-ylamide;
2-[3-(4-Chloro-phenoxy)-benzyl]-2,8-diaza-spiro[4.5]decane-8-carboxylic acid pyridin-3-ylamide;
2-[3-(4-Chloro-phenoxy)-benzyl]-2,7-diaza-spiro[3.5]nonane-7-carboxylic acid pyridin-3-ylamide;
2-[3-(4-Chloro-phenoxy)-benzyl]-2,7-diaza-spiro[3.5]nonane-7-carboxylic acid benzo[d]isoxazol-3-ylamide;
2-[3-(4-Chloro-phenoxy)-benzyl]-2,7-diaza-spiro[3.5]nonane-7-carboxylic acid (6-[1,2,3]triazol-2-yl-pyridin-3-yl)-amide;
1-[3-(4-Chloro-phenoxy)-benzyl]-1,7-diaza-spiro[4.4]nonane-7-carboxylic acid pyridin-3-ylamide;
1-[3-(4-Chloro-phenoxy)-benzyl]-1,7-diaza-spiro[4.4]nonane-7-carboxylic acid (6-[1,2,3]triazol-2-yl-pyridin-3-yl)-amide;
1-[3-(4-Chloro-phenoxy)-benzyl]-1,7-diaza-spiro[4.4]nonane-7-carboxylic acid (1H-pyrrolo[2,3-b]pyridin-5-yl)-amide;
7-[3-(4-Chloro-phenoxy)-benzyl]-2,7-diaza-spiro[4.4]nonane-2-carboxylic acid pyridin-3-ylamide;
7-[3-(4-Chloro-phenoxy)-benzyl]-2,7-diaza-spiro[4.4]nonane-2-carboxylic acid (6-[1,2,3]triazol-2-yl-pyridin-3-yl)-amide;
7-[3-(4-Chloro-phenoxy)-benzyl]-2,7-diaza-spiro[4.4]nonane-2-carboxylic acid (1H-pyrrolo[2,3-b]pyridin-5-yl)-amide;
9-[3-(4-Chloro-phenoxy)-benzyl]-3,9-diaza-spiro[5.5]undecane-3-carboxylic acid pyridin-3-ylamide;
9-[3-(4-Chloro-phenoxy)-benzyl]-3,9-diaza-spiro[5.5]undecane-3-carboxylic acid (6-[1,2,3]triazol-2-yl-pyridin-3-yl)-amide;
9-[3-(4-Chloro-phenoxy)-benzyl]-3,9-diaza-spiro[5.5]undecane-3-carboxylic acid (1H-pyrrolo[2,3-b]pyridin-5-yl)-amide;
2-(3-(4-chlorophenoxy)benzyl)-N-(pyridin-3-yl)-2,7-diazaspiro[4.5]decane-7-carboxamide;
N-(6-(2H-1,2,3-triazol-2-yl)pyridin-3-yl)-2-(3-(4-chlorophenoxy)benzyl)-2,7-diazaspiro[4.5]decane-7-carboxamide;
8-(3-(4-chlorophenoxy)benzyl)-N-(pyridin-3-yl)-2,8-diazaspiro[4.5]decane-2-carboxamide;
N-(6-(2H-1,2,3-triazol-2-yl)pyridin-3-yl)-8-(3-(4-chlorophenoxy)benzyl)-2,8-diazaspiro[4.5]decane-2-carboxamide;
9-(3-(4-chlorophenoxy)benzyl)-N-(pyridin-3-yl)-2,9-diazaspiro[5.5]undecane-2-carboxamide;
N-(6-(2H-1,2,3-triazol-2-yl)pyridin-3-yl)-9-(3-(4-chlorophenoxy)benzyl)-2,9-diazaspiro[5.5]undecane-2-carboxamide;
2-(3-(4-chlorophenoxy)benzyl)-N-(pyridin-3-yl)-2,9-diazaspiro[5.5]undecane-9-carboxamide;
N-(6-(2H-1,2,3-triazol-2-yl)pyridin-3-yl)-2-(3-(4-chlorophenoxy)benzyl)-2,9-diazaspiro[5.5]undecane-9-carboxamide;
8-(3-(4-chlorophenoxy)benzyl)-N-(pyridin-3-yl)-2,8-diazaspiro[5.5]undecane-2-carboxamide;
N-(6-(2H-1,2,3-triazol-2-yl)pyridin-3-yl)-8-(3-(4-chlorophenoxy)benzyl)-2,8-diazaspiro[5.5]undecane-2-carboxamide;
2-(3-(4-chlorophenoxy)benzyl)-N-(pyridin-3-yl)-2,6-diazaspiro[3.5]nonane-6-carboxamide;
N-(6-(2H-1,2,3-triazol-2-yl)pyridin-3-yl)-2-(3-(4-chlorophenoxy)benzyl)-2,6-diazaspiro[3.5]nonane-6-carboxamide;
7-(3-(4-chlorophenoxy)benzyl)-N-(imidazo[1,2-b]pyridazin-3-yl)-2,7-diazaspiro[3.5]nonane-2-carboxamide;
7-(3-(4-chlorophenoxy)benzyl)-N-(imidazo[1,2-a]pyridin-3-yl)-2,7-diazaspiro[3.5]nonane-2-carboxamide;
2-(3-(4-chlorophenoxy)benzyl)-N-(4-chloropyridin-3-yl)-2,6-diazaspiro[3.5]nonane-6-carboxamide;
9-(3-(4-chlorophenoxy)benzyl)-N-(4-chloropyridin-3-yl)-3,9-diazaspiro[5.5]undecane-3-carboxamide;
2-(3-(4-chlorophenoxy)benzyl)-N-(quinolin-3-yl)-2,6-diazaspiro[3.5]nonane-6-carboxamide;
9-(3-(4-chlorophenoxy)benzyl)-N-(quinolin-3-yl)-3,9-diazaspiro[5.5]undecane-3-carboxamide;
2-(3-(4-chlorophenoxy)benzyl)-N-(imidazo[1,2-a]pyridin-3-yl)-2,6-diazaspiro[3.5]nonane-6-carboxamide;
6-(3-(4-chlorophenoxy)benzyl)-N-(pyridin-3-yl)-2,6-diazaspiro[3.3]heptane-2-carboxamide;
6-(3-(4-chlorophenoxy)benzyl)-N-(4-chloropyridin-3-yl)-2,6-diazaspiro[3.3]heptane-2-carboxamide;
7-[3-(4-Chloro-phenoxy)-benzyl]-2,7-diaza-spiro[3.5]nonane-2-carboxylic acid pyridin-3-ylamide;
7-[3-(4-Chloro-phenoxy)-benzyl]-2,7-diaza-spiro[3.5]nonane-2-carboxylic acid benzo[d]isoxazol-3-ylamide;
7-[3-(4-Chloro-phenoxy)-benzyl]-2,7-diaza-spiro[3.5]nonane-2-carboxylic acid (6-[1,2,3]triazol-2-yl-pyridin-3-yl)-amide;
7-[3-(4-Chloro-phenoxy)-benzyl]-2,7-diaza-spiro[3.5]nonane-2-carboxylic acid (1H-pyrrolo[2, 3-1D]pyridin-5-yl)-amide;
7-[3-(4-Chloro-phenoxy)-benzyl]-2,7-diaza-spiro[3.5]nonane-2-carboxylic acid (4-[1,2,3]triazol-2-yl-phenyl)-amide;
7-[3-(4-Chloro-phenoxy)-benzyl]-2,7-diaza-spiro[3.5]nonane-2-carboxylic acid pyrimidin-2-ylamide;
7-[3-(4-Chloro-phenoxy)-benzyl]-2,7-diaza-spiro[3.5]nonane-2-carboxylic acid pyrimidin-4-ylamide;
7-[3-(4-Chloro-phenoxy)-benzyl]-2,7-diaza-spiro[3.5]nonane-2-carboxylic acid pyridazin-3-ylamide;
7-[3-(4-Chloro-phenoxy)-benzyl]-2,7-diaza-spiro[3.5]nonane-2-carboxylic acid (6-pyrazol-1-yl-pyridin-3-yl)-amide;
7-[3-(4-Chloro-phenoxy)-benzyl]-2,7-diaza-spiro[3.5]nonane-2-carboxylic acid (6-[1,2,4]triazol-1-yl-pyridin-3-yl)-amide;
7-[3-(4-Chloro-phenoxy)-benzyl]-2,7-diaza-spiro[3.5]nonane-2-carboxylic acid (6-[1,2,4]triazol-4-yl-pyridin-3-yl)-amide;
7-[3-(4-Chloro-phenoxy)-benzyl]-2,7-diaza-spiro[3.5]nonane-2-carboxylic acid (6-chloro-pyridin-3-yl)-amide;
7-[3-(4-Chloro-phenoxy)-benzyl]-2,7-diaza-spiro[3.5]nonane-2-carboxylic acid (6-methoxy-pyridin-3-yl)-amide;
7-[3-(4-Chloro-phenoxy)-benzyl]-2,7-diaza-spiro[3.5]nonane-2-carboxylic acid (6-cyano-pyridin-3-yl)-amide;
7-[3-(4-Chloro-phenoxy)-benzyl]-2,7-diaza-spiro[3.5]nonane-2-carboxylic acid (1H-tetrazol-5-yl)-amide;
7-[3-(4-Chloro-phenoxy)-benzyl]-2,7-diaza-spiro[3.5]nonane-2-carboxylic acid benzo[1,2,5]oxadiazol-4-ylamide;
7-[3-(4-Chloro-phenoxy)-benzyl]-2,7-diaza-spiro[3.5]nonane-2-carboxylic acid (4-chloro-pyridin-3-yl)-amide;
7-[3-(4-Chloro-phenoxy)-benzyl]-2,7-diaza-spiro[3.5]nonane-2-carboxylic acid (2-chloro-pyridin-3-yl)-amide;
7-[3-(4-Chloro-phenoxy)-benzyl]-2,7-diaza-spiro[3.5]nonane-2-carboxylic acid (6-morpholin-4-yl-pyridin-3-yl)-amide;
7-[3-(4-Chloro-phenoxy)-benzyl]-2,7-diaza-spiro[3.5]nonane-2-carboxylic acid (1H-pyrazol-3-yl)-amide;
7-[3-(4-Chloro-phenoxy)-benzyl]-2,7-diaza-spiro[3.5]nonane-2-carboxylic acid (5-chloro-pyridin-3-yl)-amide;
7-[3-(4-Chloro-phenoxy)-benzyl]-2,7-diaza-spiro[3.5]nonane-2-carboxylic acid (6-fluoro-pyridin-3-yl)-amide;
7-[3-(4-Chloro-phenoxy)-benzyl]-2,7-diaza-spiro[3.5]nonane-2-carboxylic acid (6-methoxy-pyrimidin-4-yl)-amide;
7-[3-(4-Chloro-phenoxy)-benzyl]-2,7-diaza-spiro[3.5]nonane-2-carboxylic acid (6-chloro-pyridazin-3-yl)-amide;
7-[3-(4-Chloro-phenoxy)-benzyl]-2,7-diaza-spiro[3.5]nonane-2-carboxylic acid (1,5-dimethyl-1H-pyrazol-3-yl)-amide;
7-[3-(4-Chloro-phenoxy)-benzyl]-2,7-diaza-spiro[3.5]nonane-2-carboxylic acid (4-bromo-1-methyl-1H-pyrazol-3-yl)-amide;
7-[3-(4-Chloro-phenoxy)-benzyl]-2,7-diaza-spiro[3.5]nonane-2-carboxylic acid (2-ethyl-2H-pyrazol-3-yl)-amide;
7-[3-(4-Chloro-phenoxy)-benzyl]-2,7-diaza-spiro[3.5]nonane-2-carboxylic acid (2H-tetrazol-5-yl)-amide;
7-[3-(4-Chloro-phenoxy)-benzyl]-2,7-diaza-spiro[3.5]nonane-2-carboxylic acid (2-methyl-benzooxazol-5-yl)-amide;
7-[3-(4-Chloro-phenoxy)-benzyl]-2,7-diaza-spiro[3.5]nonane-2-carboxylic acid isoxazolo[5,4-b]pyridin-3-ylamide;
7-[3-(4-Chloro-phenoxy)-benzyl]-2,7-diaza-spiro[3.5]nonane-2-carboxylic acid isoxazolo[4,5-b]pyridin-3-ylamide;
7-[3-(4-Chloro-phenoxy)-benzyl]-2,7-diaza-spiro[3.5]nonane-2-carboxylic acid (1H-indazol-7-yl)-amide;
7-[3-(4-Chloro-phenoxy)-benzyl]-2,7-diaza-spiro[3.5]nonane-2-carboxylic acid imidazo[1,2-a]pyridin-6-ylamide;
7-[3-(4-Chloro-phenoxy)-benzyl]-2,7-diaza-spiro[3.5]nonane-2-carboxylic acid (6-methoxy-pyridazin-3-yl)-amide;
7-[3-(4-Chloro-phenoxy)-benzyl]-2,7-diaza-spiro[3.5]nonane-2-carboxylic acid (2-trifluoromethyl-pyrimidin-4-yl)-amide;
7-[3-(4-Chloro-phenoxy)-benzyl]-2,7-diaza-spiro[3.5]nonane-2-carboxylic acid (2-methoxy-pyrimidin-4-yl)-amide;
7-[3-(4-Chloro-phenoxy)-benzyl]-2,7-diaza-spiro[3.5]nonane-2-carboxylic acid (5-fluoro-pyridin-3-yl)-amide;
7-[3-(4-Chloro-phenoxy)-benzyl]-2,7-diaza-spiro[3.5]nonane-2-carboxylic acid (1H-pyrrolo[2,3-b]pyridin-4-yl)-amide;
7-(3-(4-chlorophenoxy)benzyl)-N-(1,3-dimethyl-1H-pyrazolo[3,4-b]pyridin-5-yl)-2,7-diazaspiro[3.5]nonane-2-carboxamide;
7-(3-(4-chlorophenoxy)benzyl)-N-(5-methylisoxazol-3-yl)-2,7-diazaspiro[3.5]nonane-2-carboxamide;
7-(3-(4-chlorophenoxy)benzyl)-N-(2-methylbenzo[d]thiazol-6-yl)-2,7-diazaspiro[3.5]nonane-2-carboxamide;
7-(3-(4-chlorophenoxy)benzyl)-N-(5-methyl-1H-pyrazol-3-yl)-2,7-diazaspiro[3.5]nonane-2-carboxamide;
7-(3-(4-chlorophenoxy)benzyl)-N-(5-methylpyridin-3-yl)-2,7-diazaspiro[3.5]nonane-2-carboxamide;
7-(3-(4-chlorophenoxy)benzyl)-N-(2-fluoropyridin-3-yl)-2,7-diazaspiro[3.5]nonane-2-carboxamide;
7-(3-(4-chlorophenoxy)benzyl)-N-(6-(piperidin-1-yl)pyridin-3-yl)-2,7-diazaspiro[3.5]nonane-2-carboxamide;
N-(5-bromopyridin-3-yl)-7-(3-(4-chlorophenoxy)benzyl)-2,7-diazaspiro[3.5]nonane-2-carboxamide;
7-(3-(4-chlorophenoxy)benzyl)-N-(2-phenylpyrimidin-5-yl)-2,7-diazaspiro[3.5]nonane-2-carboxamide;
7-(3-(4-chlorophenoxy)benzyl)-N-(4-cyanopyridin-3-yl)-2,7-diazaspiro[3.5]nonane-2-carboxamide;
7-(3-(4-chlorophenoxy)benzyl)-N-(4-methylpyridin-3-yl)-2,7-diazaspiro[3.5]nonane-2-carboxamide;
7-(3-(4-chlorophenoxy)benzyl)-N-(4-(trifluoromethyl)pyridin-3-yl)-2,7-diazaspiro[3.5]nonane-2-carboxamide;
7-(2,2-Difluoro-benzo[1,3]dioxol-5-ylmethyl)-2,7-diaza-spiro[3.5]nonane-2-carboxylic acid pyridin-3-ylamide;
7-(2,2-Difluoro-benzo[1,3]dioxol-5-ylmethyl)-2,7-diaza-spiro[3.5]nonane-2-carboxylic acid (6-[1,2,3]triazol-2-yl-pyridin-3-yl)-amide;
7-(2,2-Difluoro-benzo[1,3]dioxol-5-ylmethyl)-2,7-diaza-spiro[3.5]nonane-2-carboxylic acid (1H-pyrrolo[2,3-b]pyridin-5-yl)-amide;
8-[3-(4-Chloro-phenoxy)-benzyl]-2,8-diaza-spiro[4.5]decane-2-carboxylic acid pyridin-3-ylamide;
8-[3-(4-Chloro-phenoxy)-benzyl]-2,8-diaza-spiro[4.5]decane-2-carboxylic acid (6-[1,2,3]triazol-2-yl-pyridin-3-yl)-amide;
8-[3-(4-Chloro-phenoxy)-benzyl]-2,8-diaza-spiro[4.5]decane-2-carboxylic acid benzo[d]isoxazol-3-ylamide;
1-[3-(4-Chloro-phenoxy)-benzyl]-1,8-diaza-spiro[4.5]decane-8-carboxylic acid pyridin-3-ylamide;
1-[3-(4-Chloro-phenoxy)-benzyl]-1,8-diaza-spiro[4.5]decane-8-carboxylic acid (6-[1,2,3]triazol-2-yl-pyridin-3-yl)-amide;
1-[3-(4-Chloro-phenoxy)-benzyl]-1,8-diaza-spiro[4.5]decane-8-carboxylic acid (1H-pyrrolo[2,3-b]pyridin-5-yl)-amide;
6-[3-(4-Chloro-phenoxy)-benzyl]-2,6-diaza-spiro[3.5]nonane-2-carboxylic acid pyridin-3-ylamide;
7-Benzyl-2,7-diaza-spiro[3.5]nonane-2-carboxylic acid pyridin-3-ylamide;
7-(2-Chloro-benzyl)-2,7-diaza-spiro[3.5]nonane-2-carboxylic acid pyridin-3-ylamide;
7-(3-Chloro-benzyl)-2,7-diaza-spiro[3.5]nonane-2-carboxylic acid pyridin-3-ylamide;
7-(4-Chloro-benzyl)-2,7-diaza-spiro[3.5]nonane-2-carboxylic acid pyridin-3-ylamide;
7-(3,4-dichlorobenzyl)-N-(pyridin-3-yl)-2,7-diazaspiro[3.5]nonane-2-carboxamide;
N-(pyridin-3-yl)-7-(4-(trifluoromethyl)benzyl)-2,7-diazaspiro[3.5]nonane-2-carboxamide;
N-(pyridin-3-yl)-7-(4-(trifluoromethoxy)benzyl)-2,7-diazaspiro[3.5]nonane-2-carboxamide;
7-(naphthalen-2-ylmethyl)-N-(pyridin-3-yl)-2,7-diazaspiro[3.5]nonane-2-carboxamide;
7-(3-(phenylethynyl)benzyl)-N-(pyridin-3-yl)-2,7-diazaspiro[3.5]nonane-2-carboxamide;
7-(4-(methylsulfonyl)benzyl)-N-(pyridin-3-yl)-2,7-diazaspiro[3.5]nonane-2-carboxamide;
7-(2-hydroxybenzyl)-N-(pyridin-3-yl)-2,7-diazaspiro[3.5]nonane-2-carboxamide;
7-((1-hydroxynaphthalen-2-yl)methyl)-N-(pyridin-3-yl)-2,7-diazaspiro[3.5]nonane-2-carboxamide;
7-(4-chloro-3-(trifluoromethyl)benzyl)-N-(pyridin-3-yl)-2,7-diazaspiro[3.5]nonane-2-carboxamide;
7-(4-chloro-3-(trifluoromethoxy)benzyl)-N-(pyridin-3-yl)-2,7-diazaspiro[3.5]nonane-2-carboxamide;
7-(3-chloro-4-(trifluoromethoxy)benzyl)-N-(pyridin-3-yl)-2,7-diazaspiro[3.5]nonane-2-carboxamide;
7-(3-(3-chlorophenoxy)benzyl)-N-(pyridin-3-yl)-2,7-diazaspiro[3.5]nonane-2-carboxamide;
7-(3-(4-fluoro-3-(trifluoromethyl)phenoxy)benzyl)-N-(pyridin-3-yl)-2,7-diazaspiro[3.5]nonane-2-carboxamide;
N-(pyridin-3-yl)-7-(3-(3-(trifluoromethyl)phenoxy)benzyl)-2,7-diazaspiro[3.5]nonane-2-carboxamide;
N-(pyridin-3-yl)-7-(3-(3-(trifluoromethoxy)phenoxy)benzyl)-2,7-diazaspiro[3.5]nonane-2-carboxamide;
7-(3-(3-cyanophenoxy)benzyl)-N-(pyridin-3-yl)-2,7-diazaspiro[3.5]nonane-2-carboxamide;
N-(pyridin-3-yl)-7-(3-(3-(trifluoromethylthio)phenoxy)benzyl)-2,7-diazaspiro[3.5]nonane-2-carboxamide;
7-[3-(2,2-Difluoro-benzo[1,3]dioxol-5-yloxy)-benzyl]-2,7-diaza-spiro[3.5]nonane-2-carboxylic acid pyridin-3-ylamide;
7-(3-Phenoxy-benzyl)-2,7-diaza-spiro[3.5]nonane-2-carboxylic acid pyridin-3-ylamide;
7-[3-(4-Cyano-3-trifluoromethyl-phenoxy)-benzyl]-2,7-diaza-spiro[3.5]nonane-2-carboxylic acid pyridin-3-ylamide;
7-[3-(2-Chloro-phenoxy)-benzyl]-2,7-diaza-spiro[3.5]nonane-2-carboxylic acid pyridin-3-ylamide;
7-[3-(3-Bromo-phenoxy)-benzyl]-2,7-diaza-spiro[3.5]nonane-2-carboxylic acid pyridin-3-ylamide;
7-[3-(4-Bromo-phenoxy)-benzyl]-2,7-diaza-spiro[3.5]nonane-2-carboxylic acid pyridin-3-ylamide;
7-[3-(3,4-Difluoro-phenoxy)-benzyl]-2,7-diaza-spiro[3.5]nonane-2-carboxylic acid pyridin-3-ylamide;
7-[3-(4-Trifluoromethoxy-phenoxy)-benzyl]-2,7-diaza-spiro[3.5]nonane-2-carboxylic acid pyridin-3-ylamide;
7-[3-(4-Trifluoromethyl-phenoxy)-benzyl]-2,7-diaza-spiro[3.5]nonane-2-carboxylic acid pyridin-3-ylamide;
7-[3-(4-Cyano-phenoxy)-benzyl]-2,7-diaza-spiro[3.5]nonane-2-carboxylic acid pyridin-3-ylamide;
7-{3-[4-(2,2,2-Trifluoro-ethoxy)-phenoxy]-benzyl}-2,7-diaza-spiro[3.5]nonane-2-carboxylic acid pyridin-3-ylamide;
7-[3-(4-Trifluoromethanesulfonyl-phenoxy)-benzyl]-2,7-diaza-spiro[3.5]nonane-2-carboxylic acid pyridin-3-ylamide;
7-[3-(Quinolin-6-yloxy)-benzyl]-2,7-diaza-spiro[3.5]nonane-2-carboxylic acid pyridin-3-ylamide;
7-[3-(2-Chloro-phenylethynyl)-benzyl]-2,7-diaza-spiro[3.5]nonane-2-carboxylic acid pyridin-3-ylamide;
and pharmaceutically acceptable slats and prodrugs thereof.

14. A pharmaceutical composition comprising:
(a) a therapeutically effective amount of a chemical entity selected from the group consisting of compounds of Formula (I): 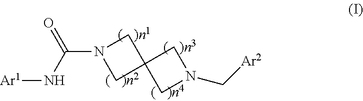
wherein
n¹, n², n³, and n⁴, in the form of sets [n¹,n²,n³,n⁴], are chosen from the following sets, [2,2,1,2], [2,2,1,1], [2,1,0,3], [1,2,1,2], [2,2,2,2], [1,3,2,1], [1,2,2,2], [1,3,2,2], [2,2,1,3], [1,3,3,1], [1,3,1,1], [1,1,2,2], [1,1,1,1], [2,2,0,3], or [1,1,1,3];
Ar¹ is benzo[1,2,5]oxadiazolyl, benzo[d]isoxazolyl, benzooxazol-yl, benzo[d]thiazolyl, imidazo[1,2-a]pyridinyl, imidazo[1,2-b]pyridazinyl, 1H-indazolyl, isoxazolyl, isoxazolo[4,5-b]pyridinyl, isoxazolo[5,4-b]pyridinyl, phenyl, pyrazolyl, 1H-pyrazolo[3,4-b]pyridinyl, pyridazinyl, pyridyl, pyrimidinyl, 1H-pyrrolo[2,3-b]pyridinyl, quinolinyl, or tetrazolyl, with the point of attachment being any substitutable carbon of the respective heterocycle;
where each Ar¹ is optionally substituted with one or two groups, each said group individually selected from —C₁₋₃alkyl, halo, —CF₃, —CN, —OC₁₋₃alkyl, triazolyl, phenyl, morpholinyl, piperidinyl, or pyrazolyl;
Ar² is
(i) phenyl optionally substituted with one or two R^{a} moieties;
where each R^{a} moiety is independently —OH, —CN, halo, —CF₃, —O(CH₂)₀₋₁CF₃, —S(O)(O)C₁₋₄alkyl, —SCF₃, —S(O)(O)CF₃, or two adjacent R^{a} moieties taken together form —OCF₂O—;
(ii) phenyl substituted at the 3-position with -L-Ar³,
where L is a linker selected from the group consisting of —O— or —C≡C—; and
Ar³ is:
(a) phenyl optionally substituted with one or two R^{a} moieties; or
(b) quinolinyl; or
(iii) napthyl optionally substituted with —OH;
and pharmaceutically acceptable salts of compounds of Formula (I),
pharmaceutically acceptable prodrugs of compounds of Formula (I); and
(b) a pharmaceutically acceptable excipient.

15. A pharmaceutical composition according to claim 14, further comprising: an analgesic selected from the group consisting of opioids and non-steroidal anti-inflammatory drugs.

16. A pharmaceutical composition according to claim 14, further comprising: an additional active ingredient selected from the group consisting of aspirin, acetaminophen, opioids, ibuprofen, naproxen, COX-2 inhibitors, gabapentin, pregabalin, and tramadol.

17. A method for modulating FAAH activity, comprising exposing FAAH to an effective amount of at least one chemical entity as defined in claim 14.

18. A method for treating a subject suffering from or diagnosed with a disease, disorder, or medical condition mediated by FAAH activity, comprising administering to the subject in need of such treatment an effective amount of at least one chemical entity as defined in claim 14.

19. A method according to claim 18, wherein the disease, disorder, or medical condition is selected from the group consisting of: anxiety, depression, pain, sleep disorders, eating disorders, inflammation, movement disorders, HIV wasting syndrome, closed head injury, stroke, learning and memory disorders, Alzheimer's disease, epilepsy, Tourette's syndrome, Niemann-Pick disease, Parkinson's disease, Huntington's chorea, optic neuritis, autoimmune uveitis, drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, autoimmune diabetes, intractable pruritis, neuroinflammation, diabetes, metabolic syndrome, and osteoporosis.

20. A method according to claim 18, wherein the disease, disorder, or medical condition is pain or inflammation.

21. A method according to claim 18, wherein the disease, disorder, or medical condition is anxiety, a sleep disorder, an eating disorder, or a movement disorder.

22. A method according to claim 18, wherein the disease, disorder, or medical condition is multiple sclerosis.

23. A method according to claim 18, wherein the disease, disorder, or medical condition is energy metabolism or bone homeostasis.
